(19) United States
(12) Patent Application Publication
(10) Pub. No.: US 2012/0122843 A1
(43) Pub. Date: May 17, 2012

(54) Compounds and Their Use for Treatment of Amyloid Beta-Related Diseases

(75) Inventors: István MACSÁRI, Sodertalje (SE); Kim PAULSEN, Sodertalje (SE); Laszlo RAKOS, Sodertalje (SE); Britt-Marie SWAHN, Sodertalje (SE); Magnus WALDMAN, Sodertalje (SE); Didier ROTTICCI, Sodertalje (SE); Stefan VON BERG, Sodertalje (SE)

(73) Assignee: ASTRAZENECA AB, Sodertalje (SE)

(21) Appl. No.: 13/292,582

(22) Filed: Nov. 9, 2011

Related U.S. Application Data
(60) Provisional application No. 61/412,472, filed on Nov. 11, 2010.

(51) Int. Cl. A61K 31/553; A61P 25/18; A61P 25/28; C07D 498/04; A61P 29/00

(52) U.S. Cl. 514/211.1; 540/552

(57) ABSTRACT

The present invention relates to novel compounds of formula (I) and pharmaceutically acceptable salts thereof, pharmaceutical compositions comprising said compounds, processes for making said compounds, and their use as medicaments for treatment and/or prevention of Aβ-related diseases.

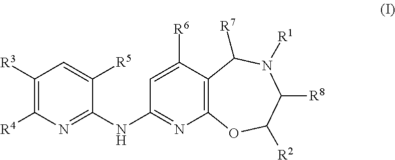

The present invention relates to pyrido[3,2-f][1,4]oxazepine compounds and pharmaceutically acceptable salts thereof. The present invention also relates to pharmaceutical compositions comprising said compounds, processes for making said compounds and their use as medicaments for treatment and/or prevention of various Aβ-related diseases.

### BACKGROUND

The prime neuropathological event distinguishing Alzheimer's disease (AD) is deposition of the amyloid β-peptide (Aβ) in brain parenchyma and cerebral vessels. A large body of genetic, biochemical and in vivo data support a pivotal role for Aβ in the pathological cascade that eventually leads to AD. Patients usually present early symptoms (commonly memory loss) in their sixth or seventh decades of life. The disease progresses with increasing dementia and elevated deposition of Aβ. In parallel, a hyperphosphorylated form of the microtubule-associated protein tau accumulates within neurons, leading to a plethora of deleterious effects on neuronal function. The prevailing working hypothesis regarding the temporal relationship between Aβ and tau pathologies states that Aβ deposition precedes tau aggregation in humans and animal models of the disease. Within this context, it is worth noting that the exact molecular nature of Aβ, mediating this pathological function is presently an issue under intense study. Most likely, there is a continuum of toxic species ranging from lower order Aβ oligomers to supramolecular assemblies such as Aβ fibrils.

The Aβ peptide is an integral fragment of the Type I protein APP (Aβ amyloid precursor protein), a protein ubiquitously expressed in human tissues. Aβ can be found in both plasma, cerebrospinal fluid (CSF), and in the medium from cultured cells, and is generated as a result of APP proteolysis. There are two main cleavages of APP that results in Aβ production, the so-called β-, and γ-cleavages. The β-cleavage, which generates the N terminus of Aβ, is catalyzed by the transmembrane aspartyl protease BACE1. The γ-cleavage, generating the Aβ C termini and subsequent release of the peptide, is affected by a multi-subunit aspartyl protease named γ-secretase. Both BACE1 and γ-secretase process APP at different sites, resulting in Aβ peptides of different lengths and heterologous N- and C-termini. The invention described herein covers all N-terminal variants of Aβ. Therefore, for the sake of simplicity, all N-terminal variants will be covered by the denotation Aβ.

The activity of γ-secretase causes the liberation of many Aβ peptides, such as Aβ37, Aβ38, Aβ39, Aβ40, Aβ42 and Aβ43, of which Aβ40 is the most common. These peptides show a different propensity to aggregate, and in particular Aβ42 is prone to form oligomers and fibrillar deposits. Intriguingly, human genetics strongly support a key role for Aβ42 as a key mediator of Alzheimer pathogenesis. Indeed, more than 150 different mutations causing familial Alzheimer's disease either result in an increase in the ratio of Aβ 42/40 peptides produced or affect the intrinsic aggregation behaviour of Aβ. Based on this knowledge, Aβ42 has become a prime target for therapeutic intervention in AD (Beher D, Curr Top Med Chem 2008; 8(1):34-7). Targeting Aβ42 at the level of γ-secretase activity must, however, be conducted with caution since γ-secretase catalyses proteolysis of many proteins, which have important physiological functions. Among its many substrates is the Notch receptor family, which signaling is essential for many different cell fate determination processes e.g. during embryogenesis and in the adult. As such, Aβ42 lowering strategies at the level of γ-secretase must be compatible with maintained Notch signaling.

It has been suggested that it is possible to combine γ-secretase interference and lowered Aβ42 production without obtaining toxic side effects due to impaired Notch signaling. There have, for instance, been reports which postulate that allosteric modulation of γ-secretase combines lowered Aβ42 production with maintained Notch signaling (Weggen et al. Nature 414(6860), 212-216 (2003); Kounnas et al. Neuron 67, 769-780 (2010); Zettl et al. Trends Pharmacol. Sci. 31, 402-410 (2010)). In addition, a number of compounds interfering with γ-secretase and Aβ production have been suggested in, e.g., WO2005/054193, WO2005/013985, WO2004/073705, WO2007/135969, WO2007/139149, WO2005/115990, WO2008/097538, WO2008/099210, WO2008/100412, WO2007/125364, WO2009/020580, WO2010/053438 and WO2010/132015.

The present invention relates to novel compounds which inhibit the Aβ40 and Aβ42 production, increase Aβ37 and Aβ38 levels and maintain Notch signaling. These compounds are therefore useful in the prevention and/or treatment of, e.g., Alzheimer's Disease (AD).

### DISCLOSURE OF THE INVENTION

In one aspect, the invention relates to a compound of formula (I)

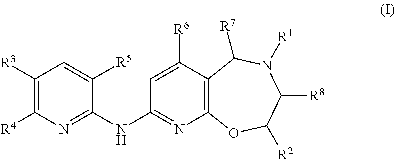

wherein:
R¹ is selected from hydrogen, C₁₋₃-alkyl, —C(O)CH₃, —CH₂CH₂OCH₃, —C(O)N(CH₃)₂ and —CH₂CN;
R² is selected from C₂₋₄-alkyl (optionally substituted with one or more substituents independently selected from fluoro and hydroxy), phenyl, 5- or 6-membered heteroaryl, C₃₋₆-carbocyclyl and C₄₋₆-heterocyclyl (wherein the phenyl, 5- or 6-membered heteroaryl, C₃₋₆-carbocyclyl and C₄₋₆-heterocyclyl are optionally substituted with one or more substituents independently selected from halogen, C₁₋₃-alkyl and C₁₋₃-alkoxy);
R³ is a 5- or 6-membered heteroaryl group comprising at least one nitrogen atom, wherein the 5- or 6-membered heteroaryl group is optionally substituted with one or more substituents independently selected from C₁₋₃-alkyl, chloro, oxo, —CH₂OH, —CH₂OCH₃, —CHF₂ and —CH₂F;
R⁴ is hydrogen, methoxy or cyano;
R⁵ is hydrogen or fluoro;
R⁶, R⁷ and R⁸ are each independently selected from hydrogen and C₁₋₃-alkyl;
or a pharmaceutically acceptable salt thereof;
provided that the compound is not selected from
(R)—N-(6-methoxy-5-(4-methyl-1H-imidazol-1-yl)pyridin-2-yl)-4-methyl-2-phenyl-2,3,4,5-tetrahydropyrido[3,2-f][1,4]oxazepin-8-amine;
(R)-1-(8-(6-methoxy-5-(4-methyl-1H-imidazol-1-yl)pyridin-2-ylamino)-2-phenyl-2,3-dihydropyrido[3,2-f][1,4]oxazepin-4(5H)-yl)ethanone;
(R)—N-(6-Methoxy-5-(1-methyl-1H-pyrazol-4-yl)pyridin-2-yl)-4-methyl-2-phenyl-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(6-methylpyridin-2-yl)-2,3,4,5-tetrahydropyrido[3,2-f][1,4]oxazepin-8-amine;
(R)—N-(6-Methoxy-5-(1-methyl-1H-pyrazol-4-yl)pyridin-2-yl)-2-phenyl-2,3,4,5-tetrahydropyrido[3,2-f][1,4]oxazepin-8-amine; and
(R)—N-(5-(1-Methyl-1H-pyrazol-4-yl)pyridin-2-yl)-2-phenyl-2,3,4,5-tetrahydropyrido[3,2-f][1,4]oxazepin-8-amine.

In one embodiment, the invention relates to the compound of formula (I), or a pharmaceutically acceptable salt thereof, wherein:

R¹ is hydrogen, C₁₋₃-alkyl or —C(O)CH₃;
R² is C₂₋₄-alkyl (optionally substituted with one or more fluoro substituents), phenyl (optionally substituted with one or more substituents independently selected from halogen and C₁₋₃-alkoxy), 5- or 6-membered heteroaryl (optionally substituted with one or more substituents independently selected from halogen and C₁₋₃-alkyl), C₄₋₆-heterocyclyl, or C₃₋₄-carbocyclyl (optionally substituted with one or more halogen substituents);
R³ is a 6-membered heteroaryl group comprising at least one nitrogen atom, or a 5-membered heteroaryl group comprising at least two heteroatoms of which at least one is nitrogen, and wherein the 5- or 6-membered heteroaryl group is optionally substituted with C₁₋₃-alkyl or oxo;
R⁴ is hydrogen, methoxy or cyano;
R⁵ is hydrogen or fluoro; and
R⁶, R⁷ and R⁸ are each independently hydrogen or methyl.

In another embodiment, the invention relates to the compound of formula (I), or a pharmaceutically acceptable salt thereof, wherein R¹ is hydrogen, methyl or —C(O)CH₃. In yet another embodiment, R¹ is methyl.

In another embodiment, the invention relates to the compound of formula (I), or a pharmaceutically acceptable salt thereof, wherein R² is 2,2,2-trifluoroethyl, 3,3,3-trifluoropropyl, 2-(fluoromethyl)-3-fluoropropyl, phenyl (optionally substituted with chloro), pyridinyl (optionally substituted with fluoro or methyl), thiazolyl (optionally substituted with methyl), tetrahydrofuranyl, cyclopropyl or 3,3-difluorocyclobutyl.

In another embodiment, the invention relates to the compound of formula (I), or a pharmaceutically acceptable salt thereof, wherein R³ is pyrazolyl, imidazolyl, triazolyl, oxazolyl or thiazolyl, pyridinyl or pyrimidinyl, any of which is optionally substituted with methyl or oxo.

In another embodiment, the invention relates to the compound of formula (I), or a pharmaceutically acceptable salt thereof, wherein R⁴ is methoxy or cyano.

In another embodiment, the invention relates to the compound of formula (I), or a pharmaceutically acceptable salt thereof, wherein R⁵ is hydrogen.

In another embodiment, the invention relates to the compound of formula (I), or a pharmaceutically acceptable salt thereof, wherein each of R⁶, R⁷ and R⁸ independently is hydrogen or methyl.

In another embodiment, the invention relates to the compound of formula (I), or a pharmaceutically acceptable salt thereof, wherein:

R¹ is methyl;
R² is 2,2,2-trifluoroethyl, 3,3,3-trifluoropropyl, 2-(fluoromethyl)-3-fluoropropyl, phenyl (optionally substituted with chloro), pyridinyl (optionally substituted with fluoro or methyl), thiazolyl (optionally substituted with methyl), tetrahydrofuranyl, cyclopropyl or 3,3-difluorocyclobutyl;
R³ is pyrazolyl, imidazolyl, triazolyl, oxazolyl or thiazolyl, pyridinyl or pyrimidinyl, any of which is optionally substituted with methyl or oxo;
R⁴ is methoxy or cyano;
R⁵ is hydrogen; and
R⁶, R⁷ and R⁸ are each independently hydrogen or methyl.

In another embodiment, the invention relates to the compound of formula (I), or a pharmaceutically acceptable salt thereof, wherein:

R¹ is methyl;
R² is 2-(fluoromethyl)-3-fluoropropyl;
R³ is imidazolyl, which is substituted with methyl;
R⁴ is methoxy;
R⁵ is hydrogen; and
R⁶, R⁷ and R⁸ are each hydrogen.

In another embodiment, the invention relates to the compound of formula (I), or a pharmaceutically acceptable salt thereof, selected from the group consisting of:
4,6-Dimethyl-N-(5-(4-methyl-1H-imidazol-1-yl)pyridin-2-yl)-2-(2,2,2-trifluoroethyl)-2,3,4,5-tetrahydropyrido[3,2-f][1,4]oxazepin-8-amine;
N-(6-Methoxy-5-(1-methyl-1H-pyrazol-4-yl)pyridin-2-yl)-4,6-dimethyl-2-(2,2,2-trifluoroethyl)-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine;
N-(6-Methoxy-5-(4-methyl-1H-imidazol-1-yl)pyridin-2-yl)-4-methyl-2-(2,2,2-trifluoroethyl)-2,3,4,5-tetrahydropyrido[3,2-f][1,4]oxazepin-8-amine;
[6-Methoxy-5-(6-methyl-pyrimidin-4-yl)-pyridin-2-yl]-[6-methyl-8-(2,2,2-trifluoro-ethyl)-5,6,7,8-tetrahydro-9-oxa-1,6-diaza-benzocyclohepten-2-yl]-amine;
(2-Methoxy-2′-methyl-[3,4]bipyridinyl-6-yl)-[6-methyl-8-(2,2,2-trifluoro-ethyl)-5,6,7,8-tetrahydro-9-oxa-1,6-diaza-benzocyclohepten-2-yl]-amine;
N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine;
N-(6-Methoxy-5-(4-methyl-1H-imidazol-1-yl)pyridin-2-yl)-4-methyl-2-(4-methylthiazol-2-yl)-2,3,4,5-tetrahydropyrido[3,2-f][1,4]oxazepin-8-amine;
2-Cyclopropyl-N-(6-methoxy-5-(1-methyl-1H-pyrazol-4-yl)pyridin-2-yl)-4,6-dimethyl-2,3,4,5-tetrahydropyrido[3,2-f][1,4]oxazepin-8-amine;
(R)-4-Methyl-N-(5-(4-methyl-1H-imidazol-1-yl)pyridin-2-yl)-2-phenyl-2,3,4,5-tetrahydropyrido[3,2-f][1,4]oxazepin-8-amine;
(R)-4-Methyl-N-(5-(1-methyl-1H-pyrazol-4-yl)pyridin-2-yl)-2-phenyl-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(3-methylpyridin-2-yl)-2,3,4,5-tetrahydropyrido[3,2-f][1,4]oxazepin-8-amine;
2-Cyclopropyl-N-(6-methoxy-5-(1-methyl-1H-pyrazol-4-yl)pyridin-2-yl)-4,5-dimethyl-2,3,4,5-tetrahydropyrido[3,2-f][1,4]oxazepin-8-amine;
N-(6-Methoxy-5-(1-methyl-1H-pyrazol-4-yl)pyridin-2-yl)-4,6-dimethyl-2-(4-methylthiazol-2-yl)-2,3,4,5-tetrahydropyrido[3,2-f][1,4]oxazepin-8-amine;
2-(3-Chlorophenyl)-4-methyl-N-(5-(3-methyl-1H-1,2,4-triazol-1-yl)pyridin-2-yl)-2,3,4,5-tetrahydropyrido[3,2-f][1,4]oxazepin-8-amine;
2-(4-Chlorophenyl)-4-methyl-N-(5-(3-methyl-1H-1,2,4-triazol-1-yl)pyridin-2-yl)-2,3,4,5-tetrahydropyrido[3,2-f][1,4]oxazepin-8-amine;
N-(6-Methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine;
[6-Methoxy-5-(1-methyl-1H-pyrazol-4-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
[6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
N-(6-Methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine;
[8-(3-Fluoro-2-fluoromethyl-propyl)-6-methyl-5,6,7,8-tetrahydro-9-oxa-1,6-diaza-benzocyclohepten-2-yl]-[6-methoxy-5-(1-methyl-1H-pyrazol-4-yl)-pyridin-2-yl]-amine;
[8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine;
N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine;
2-(3,3-Difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(5-methylthiazol-2-yl)-2,3,4,5-tetrahydropyrido[3,2-f][1,4]oxazepin-8-amine;
N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(5-methylpyridin-2-yl)-2,3,4,5-tetrahydropyrido[3,2-f][1,4]oxazepin-8-amine;
N-(6-Methoxy-5-(1-methyl-1H-pyrazol-4-yl)pyridin-2-yl)-4-methyl-2-(4-methylpyridin-2-yl)-2,3,4,5-tetrahydropyrido[3,2-f][1,4]oxazepin-8-amine;
5-(2-Methoxy-6-(4-methyl-2-(2,2,2-trifluoroethyl)-2,3,4,5-tetrahydropyrido[3,2-f][1,4]oxazepin-8-ylamino)pyridin-3-yl)-1-methylpyridin-2(1H)-one;
(R)-5-(2-Methoxy-6-(4-methyl-2-phenyl-2,3,4,5-tetrahydropyrido[3,2-f][1,4]oxazepin-8-ylamino)pyridin-3-yl)-1-methylpyridin-2(1H)-one;
N-[6-Methoxy-5-(2-methylpyrimidin-4-yl)-2-pyridyl]-4-methyl-2-(2,2,2-trifluoroethyl)-3,5-dihydro-2H-pyrido[3,2-f][1,4]oxazepin-8-amine;
N-[6-Methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine;
6-[(2-Ethyl-4-methyl-3,5-dihydro-2H-pyrido[3,2-f][1,4]oxazepin-8-yl)amino]-3-(4-methylimidazol-1-yl)pyridine-2-carbonitrile;
(2R)—N-[6-Methoxy-5-(2-methyloxazol-5-yl)-2-pyridyl]-4-methyl-2-phenyl-3,5-dihydro-2H-pyrido[3,2-f][1,4]oxazepin-8-amine;
(2S)-2-(5-Fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine;
(2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine;
(2S)—N-[3-Fluoro-6-methoxy-5-(1-methylpyrazol-4-yl)-2-pyridyl]-4-methyl-2-(4-methylthiazol-2-yl)-3,5-dihydro-2H-pyrido[3,2-f][1,4]oxazepin-8-amine;
3-(4-Methylimidazol-1-yl)-6-[[(2S)-4-methyl-2-(4-methylthiazol-2-yl)-3,5-dihydro-2H-pyrido[3,2-f][1,4]oxazepin-8-yl]amino]pyridine-2-carbonitrile;
(2R)—N-[6-Methoxy-5-(1H-pyrazol-4-yl)-2-pyridyl]-4-methyl-2-phenyl-3,5-dihydro-2H-pyrido[3,2-f][1,4]oxazepin-8-amine; and
N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine.

In yet another embodiment, the invention relates to the compound of formula (I), or a pharmaceutically acceptable salt thereof, selected from the group consisting of:
(+)-N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine;
(−)-N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine;
(+)-N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine;
(−)-N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine;
(+)-[6-Methoxy-5-(1-methyl-1H-pyrazol-4-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
(−)-[6-Methoxy-5-(1-methyl-1H-pyrazol-4-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
(+)-[6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
(−)-[6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine;
(+)-[8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine;
(−)-[8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine;
(+)-N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-][1,4]oxazepin-8-amine;
(−)-N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine;
(−)-(2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine, isomer 1;
(−)-(2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine, isomer 2;
(−)-N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine; and
(+)-N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine.

In yet another embodiment, the invention relates to the compound of formula (I), or a pharmaceutically acceptable salt thereof, which is
(+)-[8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine.

In yet another embodiment, the invention relates to a compound of formula (I), or a pharmaceutically acceptable salt thereof, with the proviso that any of the specific Examples are individually disclaimed. For example, in a further embodiment the invention relates to a compound of formula (I), or a pharmaceutically acceptable salt thereof, with the proviso that the compound [8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine is disclaimed.

In a further embodiment, the invention relates to the compound 2-chloro-8-(3-fluoro-2-fluoromethyl-propyl)-6-methyl-5,6,7,8-tetrahydro-9-oxa-1,6-diaza-benzocycloheptene, or a salt thereof, which may be used as an intermediate in the preparation of a compound of formula (I).

In a second aspect, the invention relates to a pharmaceutical composition comprising the compound of formula (I), or a pharmaceutically acceptable salt thereof, in association with a pharmaceutically acceptable excipient, carrier or diluent.

In a third aspect, the invention relates to the compound of formula (I), or a pharmaceutically acceptable salt thereof, for use in treating or preventing an Aβ-related pathology.

In one embodiment, the invention relates to the compound of formula (I), or a pharmaceutically acceptable salt thereof, for use in treating or preventing Aβ-related pathologies selected from the group consisting of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy and cortical basal degeneration.

In a fourth aspect, the invention relates to a method of treating or preventing an Aβ-related pathology in a mammal, comprising administering to said mammal a therapeutically effective amount of the compound of formula (I), or a pharmaceutically acceptable salt thereof

In one embodiment, the invention relates to a method of treating or preventing in a mammal an Aβ-related pathology selected from the group consisting of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy and cortical basal degeneration, comprising administering to said mammal a therapeutically effective amount of the compound of formula (I), or a pharmaceutically acceptable salt thereof.

In a fifth aspect, the invention relates to a method of treating or preventing an Aβ-related pathology in a mammal, comprising administering to said mammal a therapeutically effective amount of a compound of formula (I), or a pharmaceutically acceptable salt thereof, and at least one cognitive enhancing agent, memory enhancing agent, acetyl choline esterase inhibitor, anti-inflammatory agent or atypical antipsychotic agent.

In a sixth aspect, the invention relates to the use of a compound of formula (I), or a pharmaceutically acceptable salt thereof, in the manufacture of a medicament for treatment or prevention of an Aβ-related pathology.

In one embodiment, the invention relates to the use of a compound of formula (I), or a pharmaceutically acceptable salt thereof, in the manufacture of a medicament for treatment or prevention of an Aβ-related pathology selected from the group consisting of Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy and cortical basal degeneration.

As used herein, “alkyl”, used alone or as a suffix or prefix, is intended to include both branched and straight chain saturated aliphatic hydrocarbon groups having from 1 to 4 carbon atoms or, if a specified number of carbon atoms is provided, then that specific number would be intended. For example “C₁₋₃-alkyl” denotes alkyl having 1, 2 or 3 carbon atoms. Examples of alkyl include methyl, ethyl, n-propyl, i-propyl, n-butyl, i-butyl, sec-butyl and tert-butyl.

The term “alkoxy”, unless stated otherwise, refers to radicals of the general formula —O—R, wherein R is an alkyl radical. For example “C₁₋₃-alkoxy” denotes alkoxy having 1, 2 or 3 carbon atoms. Examples of alkoxy include methoxy, ethoxy, n-propoxy and isopropoxy.

As used herein, “carbocyclyl”, used alone or as suffix or prefix, is intended to include cyclic saturated hydrocarbon groups from 3 to 6 ring carbon atoms. Examples of carbocyclyls include cyclopropyl, cyclobutyl, cyclopentyl and cyclohexyl.

As used herein, “heteroaryl” refers to a monocyclic heteroaromatic ring having 5 or 6 ring members and wherein at least one ring member is selected from sulfur, oxygen, and nitrogen. Examples include pyridyl, pyrimidinyl, pyrazinyl, pyridazinyl, triazinyl, furyl, thienyl, imidazolyl, thiazolyl, isothiazolyl, pyrryl, oxazolyl, isoxazolyl, pyrazolyl, triazolyl, tetrazolyl and thiadiazolyl.

As used herein, the term “optional” or “optionally” means that the subsequently described event or circumstance may but need not occur, and that the description includes instances where the event or circumstance occurs and instances where it does not.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “protecting group” means temporary substituents protecting a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been extensively reviewed (see, e.g. Jarowicki, K.; Kocienski, P. Perkin Trans. 1, 2001, issue 18, p. 2109).

As used herein, “pharmaceutically acceptable salts” refer to forms of the disclosed compounds, wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

A variety of compounds in the present invention may exist in particular geometric or stereoisomeric forms. The present invention takes into account all such compounds, including tautomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. All chiral, diastereomeric and racemic forms are intended, unless the specific stereochemistry or isomeric form is specifically indicated.

As used herein, “tautomer” means other structural isomers that exist in equilibrium resulting from the migration of a hydrogen atom. For example, keto-enol tautomerism occurs where the resulting compound has the properties of both a ketone and an unsaturated alcohol.

Compounds and pharmaceutically acceptable salts of the invention further include hydrates and solvates thereof.

Compounds and salts described in this specification may be isotopically-labelled compounds (or “radio-labelled”). In that instance, one or more atoms are replaced by an atom having an atomic mass or mass number different from the atomic mass or mass number typically found in nature (i.e., naturally occurring). Examples of suitable isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is used will depend on the specific application of that radio-labelled derivative. For example, for in vitro receptor labelling and competition assays, compounds that incorporate ³H, ¹⁴C, ⁸²Br, ¹²⁵I, ¹³¹I or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is ³H. In some embodiments, the radionuclide is ¹⁴C. In some embodiments, the radionuclide is ¹¹C. In some embodiments, the radionuclide is ¹⁸F.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In some embodiments, the present invention provides a compound of formula (I), or a pharmaceutically acceptable salt thereof, for the therapeutic treatment (including prophylactic treatment) of mammals including humans, which is normally formulated in accordance with standard pharmaceutical practice as a pharmaceutical composition.

The treatment of Aβ-related pathology defined herein may be applied as a sole therapy or may involve, in addition to the compound of the invention, conjoint treatment with conventional chemotherapy of value in treating one or more disease conditions referred to herein. Such conventional chemotherapy may include one or more of the following categories of agents: acetyl cholinesterase inhibitors, anti-inflammatory agents, cognitive and/or memory enhancing agents, or atypical antipsychotic agents. Cognitive enhancing agents, memory enhancing agents and acetyl choline esterase inhibitors include onepezil (ARICEPT), galantamine (REMINYL or RAZADYNE), rivastigmine (EXELON), tacrine (COGNEX) and memantine (NAMENDA, AXURA or EBIXA). Atypical antipsychotic agents include Olanzapine (marketed as ZYPREXA), Aripiprazole (marketed as ABILIFY), Risperidone (marketed as RISPERDAL), Quetiapine (marketed as SEROQUEL), Clozapine (marketed as CLOZARIL), Ziprasidone (marketed as GEODON) and Olanzapine/Fluoxetine (marketed as SYMBYAX).

Such conjoint treatment may be achieved by way of the simultaneous, sequential or separate dosing of the individual components of the treatment. Such combination products employ the compounds, or pharmaceutically acceptable salts thereof, of the invention.

In another aspect, the invention relates to a pharmaceutical composition comprising (i) a compound of formula (I), or a pharmaceutically acceptable salt thereof, (ii) an additional therapeutic agent, or a pharmaceutically acceptable salt thereof, and (iii) a pharmaceutically acceptable excipient, carrier or diluent.

In another aspect, the invention relates to a pharmaceutical composition comprising (i) a compound of formula (I), or a pharmaceutically acceptable salt thereof, (ii) at least one agent selected from the group consisting of acetyl cholinesterase inhibitors, anti-inflammatory agents, cognitive enhancing agents, memory enhancing agents, and atypical antipsychotic agents, and (iii) a pharmaceutically acceptable excipient, carrier or diluent.

Additional conventional chemotherapy may include one or more of the following categories of agents:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine and venlafaxine.
(ii) atypical antipsychotics such as quetiapine.
(iii) antipsychotics such as amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine and ziprasidone.
(iv) anxiolytics such as alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam and zolazepam.
(v) anticonvulsants such as carbamazepine, valproate, lamotrogine and gabapentin.
(vi) Alzheimer's therapies such as donepezil, memantine and tacrine.
(vii) Parkinson's therapies such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase.
(viii) migraine therapies such as almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan and zomitriptan.
(ix) stroke therapies such as abciximab, activase, NXY-059, citicoline, crobenetine, desmoteplase, repinotan and traxoprodil.
(x) urinary incontinence therapies such as darafenacin, falvoxate, oxybutynin, propiverine, robalzotan, solifenacin and tolterodine.
(xi) neuropathic pain therapies such as gabapentin, lidoderm and pregablin.
(xii) nociceptive pain therapies such as celecoxib, etoricoxib, lumiracoxib, rofecoxib, valdecoxib, diclofenac, loxoprofen, naproxen and paracetamol.
(xiii) insomnia therapies such as agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon and zolpidem.
(xiv) mood stabilizers such as carbamazepine, divalproex, gabapentin, lamotrigine, lithium, olanzapine, quetiapine, valproate, valproic acid and verapamil.

### Preparation of Compounds

Preparation of the compounds of the present invention will be illustrated below.

In each of the following preparation methods, when a defined group changes under reaction conditions or is not suitable for carrying out the method, the preparation can be easily carried out by subjecting the group to a procedure conventionally employed in organic synthetic chemistry, such as protection and/or deprotection of a functional group (for example, see Protection Groups in Organic Synthesis, T. W. Green, Wiley & Sons Inc. (1999)).

Where necessary, the order of reaction process steps such as introduction of substituents can be altered. Solvent, temperature, pressure and other reaction conditions may readily be selected by the skilled person. Starting materials are commercially available or readily prepared by one skilled in the art. Compounds of formula (I) can be prepared, for example, using the Methods of Preparation 1 and 2. In the methods of preparation below, PG represents a protective group or a substituent. PG is can be replaced or exchanged prior to, during or immediately following the process mentioned below.

### Method of Preparation 1

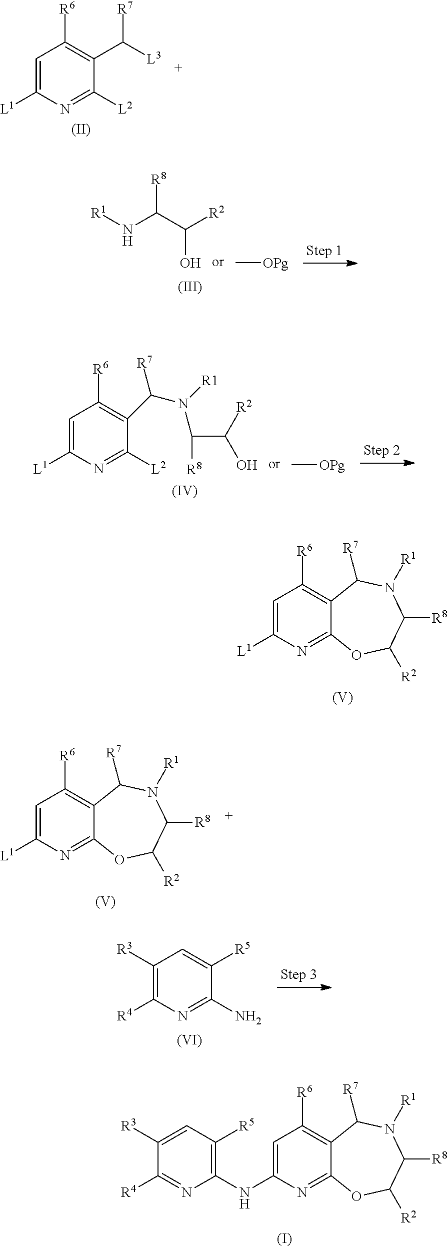

wherein L¹ and L² are halogen; L³ is Cl, Br, I or OS(O)₂CH₃;
R¹, R², R³, R⁴, R⁵, R⁶, R⁷ and R⁸ are as defined in claim 1.

### Step 1

A compound of formula (IV) is obtained by reacting a compound of formula (II) (see preparation of intermediates below) with a compound of formula (III) as depicted above. The reaction is carried out in a suitable solvent (such as EtOH, MeOH, DMF, dioxane, THF or 2-methyl-THF), optionally in the presence of a base such as a tertiary amine (e.g., triethylamine, diisopropylamine) or an inorganic base (e.g., potassium carbonate, sodium carbonate, cesium carbonate, sodium tert-butoxide) at a temperature between −78° C. and 150° C. Addition of a catalytic amount of potassium iodide can be advantageous.

Alternatively, a reductive amination can be performed on an intermediate of formula (XVI) (see preparation of intermediates below), in the presence of an amine of formula (III) to form a compound of formula (IV). The reaction is performed in a suitable solvent (such as MeOH, 1,2-dichloroethane, THF or MeCN), sometimes in the presence of a catalyst (such as acetic acid, boric acid, p-toluenesulfonic acid monohydrate or benzoic acid). Examples of the reductive reagent include sodium cyanoborohydride, sodium triacetoxyborohydride and decaborane. The reaction is typically run under inert atmosphere at temperatures between 0 and 100° C.

### Step 2

A compound of formula (IV) is converted to a compound of formula (V) via an intramolecular ring closure reaction. The reaction is generally performed in the presence of a base and in a suitable solvent (ether, THF, 2-methyl-THF, dioxane, DMF and the like). Examples of base include metal hydride (such as potassium hydride or sodium hydride), inorganic base (such as lithium hydroxide, sodium hydroxide, potassium hydroxide, sodium hydrogen carbonate, hydrogen carbonate, cesium carbonate, sodium ethoxide or sodium tert-butoxide) and organic base (such as triethylamine, diisopropylamine or pyridine). The reaction temperature is, for example, about −78° C. to about 150° C.

Steps 1 and 2 can also be performed in a one-pot procedure to give a compound of formula (V) by reacting a compound of formula (II) with a compound of formula (III) (with the alcohol functionality unprotected) in the presence of a base, such as sodium tert-butoxide in an inert solvent such as THF or 2-methyl-THF. The typical starting temperature is −78° C. for the first step and then the temperature is increased to ambient temperature for the ring closure.

### Step 3

A compound of formula (V) is reacted with a nucleophile of formula (VI) under thermal heating or under cross-coupling conditions to form a compound of formula (I).

Heating a compound of formula (V) in the presence of a suitable nucleophile of formula (VI) affords a compound of formula (I). The reaction is generally performed in the presence of a base and in a suitable solvent (ether, THF, 2-methyl-THF, dioxane, DMF and the like). Examples of base include metal hydride (such as potassium hydride or sodium hydride), inorganic base (such as lithium hydroxide, sodium hydroxide, potassium hydroxide, sodium hydrogen carbonate, hydrogen carbonate, cesium carbonate or sodium ethoxide) and organic base (such as triethylamine, diisopropylamine or pyridine). The reaction temperature is, for example, about −50° C. to about 150° C.

A cross-coupling reaction is an alternative method for converting a compound of formula (V) into a compound of formula (I). A compound of formula (V) and of formula (VI) are heated in the presence of a catalyst such as Pd(OAc)₂, Pd(dba)₂, Pd₂(dba)₃ and a ligand such as BINAP, dppf, 2-(dicyclohexylphosphino)biphenyl, 2-(di-tert-butylphosphino)biphenyl or Xantphos, a suitable base (such as potassium tert-butoxide, sodium tert-butoxide, sodium-pentoxide or cesium carbonate) in a suitable solvent such as 1,4-dioxane (see for examples Accounts of Chemical Research, 2002, 35, 717; and J. Am. Chem. Soc. 2003, 125, 6653).

### Method of Preparation 2

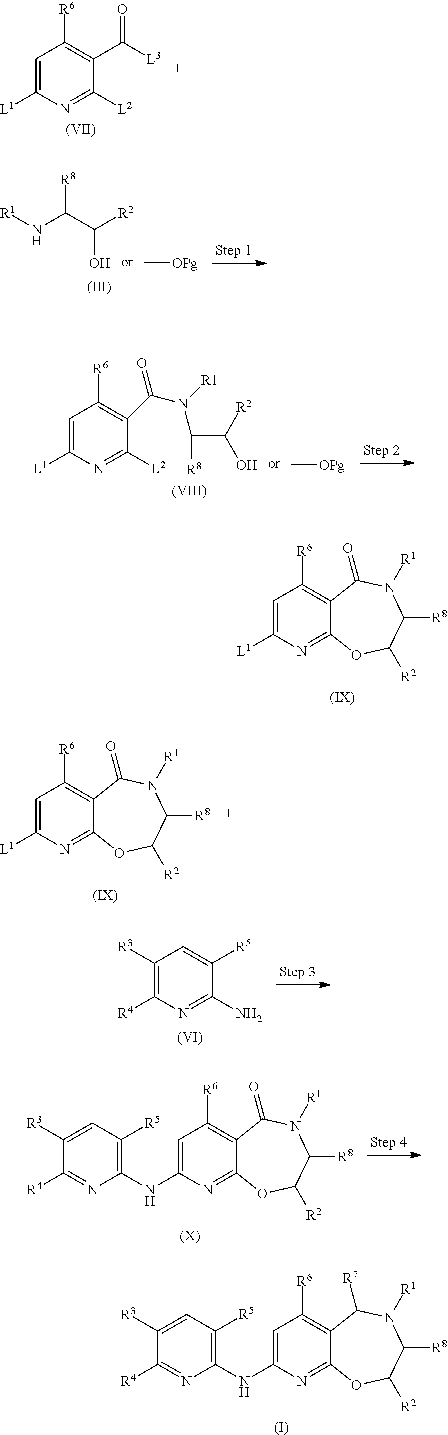

wherein L¹ and L² are halogen; L³ is Cl or OH;
R¹, R², R³, R⁴, R⁵, R⁶ and R⁸ are as defined in claim 1;

### R⁷ is H.

### Step 1

In this step, a carboxylic acid (derivative) of formula (VII) and an amine of formula (III) are subjected to a dehydrative condensation to give a compound of formula (VIII). The dehydrative condensation is performed by a method known per se, for example, a method using a condensation agent or a method using a reactive derivative.

Examples of the condensation reagent used include dicyclohexylcarbodiimide, diisopropylcarbodiimide and O-benzotriazol-1-yl-N,N,N′,N′-tetra-methyluronium hexafluorophosphate. They may be used alone or in combination of additives (e.g., N-hydroxysuccinimide, 1-hydroxybenzotriazol). The reaction above is generally performed in a suitable solvent (e.g., DCM, DMF, THF, pyridine) and an appropriate base can also be present (e.g., triethylamine, diisopropylmethylamine, sodium hydroxide).

Alternatively, reactive derivatives such as acid halides and active esters can be reacted with an amine of formula (III) to form a compound of formula (VIII). The reactive derivatives are prepared under standard conditions known by a person skilled in the art. A carboxylic acid of formula (VII) is converted to an acid halide using reagents such as thionyl chloride, oxalyl chloride and phosphorus trichloride, either neat or in the presence of a suitable solvent (DCM, THF, dioxane and the like). The reactive derivative of formula (VII) and the amine of formula (III) are mixed generally in the presence of a base (such as triethylamine, diisopropylamine or sodium hydroxide) in a suitable solvent (THF, DCM, dioxane and the like) at an appropriate temperature (about −50° C. to the boiling point of the solvent) to afford a compound of formula (VIII).

### Step 2

A compound of formula (VIII) is converted to a compound of formula (IX) via an intramolecular ring closure reaction. The reaction is generally performed in presence of a base and in a suitable solvent (ether, THF, 2-methyl-THF, dioxane, DMF and the like). Examples of base include metal hydride (such as potassium hydride or sodium hydride), inorganic base (such as lithium hydroxide, sodium hydroxide, potassium hydroxide, sodium hydrogen carbonate, hydrogen carbonate, sodium ethoxide or sodium tert-butoxide) and organic base (such as triethylamine, diisopropylamine or pyridine). The reaction temperature is, for example, about −50° C. to about 150° C.

### Step 3

A compound of formula (IX) is reacted with a nucleophile of formula (VI) under thermal heating or under cross-coupling conditions to form a compound of formula (X) as described in Method of Preparation 1, step 3.

### Step 4

A compound of formula (I) can be prepared by reducing the amide functionality in a compound of formula (X). A compound of formula (X) is allowed to react with a reducing agent (such as lithium aluminium hydride, borane or carbonylhydrotris(triphenylphosphine)rhodium(I) in combination with diphenylsilane) in a suitable solvent (such as THF, 2-methyl-THF or diethyl ether) at a temperature between about −50° C. and the boiling point of the solvent.

### Preparation of Intermediates

### Preparation of Intermediate (II)

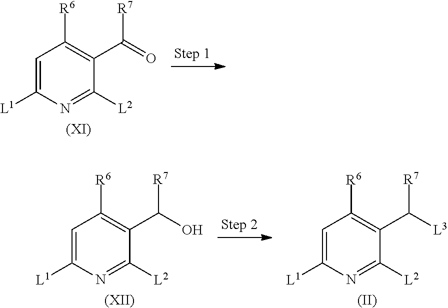

wherein L¹ and L² are halogen; L³ is Cl or Br;
R⁶ and R⁷ are as defined in claim 1.

### Step 1

A compound of formula (XII) can be prepared by treating a compound of formula (XI) with a reducing agent such as sodium borohydride in a solvent such as methanol, or lithium borohydride in a solvent such as THF, at 0° C. to ambient temperature.

### Step 2

A compound of formula (II) can be prepared by treating a compound of formula (XII) with a halogenating reagent such as N-bromosuccinimide or N-chlorosuccinimide in the presence of triphenylphosphine in a solvent such as dichloromethane or 1,2-dichloroethane at 0° C. to ambient temperature.

### General Methods

All solvents used were of analytical grade and commercially available anhydrous solvents were routinely used for reactions. Starting materials used were available from commercial sources, or prepared according to literature procedures.

Microwave heating was performed in a Creator, Initiator or Smith Synthesizer Single-mode microwave cavity producing continuous irradiation at 2450 MHz alternatively in a CEM Discover LabMate instrument. It is understood that microwaves can be used for the heating of reaction mixtures.

NMR spectroscopy was performed on a Bruker DPX400 NMR spectrometer operating at 400 MHz for ¹H, 376 MHz for ¹⁹F, and 100 MHz for ¹³C, equipped with a 4-nucleus probe-head with Z-gradients. Alternatively, NMR spectroscopy was performed on a Bruker 500 MHz Avance III NMR spectrometer, operating at 500 MHz for ¹H, 125 MHz for ¹³C, and 50 MHz for ¹⁵N equipped with a 5 mm TCI cryogenically cooled probe-head with Z-gradients. Alternatively, NMR spectroscopy was performed on a Bruker DRX600 NMR spectrometer, operating at 600 MHz for ¹H, 150 MHz for ¹³C and 60 MHz for ¹⁵N, equipped with a 5 mm TXI probe-head with Z-gradients. Alternatively, NMR spectroscopy was performed on a Varian Mercury Plus 400 NMR Spectrometer equipped with a Varian 400 ATB PFG probe, operating at 400 MHz for ¹H and 100 MHz for ¹³C.

The following reference signals were used: the middle line of (CD₃)₂SO δ 2.50 (¹H), δ 39.51 (¹³C); the middle line of CD₃OD δ 3.31 (¹H) or δ 49.15 (¹³C); CDCl₃ δ 7.26 (¹H) and the middle line of CDCl₃ δ 77.16 (¹³C); if the solvent contained 0.03% to 0.05% v/v tetramethylsilane, δ 0.00 (¹H and ¹³C); unless otherwise indicated.

LC-MS analyses were performed on an LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was equipped with an electrospray ion source (ES) operated in positive and negative ion mode. The column used was a Xbridge C18, 3.0×50 mm, 5 μm which was run at a flow rate of 2 ml/min. Alternatively, HPLCMS analyses were performed on a Waters Acquity HPLC system consisting of an Acquity Autosampler, Acquity Sample Organizer, Acquity Column Manager, Acquity Binary Solvent Manager, Acquity HPLC PDA detector and a Waters 3100 Mass Spectrometer. The mass spectrometer was equipped with an ESCi ion source, Electrospray ionisation (ES) and/or Atmospheric Pressure Chemical ionisation (APCI), operated in positive and negative ion mode. Separation was performed on an Acquity column, HPLC BEH, C18 2.1×50 mm, 1.7 μm run at a flow rate of 0.5 mL/min.

Alternatively, mass spectra were recorded on a Waters MS consisting of an Alliance 2795 (LC) and Waters Micromass ZQ detector at 120° C. The mass spectrometer was equipped with an electrospray ion source (ES) operated in a positive or negative ion mode. The mass spectrometer was scanned between m/z 100-1000 with a scan time of 0.3 s.

Typical mobile phase systems for LCMS consisted of
Mobile phase A: 10 mM NH₄OAc in 5% CH₃OH) and mobile phase B: CH₃OH or
Mobile phase A: 0.1% NH₃ in MilliQ and mobile phase B: CH₃OH.

A linear gradient from 100% A to 100% B was typically applied.

Preparative chromatography was run on a Waters FractionLynx system with an Autosampler combined Automated Fraction Collector (Waters 2767), Gradient Pump (Waters 2525), Column Switch (Waters CFO) and PDA (Waters 2996). Column; XBridge® Prep C8 10 μm OBD™ 19×250 mm, with guard column; XTerra® Prep MS C8 10 μm 19×10 mm Cartridge. Flow rate 20 mL/min. The PDA was scanned from 218-400 nm. UV triggering determined the fraction collection. Linear gradient of B was applied.

Typical mobile phase systems are:
Mobil phase A: 95% 0.1 M NH₄OAc in MilliQ water and 5% CH₃OH in mobile phase B: 100% CH₃OH; or
Mobile phase A: 0.2% formic acid in MilliQ water and mobile phase B: 100% CH₃OH; or
Mobile phase A: 0.2% NH₃ in MilliQ water and mobile phase B: 100% CH₃OH.

Alternatively, preparative chromatography was performed on either a Waters Prep LC 4000 System using a Waters 2487 Diode Array or on a Waters LC Module 1 plus. The column used was either a Waters XTerra Prep C₁₈, 5 μm, 30×100 mm (flow rate 40 mL/min) or a Phenomenex Luna C₁₈, 5 μm, 21.6×250 mm (flow rate 20 mL/min). Narrow gradients with acetonitrile/water, with the water containing either 0.1% trifluoroacetic acid or 10 mM ammonium acetate, were used to elute the compound in a total run time between 20-30 min.

Purity with mass analyses were performed on an Agilent HP1100 system consisting of a G1379A Micro Vacuum Degasser, a G1312A Binary Pump, a G1367 A Well-Plate Autosampler, a G1316A Thermostatted Column Compartment, a G1315C Diode Array Detector and a G6120A mass spectrometer, equipped with a G1978A multimode ion source. The mass spectrometer was set to electrospray ionization (ES) and operated in positive and negative ion mode. The column used was a Kinetex C18 4.6×50, 2.6 μm or an XBridge C18 3.0×100 mm, 3 μm run at a flow rate of 2.0 mL/min. A linear gradient was used for both the blank and the sample, starting at 100% A (A: 10 mM NH₄OAc in 5% CH₃CN) and ending at 100% B (B: CH₃CN). The PDA was scanned from 210-350 nm. UV triggering determined the fraction collection.

Preparative chromatography for chiral separation was run on a Berger Multigram II system. The methods used are described in the examples.

The optical rotation was run on an Agilent HPLC system with a PDR Chiral detector Column: Chiralcel OD-H, 4.6*250 mm; 5 μm. Mobile phase: 100% EtOH. Flowrate: 1 mL/min.

Flash chromatography was performed on a Combi Flash® Companion™ using RediSep™ normal-phase flash columns or using Merck Silica gel 60 (0.040-0.063 mm). Typical solvents used for flash chromatography were mixtures of chloroform/MeOH, DCM/MeOH, heptane/EtOAc, chloroform/MeOH/ammonia (aq.) and DCM/MeOH/NH₃ (aq.).

Elemental Analysis for C, H and N composition was performed using a Costech Instrument Elemental Combustion System ECS4010 with a helium flow of 100 mL/min (14 psi), oxygen 20 mL/min (10 psi), air 25 psi and purge of 50 mL/min. The reported analyses are the best of at least two runs.

Compounds have been named using CambridgeSoft MedChem ELN v2.1, ChemDraw Ultra 7.0 software featuring AutoNom version 2.2 (licensed from Beilstein Informationssysteme) or according to IUPAC rules.

### Abbreviations

aq. Aqueous
Ar Argon (or Argon atmosphere)
br broadened
BINAP 2,2′-bis(diphenylphosphino)-1,1′-binaphthyl
CI chemical ionization
δ chemical shift in parts per million (ppm) downfield from the standard
d doublet
DAST 4-[4-[[4-chloro-3-(trifluoromethyl)phenyl]carbamoylamino]-3-fluoro-phenoxy]-N-methyl-pyridine-2-carboxamide
DEA diethylamine
DCM dichloromethane
DIPEA N,N-diisopropylethylamine
DME 1,2-dimethoxyethane
DMF N,N-dimethylformamide
DMSO dimethyl sulfoxide
dppf 1,1′-bis(diphenylphosphino)ferrocene
EI electron impact
eq equivalents
ES electro-spray
ELS electron light scattering
Et₂O diethyl ether
EtOAc ethyl acetate
EtOH ethanol
h hour(s)
HCl hydrochloride acid
HPLC high performance liquid chromatography
IBX 2-Iodoxybenzoic acid
LC liquid chromatography
m multiplet
mCPBA 3-chlorobenzenecarboperoxoic acid
Me methyl
MeCN acetonitrile
MeOH methanol
min minute(s)
NBS N-bromosuccinimide
NMR nuclear magnetic resonance
MS mass spectroscopy
MTBE methyl tert-butyl ether
o.n. over-night
Pd 118 dichloro[1,1′bis(di-tert-butylphosphino)]ferrocene palladium (II)
Pd₂(dba)₃ tris(dibenzylideneaceton)dipalladium
Pd(OAc)₂ palladium(II) acetate
PDA photodiode array detector
PPh₃ triphenylphosphine
prep. preparative
q quartet
quin quintet
r.t. room temperature (ca. 21-25° C.)
s singlet
sat. saturated
SFC supercritical fluid chromatography
t triplet
TEA triethylamine
TFA trifluoroacetic acid
THF tetrahydrofuran
UV ultra violet
Xantphos 4,5-bis(diphenylphosphino)-9,9-dimethylxanthene

### EXAMPLES

Below follows a number of non-limiting examples of compounds of the invention.

### Example 1

### 4,6-Dimethyl-N-(5-(4-methyl-1H-imidazol-1-yl)pyridin-2-yl)-2-(2,2,2-trifluoroethyl)-2,3,4,5-tetrahydropyrido[3,2-f][1,4]oxazepin-8-amine

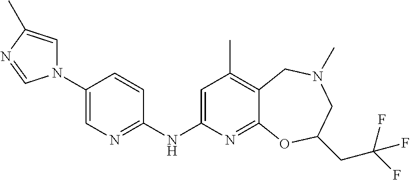

To 8-chloro-4,6-dimethyl-2-(2,2,2-trifluoroethyl)-2,3,4,5-tetrahydropyrido[3,2-f][1,4]oxazepine (Example 1d, 300 mg, 0.51 mmol) in DME (4 mL) were added 5-(4-methyl-1H-imidazol-1-yl)pyridin-2-ylamine (Example 1f, 89 mg, 0.51 mmol), cesium carbonate (249 mg, 0.76 mmol), 2-(dicyclohexylphosphino)biphenyl (17.8 mg, 0.05 mmol) and palladium acetate (11.4 mg, 0.05 mmol). The reaction was heated to 110° C. for 3×90 min. The solids were filtered off and washed with DCM and isopropanol and discarded. The solvents were evaporated and the crude product was purified using first flash chromatography, 0-7% MeOH in DCM and then preparative HPLC yielding the title compound (34 mg, 15%). ¹H NMR (400 MHz, CDCl₃) δ ppm 2.28-2.38 (m, 7H) 2.46 (s, 3H) 2.71 (ddd, 1H) 2.94-2.99 (m, 2H) 3.61-3.87 (m, 2H) 4.34-4.44 (m, 1H) 6.94 (br. s., 1H) 7.08 (s, 1H) 7.48 (s, 1H) 7.59 (dd, 1H) 7.68 (br. s., 1H) 7.79 (d, 1H) 8.29 (d, 1H). MS m/z 433.2 [M+H]⁺.

### Example 1a

### (2,6-Dichloro-4-methylpyridin-3-yl)methanol

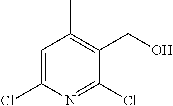

2,6-Dichloro-4-methylnicotinic acid (CAS 62774-90-7, 5 g, 24.27 mmol) in THF (25 mL) was treated with borane-THF complex 1M (44.9 mL, 44.9 mmol) at 0° C. The mixture was allowed to warm up to r.t. o.n. Saturated NaHCO₃ solution (10 mL) was added and stirred for 1 h at r.t. The solids were removed by filtration. The organic solvent was removed in vacuo. The crude product in the aqueous phase was partitioned between more water and DCM. The organic phase was separated and dried over MgSO₄. The solvent was evaporated yielding the title compound (4.22 g, 90%). 1H NMR (500 MHz, CDCl₃) δ ppm 2.49 (d, 3H) 4.83 (s, 2H) 7.15 (s, 1H). MS m/z 191.9 [M+H]⁺.

### Example 1b

### 3-(Bromomethyl)-2,6-dichloro-4-methylpyridine

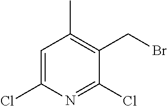

To (2,6-dichloro-4-methylpyridin-3-yl)methanol (Example 1a, 4.1 g, 21.35 mmol) in DCM (25 mL) was added PBr₃ (2.01 mL, 21.3 mmol) in DCM (1.5 mL). The reaction was heated to reflux for 15 min and then allowed to regain r.t. Sat. NaHCO₃ (10 mL) was added. The organic phase was separated, dried over MgSO₄ and then the solvent was evaporated yielding the title compound (5.12 g, 94%). ¹H NMR (500 MHz, CDCl₃) δ ppm 2.47 (s, 3H) 4.58 (s, 2H) 7.15 (s, 1H). MS m/z 255.8 [M+H]⁺.

### Example 1c

### 1-(((2,6-Dichloro-4-methylpyridin-3-yl)methyl)(methyl)amino)-4,4,4-trifluorobutan-2-ol

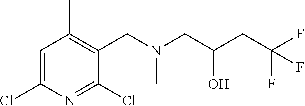

To 4,4,4-trifluoro-1-(methylamino)butan-2-ol (Example 1e, 1 g, 6.36 mmol) and TEA (0.89 mL, 6.36 mmol) in MeCN (10 mL) was added 3-(bromomethyl)-2,6-dichloro-4-methylpyridine (Example 1b, 1.46 g, 5.73 mmol) in MeCN (7 mL). The reaction was stirred at r.t. for 20 min. The solvent was evaporated. The crude product was partitioned between water and DCM. The organic phase was dried over MgSO₄ and then the solvent was removed yielding the title compound (1.8 g, 95%). MS m/z 333.0 [M+H]⁺.

### Example 1d

### 8-Chloro-4,6-dimethyl-2-(2,2,2-trifluoroethyl)-2,3,4,5-tetrahydropyrido[3,2-f][1,4]oxazepine

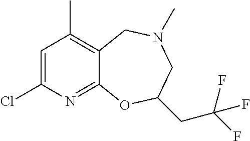

To sodium hydride (0.283 g, 7.07 mmol) in THF (10 mL) was added 1-(((2,6-dichloro-4-methylpyridin-3-yl)methyl)(methyl)amino)-4,4,4-trifluorobutan-2-ol (Example 1c, 1.8 g, 5.44 mmol) in THF (10 mL). The reaction was heated to 50° C. for 20 min. The reaction was quenched with water and the solvents were evaporated. A 50/50 mixture of two products was observed. The crude products were purified on silica, 0-7% MeOH in DCM but without success of separation. The mixture was used as such. MS m/z 295.6 [M+H]

### Example 1e

### 4,4,4-Trifluoro-1-(methylamino)butan-2-ol

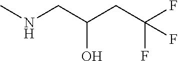

2-(2,2,2-Trifluoroethyl)oxirane (CAS 407-12-5, 1.487 g, 11.79 mmol) and 33% methanamine in EtOH (19.0 mL, 153.3 mmol) were heated to 80° C. in a microwave apparatus for 30 min. The solvent was evaporated to give 4,4,4-trifluoro-1-(methylamino)butan-2-ol (1.70 g, 92%). ¹H NMR (400 MHz, CDCl₃) δ ppm 2.12-2.42 (m, 4H), 2.46 (s, 3H), 2.54 (dd, 1H), 2.74 (dd, 1H), 3.94-4.03 (m, 1H).

### Example 1f

### 5-(4-Methyl-1H-imidazol-1-yl)pyridin-2-ylamine

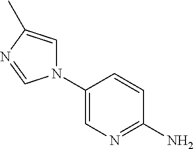

CuI (381 mg, 2 mmol), L-proline (461 mg, 4 mmol) and potassium carbonate (2.76 g, 20 mmol) were added to a solution of 5-iodo-pyridin-2-ylamine (CAS 20511-12-0, 2.2 g, 10 mmol) and 4-methyl-1H-imidazole (CAS 822-36-6, 1.15 g, 14 mmol) in anhydrous DMSO (20 mL). The reaction mixture was heated at 90° C. o.n., cooled to r.t. and diluted with EtOAc (50 mL) and filtered through a pad of Celite. The filtrate was concentrated under reduced pressure and the residue was purified first by flash column chromatography using a gradient of 1 to 5% MeOH in DCM, followed by prep. HPLC purification to afford the title compound (650 mg, 37%). ¹H NMR (400 MHz, CDCl₃) δ ppm 2.29 (s, 3H) 4.62 (br. s., 2H) 6.57 (d, 1H) 6.87 (s, 1H) 7.42 (dd, 1H) 7.59 (s, 1H) 8.12 (d, 1 H). MS (ES) m/z 175.1 [M+H]⁺.

### Example 2

### N-(6-Methoxy-5-(1-methyl-1H-pyrazol-4-yl)pyridin-2-yl)-4,6-dimethyl-2-(2,2,2-trifluoroethyl)-2,3,4,5-tetrahydropyrido[3,2-f][1,4]oxazepin-8-amine

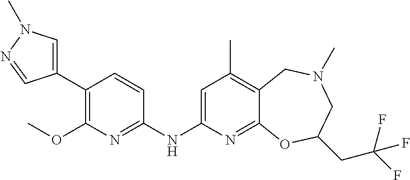

6-Methoxy-5-(1-methyl-1H-pyrazol-4-yl)pyridin-2-ylamine (Example 2a, 104 mg, 0.51 mmol), 8-chloro-4,6-dimethyl-2-(2,2,2-trifluoroethyl)-2,3,4,5-tetrahydropyrido[3,2-f][1,4]oxazepine (Example 1d, 150 mg, 0.51 mmol), sodium tert-butoxide (73.4 mg, 0.76 mmol), rac-2,2′-bis(diphenylphosphino)-1,1′-binaphthyl (22.2 mg, 0.04 mmol) and tris(dibenzylideneacetone)dipalladium(0) (32.6 mg, 0.04 mmol) were added to a Radley tube followed by toluene (6 mL). The reaction mixture was flushed with argon and the mixture was heated to 100° C. and stirred o.n. The solids were filtered off and washed with DCM. The crude product was purified on prep. HPLC yielding the title compound (23.0 mg, 9.8%). ¹H NMR (500 MHz, DMSO-d₆) δ ppm 2.28 (s, 3H) 2.32 (s, 3H) 2.58-2.71 (m, 2H) 2.82-2.90 (m, 2H) 3.48-3.54 (m, 1H) 3.75-3.80 (m, 1H) 3.85 (s, 3H) 4.00 (s, 3H) 4.22-4.29 (m, 1H) 7.14 (d, 1H) 7.58 (s, 1H) 7.82 (d, 1H) 7.84 (d, 1H) 8.02 (s, 1H) 9.45 (s, 1H). MS m/z 463.2 [M+H]⁺.

### Example 2a

### 6-Methoxy-5-(1-methyl-1H-pyrazol-4-yl)pyridin-2-ylamine

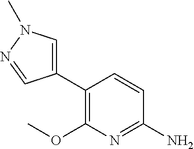

### Method 1:

To MeCN (20 mL) and water (10.0 mL) were added 5-bromo-6-methoxypyridin-2-amine (CAS 1211533-83-3, 2.2 g, 10.8 mmol), 1-methyl-4-(4,4,5,5-tetramethyl-1,3,2-dioxaborolan-2-yl)-1H pyrazole (CAS 761446-44-0, 2.93 g, 14.1 mmol) and Suzuki mix s-phos [(dicyclohexyl(2′,6′-dimethoxybiphenyl-2-yl)phosphine (329 mg, 0.8 mmol), potassium carbonate (3025 mg, 21.9 mmol) and palladium(II) acetate (123 mg, 0.55 mmol)]. The reaction was heated to reflux for 1 h. The mixture was cooled to r.t. and the solvents were evaporated. DCM (10 mL) was added and a precipitate was formed. 1.5 g of product was filtered off and washed with ether. Another 80 mg of product precipitated o.n. in DCM. The remaining product in the mother-liquor was purified using flash chromatography, 0-100% EtOAc in heptane yielding in total 1.67 g (76%) of the title compound. ¹H NMR (400 MHz, DMSO-d₆) δ ppm 3.82 (s, 3H) 3.84 (s, 3H) 5.85 (s, 2H) 6.05 (d, 1H) 7.59 (d, 1H) 7.70 (d, 1H) 7.86 (s, 1H). MS m/z 205.6 [M+H]⁺.

### Method 2:

1-Methyl-4-(4,4,5,5-tetramethyl-1,3,2-dioxaborolan-2-yl)-1H-pyrazole (CAS 761446-44-0, 205 mg, 0.99 mmol), potassium carbonate (340 mg, 2.46 mmol) and Pd 118 (32.1 mg, 0.05 mmol) were added to 5-bromo-6-methoxypyridin-2-amine (CAS 1211533-83-3, 200 mg, 0.99 mmol) in dioxane (3 mL). Water (0.5 mL) was added and the reaction was heated to 110° C. for 45 min in the microwave reactor. The solids were filtered off and washed with DCM. The solvent was evaporated and the crude product was purified on silica column chromatography using a gradient of 0-5% MeOH in DCM yielding the title compound (147 mg, 73%). ¹H NMR (500 MHz, DMSO-d₆) δ ppm 3.83 (s, 3H) 3.86 (s, 3H) 5.84 (s, 2H) 6.06 (d, 1H) 7.59 (d, 1H) 7.71 (s, 1H) 7.87 (s, 1H). MS m/z 205 [M+H]⁺.

### Example 3

### N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine

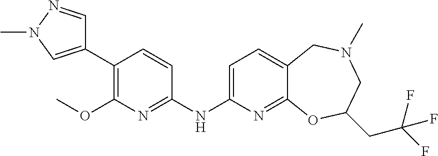

8-Chloro-4-methyl-2-(2,2,2-trifluoroethyl)-2,3,4,5-tetrahydropyrido[3,2-f][1,4]oxazepine (Example 3b, 60% purity, 314 mg, 0.67 mmol), 6-methoxy-5-(1-methyl-1H-pyrazol-4-yl)pyridin-2-ylamine (Example 2a, 137 mg, 0.67 mmol), palladium acetate (15.07 mg, 0.07 mmol), 2-(dicyclohexylphosphino)biphenyl (23.5 mg, 0.07 mmol) and cesium carbonate (328 mg, 1.01 mmol) were placed in a microwave vial. The vial was capped and flushed with argon. DME (3 mL) was added via a syringe and the resulting mixture was heated to 100° C. in a microwave apparatus for 2 h. The reaction mixture was diluted with dichloromethane, filtered and concentrated. The residue was purified first by column chromatography using silica stationary phase and gradient elution with increasing concentration of methanol, from 0 to 8%, in dichloromethane, and then by reversed phase HPLC to give the title compound (102 mg, 34%). ¹H NMR (500 MHz, CDCl₃) δ ppm 2.35-2.44 (m, 1H), 2.46 (s, 3H), 2.66-2.80 (m, 1H), 2.91-3.05 (m, 2H), 3.60 (d, 1H), 3.76 (d, 1H), 3.95 (s, 3H), 4.06 (s, 3H), 4.40-4.48 (m, 1H), 6.79 (d, 1H), 7.14 (s, 1H), 7.46 (d, 1H), 7.60 (d, 1 H), 7.71 (d, 1H), 7.79 (s, 1H), 7.82 (s, 1H). MS m/z 449.2 [M+H]⁺.

### Example 3a

### 1-(((2,6-Dichloropyridin-3-yl)methyl)(methyl)amino)-4,4,4-trifluorobutan-2-ol

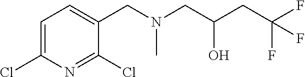

4,4,4-Trifluoro-1-(methylamino)butan-2-ol (Example 1e, 0.696 g, 4.43 mmol) and TEA (0.617 mL, 4.43 mmol) was added to a solution of 3-(bromomethyl)-2,6-dichloropyridine (CAS 58596-59-1, 1.067 g, 4.43 mmol) in MeCN (10 mL). The resulting mixture was stirred at r.t. for 2 h. The solvent was evaporated and the residue was partitioned between water and EtOAc. The aqueous phase was dried over MgSO₄ and concentrated to give the title compound (1.37 g, 98%). ¹H NMR (400 MHz, CDCl₃) δ ppm 2.12-2.41 (m, 5H), 2.45-2.62 (m, 2H), 3.64 (d, 1H), 3.75 (d, 1H), 4.03-4.13 (m, 1H), 7.30 (d, 1H), 7.71 (d, 1H). MS m/z 317.4, 319.4 and 321.4 [M+H]⁺.

### Example 3b

### 8-Chloro-4-methyl-2-(2,2,2-trifluoroethyl)-2,3,4,5-tetrahydropyrido[3,2-f][1,4]oxazepine

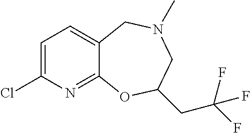

Sodium tert-butoxide (162 mg, 1.68 mmol) was added to a solution of 1-(((2,6-dichloropyridin-3-yl)methyl)(methyl)amino)-4,4,4-trifluorobutan-2-ol (Example 3a, 485 mg, 1.53 mmol) in toluene (5 mL). The resulting mixture was heated to 40° C. and stirred for 16 h. A second addition of sodium tert-butoxide (50 mg, 0.52 mmol) was made and the mixture was stirred another 6 h. The reaction mixture was diluted with EtOAc, washed with water, dried over MgSO₄ and concentrated. The residue was purified by column chromatography using silica stationary phase and gradient elution with increasing concentration of MeOH, from 0 to 7%, in DCM to give a mixture (314 mg) containing the title compound together with a side product and the starting material in approximately 7:2:1 ratio. This mixture was used as such in the following reaction. MS m/z 281.6 and 283.5 [M+H]⁺.

### Example 4

### (+)-N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine; isomer 1

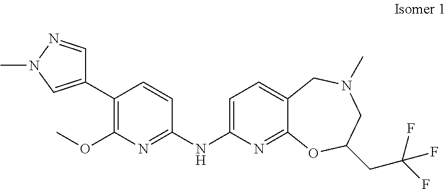

Chiral separation of the isomers of Example 3 (0.200 g, 0.45 mmol) using SFC chromatography [Column: Chiralpak AD-H (21.2*250 mm), Mobile phase: 40% EtOH; 60% CO₂ Flow: 50 ml/min] gave N-(6-methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine, isomer 1 (72 mg, 36%). Isomer 1 has positive optical rotation. ¹H NMR (500 MHz, DMSO-d₆) δ ppm 2.30 (s, 3H), 2.59-2.74 (m, 2H), 2.81 (dd, 1H), 2.90-2.97 (m, 1H), 3.59 (s, 2H), 3.85 (s, 3H), 3.99 (s, 3H), 4.22-4.35 (m, 1H), 7.20 (d, 1H), 7.50-7.63 (m, 2H), 7.79-7.88 (m, 2H), 8.02 (s, 1H), 9.56 (s, 1H). MS m/z 449 [M+H]⁺.

### Example 5

### (−)-N-(6-Methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine; isomer 2

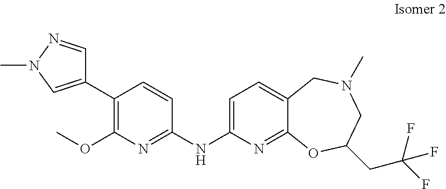

Separation as in Example 4 gave N-(6-methoxy-5-(1-methyl-1H-pyrazol-4-yl)pyridin-2-yl)-4-methyl-2-(2,2,2-trifluoroethyl)-2,3,4,5-tetrahydropyrido[3,2-f][1,4]oxazepin-8-amine, isomer 2 (69 mg, 34%). Isomer 2 has negative optical rotation. ¹H NMR (500 MHz, DMSO-d₆) δ ppm 2.30 (s, 3 H), 2.59-2.74 (m, 2H), 2.81 (dd, 1H), 2.90-2.97 (m, 1H), 3.59 (s, 2H), 3.85 (s, 3H), 3.99 (s, 3 H), 4.25-4.33 (m, 1H), 7.20 (d, 1H), 7.52-7.62 (m, 2H), 7.81-7.86 (m, 2H), 8.02 (s, 1H), 9.56 (s, 1H). MS m/z 449 [M+H]⁺.

### Example 6

### N-(6-Methoxy-5-(4-methyl-1H-imidazol-1-yl)pyridin-2-yl)-4-methyl-2-(2,2,2-trifluoroethyl)-2,3,4,5-tetrahydropyrido[3,2-f][1,4]oxazepin-8-amine

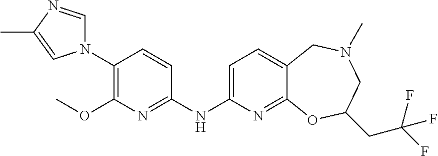

6-Methoxy-5-(4-methyl-1H-imidazol-1-yl)pyridin-2-ylamine (Example 6a, 109 mg, 0.53 mmol), palladium(II)acetate (12.0 mg, 0.05 mmol), 2-(dicyclohexylphosphino)biphenyl (18.7 mg, 0.05 mmol) and cesium carbonate (261 mg, 0.80 mmol) were weighed into a microwave vial. The vial was capped and a solution of crude 8-chloro-4-methyl-2-(2,2,2-trifluoroethyl)-2,3,4,5-tetrahydropyrido[3,2-f][1,4]oxazepine (Example 3b, 150 mg, 0.53 mmol) in DME (4 mL) was added via a syringe. The vial was flushed with argon and the reaction mixture was heated to 100° C. in a microwave apparatus for 2 h. Palladium(II)acetate (12.0 mg, 0.05 mmol) and 2-(dicyclohexylphosphino)biphenyl (18.7 mg, 0.05 mmol) were added and the mixture was heated to 100° C. for 2 h, this was repeated 4 times. The reaction mixture was diluted with DCM, filtered and concentrated. The residue was purified by column chromatography on silica using gradient elution with increasing concentration of MeOH, from 0 to 8%, in DCM to give a material that was further purified by HPLC to give the title compound (8.0 mg, 3.3%). ¹H NMR (500 MHz, CDCl₃) δ ppm 2.30 (s, 3H), 2.39 (m, 1H), 2.45 (s, 3H), 2.71 (m, 1H), 2.91-2.98 (m, 1H), 2.98-3.03 (m, 1H), 3.60 (d, 1H), 3.75 (d, 1H), 3.98 (s, 3H), 4.40-4.48 (m, 1H), 6.87 (s, 1H), 6.98 (d, 1H), 7.28 (s, 1 H), 7.41-7.50 (m, 3H), 7.65 (s, 1H). MS m/z 449.2 [M+H]⁺.

### Example 6a

### 6-Methoxy-5-(4-methylimidazol-1-yl)pyridin-2-ylamine

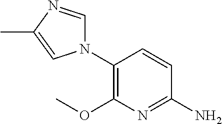

A mixture of 5-bromo-6-methoxypyridin-2-amine (CAS 1211533-83-3, 573 mg, 2.82 mmol), 4-methyl-1H-imidazole (CAS 822-36-6, 324 mg, 3.95 mmol), copper(I)iodide (107 mg, 0.56 mmol) and cesium carbonate (1839 mg, 5.64 mmol) in DMF (5 mL) was heated to 140° C. under argon atmosphere in a microwave reactor for 1 h and then at 150° C. for 1 h. The reaction mixture was diluted with DCM and MeOH and filtered through a plug of Celite. The solvent was evaporated and the residue was purified by HPLC to give the title product (128 mg, 22%). ¹H NMR (500 MHz, DMSO-d₆) δ ppm 2.11 (m, 3H) 3.77 (s, 3H) 6.05 (d, 1H) 6.19 (s, 2H) 6.92 (t, 1H) 7.33 (d, 1H) 7.54 (d, 1H). MS m/z 205.1 [M+H]⁺.

### Example 7

### [6-Methoxy-5-(6-methyl-pyrimidin-4-yl)-pyridin-2-yl]-[6-methyl-8-(2,2,2-trifluoro-ethyl)-5,6,7,8-tetrahydro-9-oxa-1,6-diaza-benzocyclohepten-2-yl]-amine

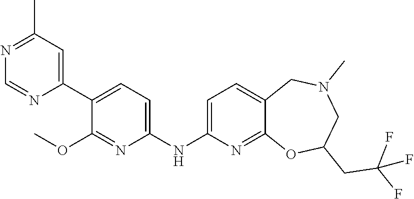

Palladium(II)acetate (33 mg, 0.15 mmol) and Xantphos (173 mg, 0.29 mmol) were added to a degassed mixture of 8-chloro-4-methyl-2-(2,2,2-trifluoroethyl)-2,3,4,5-tetrahydropyrido[3,2-f][1,4]oxazepine (Example 3b, 140 mg, 0.50 mmol), 6-methoxy-5-(6-methyl-pyrimidin-4-yl)-pyridin-2-ylamine (Example 7d, 108 mg, 0.50 mmol) and cesium carbonate (211 mg, 0.64 mmol) in 1,4-dioxane (12 mL). The reaction mixture was purged with nitrogen for 15 min and then heated in a microwave reactor at 145° C. for 1.5 h. The reaction mixture cooled to r.t., diluted with ethyl acetate (60 mL) and filtered. The filtrate was concentrated in vacuo and the residue was purified by flash column chromatography using 5% MeOH in DCM to give the title compound (52 mg, 23%). ¹H NMR (400 MHz, CDCl₃) δ ppm 2.31-2.45 (m, 1H), 2.45 (s, 3H) 2.57 (s, 3H) 2.93-3.01 (m, 1H) 3.61 (d, 1H) 3.76 (d, 1H) 4.11 (s, 3H) 4.42-4.47 (m, 1H) 6.84 (d, 1H) 7.33 (s, 1H) 7.50 (d, 1H) 7.70 (d, 1H) 7.93 (s, 1H) 8.51 (d, 1H) 9.06 (d, 1H). ¹⁹F NMR (376 MHz, CDCl₃) δ ppm −63.82. ESMS m/z 461.1 [M+H]⁺.

### Example 7a

### 5,6-Dibromo-pyridin-2-ylamine

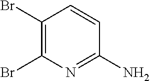

NBS (5.1 g, 28.6 mmol) was added to a solution of 6-bromo-pyridin-2-ylamine (CAS 19798-81-3, 10 g, 57.8 mmol) in MeCN (400 mL) at 0° C. The reaction mixture was stirred for 1 h at 0° C. and then a second portion of NBS (5.1 g, 28.6 mmol) was added. The reaction mixture was allowed to warm to r.t. and stirred o.n. The reaction mixture was concentrated to 20 mL and diluted with water (100 mL). The precipitated solid was collected by filtration, washed with hot water (3×100 mL) and recrystallised from EtOH/Et₂O to give the title compound (12 g, 82%). ¹H NMR (400 MHz, CDCl₃) δ ppm 4.60 (br. s., 2H), 6.34 (d, 1H), 7.54 (d, 1H). ESMS m/z 250.8, 252.8, 254.8 [M+H]⁺.

### Example 7b

### 5-Bromo-6-methoxy-pyridin-2-ylamine

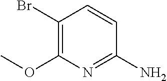

Sodium metal (1.8 g, 78.2 mmol) was added to dry MeOH (70 mL) at −20° C. in small portions. The solution was stirred for 4 h at 0° C. and added to a pressure vessel containing 5,6-dibromo-pyridin-2-ylamine (Example 7a, 10 g, 39.8 mmol). The reaction mixture was heated in a sealed tube at 120° C. for 24 h. The solvent was removed under reduced pressure and the residue was diluted with water. The aqueous phase was neutralized using 2 M HCl and extracted with EtOAc (3×100 mL). The combined extracts were dried over MgSO₄, filtered and concentrated in vacuo. The residue was purified by flash column chromatography using a gradient of 5 to 15% acetone in hexane to afford the title compound (6.6 g, 82%). ¹H NMR (400 MHz, CDCl₃) δ ppm 3.91 (s, 3H), 4.31 (br. s., 2 H), 5.99 (d, 1H), 7.48 (d, 1H). ESMS m/z 202.8, 204.8 [M+H]⁺.

### Example 7c

### 6-Methoxy-5-(4,4,5,5-tetramethyl-[1,3,2]dioxaborolan-2-yl)-pyridin-2-ylamine

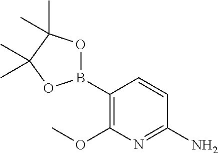

Potassium acetate (303 mg, 3.08 mmol) was added to a mixture of 5-bromo-6-methoxy-pyridin-2-ylamine (Example 7b, 210 mg, 1.03 mmol) and 4,4,5,5,4′,4′,5′,5′-octamethyl-[2,2′]bi[[1,3,2]dioxaborolanyl] (523 mg, 2.05 mmol) in 1,4-dioxane (15 mL). The reaction mixture was purged with nitrogen for 30 min and Pd(dppf)Cl₂ (226 mg, 0.30 mmol) was added. The reaction mixture was heated at 100° C. for 3 h, then cooled to r.t., diluted with ethyl acetate (60 mL) and filtered. The filtrate was concentrated in vacuo and the residue was purified by flash column chromatography (silica gel pre-neutralized with TEA) using 30% EtOAc in hexane to afford the title compound (72 mg, 28%). ¹H NMR (400 MHz, CDCl₃) δ ppm 1.32 (s, 12H) 3.88 (s, 3H) 4.44 (br. s., 2H) 6.03 (d, 1H) 7.78 (d, 1H). ESMS m/z 251.1 [M+H]⁺.

### Example 7d

### 6-Methoxy-5-(6-methyl-pyrimidin-4-yl)-pyridin-2-ylamine

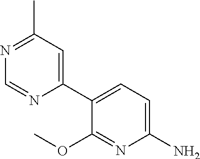

PdCl₂(PPh₃)₂ (20 mg, 0.02 mmol) was added to a degassed mixture of 4-bromo-6-methyl-pyrimidine (74 mg, 0.42 mmol), 6-methoxy-5-(4,4,5,5-tetramethyl-[1,3,2]dioxaborolan-2-yl)-pyridin-2-ylamine (Example 7c, 72 mg, 0.28 mmol) and K₂CO₃ (115 mg, 0.83 mmol) in a mixture of DME:EtOH:water (6:2:1, 14 mL). The reaction mixture was purged with nitrogen for 15 min and then heated in a sealed tube at 100° C. for 1 h. The reaction mixture was cooled to r.t., diluted with ethyl acetate, filtered and concentrated under reduced pressure. The residue was purified by flash column chromatography using 5-10% EtOAc in DCM to afford the title compound (52 mg, 57%). ¹H NMR (400 MHz, CDCl₃) δ ppm 2.54 (s, 3H) 4.00 (s, 3H) 4.62 (br. s., 2H) 6.22 (d, 1H) 7.89 (s, 1H) 8.39 (d, 1H) 9.02 (s, 1H). ESMS m/z 217.1 [M+H]⁺.

### Example 8

### (2-Methoxy-2′-methyl-[3,4′]bipyridinyl-6-yl)-[6-methyl-8-(2,2,2-trifluoro-ethyl)-5,6,7,8-tetrahydro-9-oxa-1,6-diaza-benzocyclohepten-2-yl]-amine

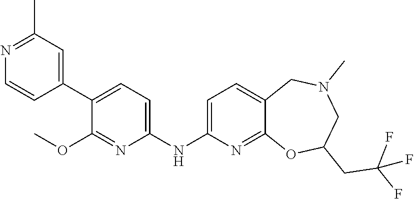

Pd(OAc)₂ (25 mg, 0.11 mmol) and Xantphos (128 mg, 0.22 mmol) were added to a degassed mixture of 8-chloro-4-methyl-2-(2,2,2-trifluoroethyl)-2,3,4,5-tetrahydropyrido[3,2-f][1,4]oxazepine (Example 3b, 105 mg, 0.37 mmol), 2-methoxy-2′-methyl-[3,4]bipyridinyl-6-ylamine (Example 8b, 80 mg, 0.37 mmol) and cesium carbonate (156 mg, 0.47 mmol) in 1,4-dioxane (12 mL). The reaction mixture was purged with nitrogen for 15 min and then heated in a microwave reactor at 145° C. for 1.5 h. The reaction mixture was cooled to r.t., diluted with ethyl acetate (60 mL), filtered and concentrated in vacuo. The residue was purified by flash column chromatography using 5% MeOH in DCM to afford the title compound (70 mg, 41%). ¹H NMR (400 MHz, CDCl₃) δ ppm 2.31-2.44 (m, 1H) 2.45 (s, 3H) 2.59 (s, 3H) 2.64-2.78 (m, 1H) 2.90-3.04 (m, 2H) 3.59 (d, 1 H) 3.75 (d, 1H) 4.00 (s, 3H) 4.40-4.48 (m, 1H) 6.90 (d, 1H) 7.30-7.34 (m, 1H) 7.36 (s, 1H) 7.43 (s, 1H) 7.47 (d, 1H) 7.58 (d, 1H) 7.63 (d, 1H) 8.48 (d, 1H). ¹⁹F NMR (376 MHz, CDCl₃) δ ppm −63.84. ESMS m/z 460.1 [M⁺H]⁺.

### Example 8a

### 2-Methyl-4-tributylstannanyl-pyridine

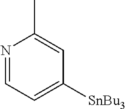

n-BuLi (5.1 mL, 12.75 mmol, 2.5 M in hexane) was added to a solution of 4-bromo-2-methyl-pyridine (2.0 g, 11.6 mmol) in Et₂O (100 mL) at −78° C. The reaction mixture was stirred for 15 min and Bu₃SnCl (3.7 mL, 13.72 mmol) was added. The reaction mixture was stirred at −78° C. for 30 min, then allowed to warm to 0° C. and quenched with saturated NaHCO₃ solution. The mixture was extracted with Et₂O (3×50 mL), and the combined extracts were dried over MgSO₄ and concentrated in vacuo. The residue was purified by flash column chromatography (silica gel pre-neutralized neutralized with TEA) using 5% EtOAc in hexane to afford the title compound (2.1 g, 47%). H NMR (400 MHz, CDCl₃) δ ppm 0.88 (t, 9H) 0.97-1.15 (m, 6H) 1.24-1.37 (m, 6H) 1.42-1.60 (m, 6H) 2.51 (s, 3H) 7.14 (d, 1H) 7.21 (s, 1H) 8.36 (d, 1H).

### Example 8b

### 2-Methoxy-2′-methyl-[3,4′]bipyridinyl-6-ylamine

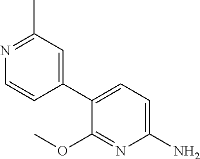

Pd(PPh₃)₄ (682 mg, 0.59 mmol) was added to a degassed mixture of 5-bromo-6-methoxy-pyridin-2-ylamine (Example 7b, 1.2 g, 5.91 mmol) and 2-methyl-4-tributylstannanyl-pyridine (Example 8a, 2.7 g, 8.12 mmol) in xylene (200 mL). The reaction mixture was purged with nitrogen for 15 min and then heated in a sealed tube at 145° C. o.n. The mixture was cooled to r.t. and concentrated under reduced pressure. The residue was purified by flash column chromatography using 50% EtOAc in DCM to obtain the title compound (950 mg, 75%). ¹H NMR (400 MHz, CDCl₃) δ ppm 2.57 (s, 3 H) 3.92 (s, 3H) 4.47 (br. s., 2H) 6.17 (d, 1H) 7.29 (d, 1H) 7.33 (s, 1H) 7.50 (d, 1H) 8.45 (d, 1H). ESMS m/z 216.0 [M+H]⁺.

### Example 9

### N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine

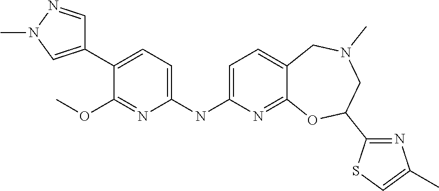

To 8-chloro-4-methyl-2-(4-methylthiazol-2-yl)-2,3,4,5-tetrahydropyrido[3,2-f][1,4]oxazepine (Example 9e, 207 mg, 0.70 mmol) in DME (3 mL) were 6-methoxy-5-(1-methyl-1H-pyrazol-4-yl)pyridin-2-ylamine (Example 2a, 143 mg, 0.70 mmol), cesium carbonate (342 mg, 1.05 mmol), 2-(dicyclohexylphosphino)biphenyl (24.5 mg, 0.07 mmol) and palladium acetate (15.7 mg, 0.07 mmol) added. The reaction was heated to 110° C. for 60 min under N₂ atmosphere. The solids were filtered off and washed with DCM, the solvents were evaporated and the crude product was purified by silica flash chromatography using DCM:[DCM:MeOH:NH₃=90:10:1]=100:0 to 35:65 as gradient to give the title compound (183 mg, 56%). ¹H NMR (400 MHz, CDCl₃) δ ppm 2.46 (d, 3 H) 2.50 (s, 3H) 3.17 (dd, 1H) 3.56-3.68 (m, 2H) 3.91-3.98 (m, 4H) 4.06 (s, 3H) 5.36 (dd, 1H) 6.69 (d, 1H) 6.92 (d, 1H) 7.29 (s, 1H) 7.51 (d, 1H) 7.69 (d, 1H) 7.72 (d, 1H) 7.78 (s, 1H) 7.81 (s, 1H). MS m/z 464.5 [M+H]⁺.

### Example 9a

### (4-Methyl-thiazol-2-yl)-2-nitro-ethanol

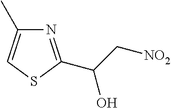

A mixture of potassium carbonate (25.6 g, 0.19 mol) and nitromethane (59 mL, 1.10 mol) in EtOH (120 mL) was stirred for 5 min at −10° C. Neat 4-methyl-2-thiazole carboxaldehyde (CAS 13750-68-0, 11.8 g, 0.09 mol) was added and the temperature of the reaction was maintained between −10° C. and −5° C. for 1 h. The resulting suspension was filtered and crushed ice (100 mL) was added to the filtrate. The aqueous layer was then extracted with EtOAc (3×100 mL). The combined organic extracts were washed with brine (100 mL), dried over sodium sulfate and filtered. The filtrate was concentrated under reduced pressure to afford crude 1-(4-methyl-thiazol-2-yl)-2-nitro-ethanol (15.0 g, 86%) which was used in the next step without further purification. ¹H NMR (400 MHz, CDCl₃) δ ppm 2.43 (s, 3H) 4.00 (br. s., 1H) 4.74 (dd, 1H) 4.98 (dd, 1H) 5.68 (d, 1H) 6.93 (s, 1H). MS m/z 189 [M+H]⁺.

### Example 9b

### 2-Amino-1-(4-methylthiazol-2-yl)ethanol

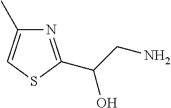

A mixture of (4-methyl-thiazol-2-yl)-2-nitro-ethanol (Example 9a, 15.0 g, 0.08 mol) and 10% palladium on carbon (1.5 g, 10 wt. %) in dry MeOH (200 mL) was shaken in a Parr apparatus under a hydrogen atmosphere (50 psi) for 24 h. The reaction mixture was filtered through a pad of Celite and a fresh batch of catalyst (10% Pd/C, 1.5 g) was added to the filtrate and the resulting mixture was shaken under a hydrogen atmosphere (50 psi) for 24 h. This procedure was repeated one more time (total reaction time: 72 h) and the filtrate was concentrated under reduced pressure. The residue was purified by flash column chromatography using a gradient of 5% to 10% MeOH in DCM to afford the title compound (4.30 g, 34%). ¹H NMR (400 MHz, DMSO-d₆) δ ppm 1.55 (br. s., 2H) 2.30 (s, 3H) 2.66 (dd, 1H) 2.87 (dd, 1H) 4.62 (dd, 1H) 6.06 (br. s., 1H) 7.09 (s, 1H). MS m/z 159 [M+H]⁺.

### Example 9c

### 2-((2,6-Dichloropyridin-3-yl)methylamino)-1-(4-methylthiazol-2-yl)ethanol

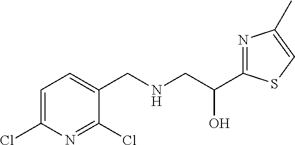

2,6-Dichloronicotinaldehyde (CAS 55304-73-9, 2.78 g, 15.80 mmol), 2-amino-1-(4-methylthiazol-2-yl)ethanol (Example 9b, 2.5 g, 15.80 mmol) and acetic acid (0.45 mL, 7.90 mmol) in MeOH (50 mL) were stirred at 0° C. for 5 min under N₂-atmosphere and then allowed to warm up to r.t. and stirred for 40 min. Sodium cyanoborohydride (1.489 g, 23.70 mmol) was added in portions, the inert atmosphere was restored and the mixture was stirred at r.t. for 2 h. MeOH (containing 1% NH₃) was added to adjust the pH to 7 and then the solvent was removed in vacuo. The crude product was purified by silica flash chromatography using DCM:[DCM:MeOH:NH₃=90:10:1]=100:0 to 40:60 as gradient to give the title compound (4.2 g, 84%). ¹H NMR (400 MHz, CDCl₃) δ ppm 2.42 (d, 3 H) 3.09-3.21 (m, 2H) 3.65-3.81 (m, 2H) 3.95 (s, 2H) 5.06 (t, 1H) 6.86 (d, 1H) 7.28 (d, 1H) 7.76 (d, 1H). MS m/z 318.5 and 320.5 [M+H]⁺.

### Example 9d

### 8-Chloro-2-(4-methylthiazol-2-yl)-2,3,4,5-tetrahydropyrido[3,2-f][1,4]oxazepine

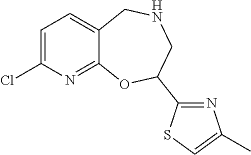

Sodium tert-butoxide (2.01 g, 20.9 mmol) was added in portions to a stirred solution of 2-((2,6-dichloropyridin-3-yl)methylamino)-1-(4-methylthiazol-2-yl)ethanol (Example 9c, 3.91 g, 12.3 mmol) in THF (45 mL) at 0° C. The mixture was set under N₂-atmosphere, stirred for 5 min at 0° C. and was then allowed to warm-up to r.t. and stirred o.n. More sodium tert-butoxide (200 mg, 2 mmol) was added and the mixture was allowed to stir for another hour. Water was added to the reaction mixture and the phases were separated. The aqueous layer was extracted with EtOAc. The organic layer was dried over anhydrous sodium sulfate and concentrated. The residue was purified on a 120 g silica column using DCM:(DCM:MeOH:NH₃=90:10:1)=100:0 to 50:50 gradient. Some fractions were repurified by the same chromatographic method to give the title compound (1.30 g, 37.4%). MS m/z 282.6 [M+H]⁺.

### Example 9e

### 8-Chloro-4-methyl-2-(4-methylthiazol-2-yl)-2,3,4,5-tetrahydropyrido[3,2-f][1,4]oxazepine

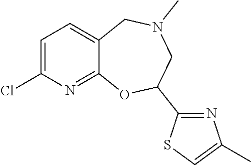

Formaldehyde 37% (3.42 mL, 46.0 mmol) and acetic acid (0.132 mL, 2.30 mmol) were added to a solution of 8-chloro-2-(4-methylthiazol-2-yl)-2,3,4,5-tetrahydropyrido[3,2-f][1,4]oxazepine (Example 9d, 1.296 g, 4.60 mmol) in MeOH (15 mL) at r.t. under N₂-atmosphere. The mixture was stirred for 15 min and then sodium cyanoborohydride (0.434 g, 6.90 mmol) was added and the reaction stirred for 20 h at r.t. MeOH (containing 1% NH₃) was added to adjust the pH to 7 and then the solvent was removed in vacuo. Sat. NaHCO₃-solution and EtOAc were added to the residue and the phases were separated. The aqueous layer was dried over anhydrous sodium sulfate, filtered and concentrated. The crude product was purified by silica flash chromatography using DCM:[DCM:MeOH:NH₃=90:10:1]=100:0 to 50:50 as gradient to give the title compound (768 mg, 56%). ¹H NMR (400 MHz, CDCl₃) δ ppm 2.46 (d, 3H) 2.51 (s, 3H) 3.20 (dd, 1H) 3.62 (d, 1 H) 3.72 (d, 1H) 3.96 (d, 1H) 5.37 (dd, 1H) 6.93 (d, 1H) 7.13 (d, 1H) 7.55 (d, 1H). MS m/z 296.5 [M+H]⁺.

### Example 10

### (+)-N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine; isomer 1

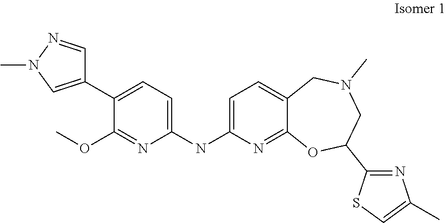

The two isomers of Example 9 (140 mg, 0.30 mmol) were separated using SFC chromatography [Column: Chiralcel OJ-H, 4.6*250 mm; 5 μm; Mobile phase: 20% MeOH+0.1% DEA; 80% CO₂; Flow: 50 mL/min] yielding N-(6-methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine, isomer 1 (53 mg, 38%) which has a positive optical rotation. ¹H NMR (500 MHz, DMSO-d₆) δ ppm 2.37-2.40 (m, 6 H) 2.98-3.06 (m, 1H) 3.44-3.51 (m, 1H) 3.63-3.81 (m, 2H) 3.86 (s, 3H) 4.01 (s, 3H) 5.33 (s, 1 H) 7.00 (d, 1H) 7.31 (d, 1H) 7.65 (d, 1H) 7.80-7.87 (m, 3H) 8.02 (s, 1H) 9.74 (s, 1H). MS m/z 464.2 [M+H]⁺.

### Example 11

### (−)-N-(6-Methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine; isomer 2

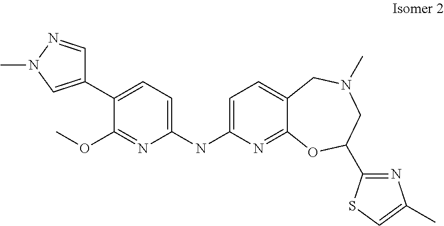

Chiral separation of Example 9 as in Example 10 yielded N-(6-methoxy-5-(1-methyl-1H-pyrazol-4-yl)pyridin-2-yl)-4-methyl-2-(4-methylthiazol-2-yl)-2,3,4,5-tetrahydropyrido[3,2-f][1,4]oxazepin-8-amine, isomer 2 (48.0 mg, 34%, the second to elute) which has a negative optical rotation. ¹H NMR (500 MHz, DMSO-d₆) δ ppm 2.35-2.41 (m, 6H) 3.01 (dd, 1H) 3.47 (d, 1H) 3.64-3.81 (m, 2H) 3.86 (s, 3H) 4.01 (s, 3H) 5.34 (dd, 1H) 7.00 (d, 1H) 7.31 (d, 1H) 7.65 (d, 1H) 7.80-7.87 (m, 3H) 8.02 (s, 1H) 9.74 (s, 1H). MS m/z 464.2 [M+H]⁺.

### Example 12

### N-(6-Methoxy-5-(4-methyl-1H-imidazol-1-yl)pyridin-2-yl)-4-methyl-2-(4-methylthiazol-2-yl)-2,3,4,5-tetrahydropyrido[3,2-f][1,4]oxazepin-8-amine

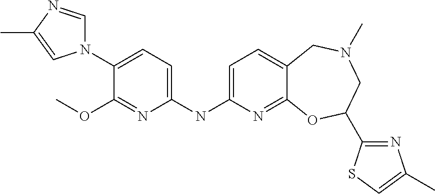

Preparation in analogy with Example 9, using 8-chloro-4-methyl-2-(4-methylthiazol-2-yl)-2,3,4,5-tetrahydropyrido[3,2-f][1,4]oxazepine (Example 9e, 50 mg, 0.17 mmol) and 6-methoxy-5-(4-methyl-1H-imidazol-1-yl)pyridin-2-ylamine (Example 6a, 34.5 mg, 0.17 mmol) as starting materials giving the title compound (16 mg, 20%). ¹H NMR (500 MHz, DMSO-d₆) δ ppm 2.14 (s, 3H) 2.37 (s, 3H) 2.38 (s, 3H) 2.99-3.06 (m, 1H) 3.46 (s, 1H) 3.66-3.81 (m, 2H) 3.95 (s, 3H) 5.33-5.37 (m, 1H) 7.04 (d, 1H) 7.07 (s, 1H) 7.30 (s, 1H) 7.64-7.71 (m, 3H) 7.79 (d, 1H) 9.96 (s, 1H). MS m/z 464.2 [M+H]⁺.

### Example 13

### 2-Cyclopropyl-N-(6-methoxy-5-(1-methyl-1H-pyrazol-4-yl)pyridin-2-yl)-4,6-dimethyl-2,3,4,5-tetrahydropyrido[3,2-f][1,4]oxazepin-8-amine

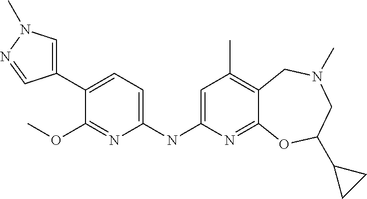

A mixture of 8-chloro-2-cyclopropyl-4,6-dimethyl-2,3,4,5-tetrahydropyrido[3,2-f][1,4]oxazepine (Example 13c, 0.2 g, 0.79 mmol), 6-methoxy-5-(1-methyl-1H-pyrazol-4-yl)pyridin-2-ylamine (Example 2a, 0.16 g, 0.79 mmol), acetoxy(2′-(di-tert-butylphosphino)biphenyl-2-yl)palladium (0.018 g, 0.04 mmol) and cesium carbonate (0.387 g, 1.19 mmol) in DME (3 mL) was heated in a microwave at 100° C. for 1 h. The reaction mixture was filtered through Celite and rinsed with DCM. The solvent was evaporated and the crude product was purified by silica flash chromatography using a gradient of MeOH (0 to 5%) in DCM. The fractions containing product were collected and purified by preparative chromatography giving the title compound (14 mg, 4.2%). ¹H NMR (500 MHz, DMSO-d₆) δ ppm 0.31-0.39 (m, 1H), 0.42 (td, 1H), 0.46-0.58 (m, 2H), 0.96-1.07 (m, 1 H), 2.27 (s, 3H), 2.30 (s, 3H), 2.80-2.88 (m, 1H), 2.89-2.96 (m, 1H), 3.34-3.39 (m, 1H), 3.45 (d, 1H), 3.72 (d, 1H), 3.85 (s, 3H), 4.01 (s, 3H), 6.93 (d, 1H), 7.68 (s, 1H), 7.81-7.85 (m, 2H), 8.01 (s, 1H), 9.54 (s, 1H). MS m/z 421.5 [M+H]⁺.

### Example 13a

### 1-Cyclopropyl-2-((2,6-dichloro-4-methylpyridin-3-yl)methylamino)ethanol

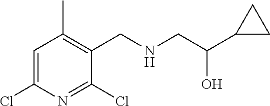

Potassium carbonate (5.65 g, 40.88 mmol) was added to a stirred solution of 3-(bromomethyl)-2,6-dichloro-4-methylpyridine (Example 1b, 4.169 g, 16.35 mmol) and 2-amino-1-cyclopropylethanol (CAS 54120-02-4, 2.15 g, 21.26 mmol) in MeCN (35 mL) at r.t. The reaction mixture was stirred for 4 h. The reaction mixture was filtered and the filtrate was concentrated. The crude product was purified by silica flash chromatography using a gradient of MeOH (0 to 4%) in DCM giving the title compound (2.66 g, 59%). ¹H NMR (500 MHz, DMSO-d₆) δ ppm 0.13-0.18 (m, 1H), 0.20-0.25 (m, 1H), 0.30-0.35 (m, 2H), 0.73-0.82 (m, 1H), 2.44 (s, 3H), 2.54 (dd, 1H), 2.62 (dd, 1H), 2.92-2.98 (m, 1H), 3.79-3.83 (m, 2H), 4.52 (d, 1H), 7.46 (s, 1H). MS m/z 275.0, 277.0 and 278.9 [M+H]⁺.

### Example 13b

### 8-Chloro-2-cyclopropyl-6-methyl-2,3,4,5-tetrahydropyrido[3,2-f][1,4]oxazepine

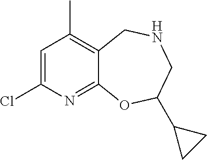

Sodium tert-butoxide (1.32 g, 13.7 mmol) was added in two portions to a stirred solution of 1-cyclopropyl-2-((2,6-dichloro-4-methylpyridin-3-yl)methylamino)ethanol (Example 13a, 2.51 g, 9.13 mmol) in THF (20 mL) at 0° C. After 10 min the ice-water bath was removed and the reaction mixture was stirred at r.t. o.n. Another 0.5 eq sodium tert-butoxide was added and the reaction stirred for 6 h, still another 0.5 eq sodium tert-butoxide was added and the reaction stirred at r.t. for 3 days. MeOH was added to quench the reaction and the solvent was evaporated. The residue was partitioned between sat. NaHCO₃ (aq) and DCM. The organic layer was separated and the aqueous layer was extracted with DCM. The combined organic layer was dried over NaSO₄ and concentrated to give crude 8-chloro-2-cyclopropyl-6-methyl-2,3,4,5-tetrahydropyrido[3,2-f][1,4]oxazepine (2.35 g) which was used in the next step without further purification. MS m/z 239.0 [M+H]⁺.

### Example 13c

### 8-Chloro-2-cyclopropyl-4,6-dimethyl-2,3,4,5-tetrahydropyrido[3,2-f][1,4]oxazepine

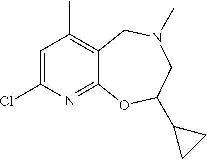

Formaldehyde (2.73 mL, 98.6 mmol) and acetic acid (0.565 mL, 9.86 mmol) were added to a stirred solution of 8-chloro-2-cyclopropyl-6-methyl-2,3,4,5-tetrahydropyrido[3,2-f][1,4]oxazepine (Example 13b, 2.35 g, 9.86 mmol) in MeOH (15 mL) at 0° C. The reaction was stirred at r.t. for 15 min. The reaction mixture was cooled to 0° C. and sodium cyanoborohydride (0.651 g, 10.35 mmol) was added. After 5 min the reaction mixture was stirred at r.t. for 2 h before being quenched with a small amount of MeOH. The solvent was evaporated and the residue was partitioned between sat. NaHCO₃ (aq) and DCM. The aqueous layer was extracted with DCM. The combined organic layer was dried over Na₂SO₄ and concentrated. The crude product was purified by silica flash chromatography using a gradient of MeOH (0 to 5%) in DCM giving the title compound (1.30 g, 52%). ¹H NMR (500 MHz, DMSO-d₆) δ ppm 0.29-0.37 (m, 1H), 0.37-0.44 (m, 1H), 0.46-0.59 (m, 2H), 0.96-1.07 (m, 1H), 2.28 (s, 3H), 2.31 (s, 3H), 2.79-3.00 (m, 2H), 3.39-3.48 (m, 1H), 3.50 (d, 1H), 3.76 (d, 1H), 7.08 (s, 1H). MS m/z 253.0 [M+H]⁺.

### Example 14

### (R)-4-Methyl-N-(5-(4-methyl-1H-imidazol-1-yl)pyridin-2-yl)-2-phenyl-2,3,4,5-tetrahydropyrido[3,2-f][1,4]oxazepin-8-amine

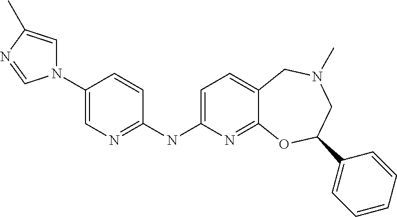

(R)-8-Chloro-4-methyl-2-phenyl-2,3,4,5-tetrahydropyrido[3,2-f][1,4]oxazepine (Example 14c, 221 mg, 0.80 mmol), 5-(4-methyl-1H-imidazol-1-yl)pyridin-2-ylamine (Example 1f, 140 mg, 0.80 mmol), cesium carbonate (393 mg, 1.21 mmol) and acetoxy(2′-(di-tert-butylphosphino)biphenyl-2-yl)palladium (18.6 mg, 0.04 mmol) were added to a microwave vial. DME (2.5 mL) and EtOH (0.250 mL) were added. The reaction mixture was flushed with argon and the mixture was run in the microwave oven for 90 min at 100° C. The solids were filtered off and washed with DCM. The solvent was removed and the crude product was purified using flash chromatography, 0-10% MeOH in DCM yielding the title product (76 mg, 23%). ¹H NMR (500 MHz, DMSO-d₆) δ ppm 2.15 (s, 3 H) 2.36 (s, 3H) 2.99-3.01 (m, 1H) 3.60 (m, 1H) 3.72 (d, 2H) 5.10 (dd, 1H) 7.31-7.36 (m, 2H) 7.40 (t, 2H) 7.46-7.49 (m, 2H) 7.55-7.58 (m, 2H) 7.61-7.64 (m, 1H) 7.87 (dd, 1H) 8.01 (d, 1 H) 8.46 (d, 1H) 9.91 (s, 1H). MS m/z 413.2 [M+H]⁺.

### Example 14a

### (R)-2-((2,6-Dichloropyridin-3-yl)methylamino)-1-phenylethanol

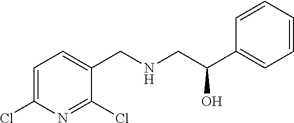

N-Ethyldiisopropylamine (757 μl, 4.34 mmol) was added to a solution of 2,6-dichloro-3-(chloromethyl)pyridine (CAS 41789-37-1, 568 mg, 2.89 mmol) and (R)-2-amino-1-phenylethanol (CAS 2549-14-6, 436 mg, 3.18 mmol) in DMF and the solution was stirred at r.t. for 16 h. The solvent was evaporated at reduced pressure, the residue was dissolved in DCM and the solution was washed with dilute aqueous HCl, water and brine, dried over Na₂SO₄ and evaporated. The compound was crystallised from diethyl ether to give the title product (568 mg, 66%). ¹H NMR (400 MHz, DMSO-d₆) δ ppm 2.64 (d, 2H) 3.79 (s, 2H) 4.66 (m, 1H) 5.35 (d, 1H) 7.23 (m, 1H) 7.31 (m, 4H) 7.55 (d, 1H) 7.96 (d, 1H).

### Example 14b

### (R)-2-(((2,6-Dichloropyridin-3-yl)methyl)(methyl)amino)-1-phenylethanol

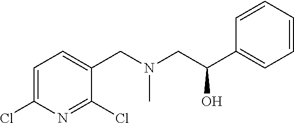

To a solution of (R)-2-((2,6-dichloropyridin-3-yl)methylamino)-1-phenylethanol (Example 14a, 565 mg, 1.9 mmol) in THF (8 mL) was added formaldehyde (0.177 mL, 2.38 mmol) and acetic acid (0.027 mL, 0.48 mmol). The resulting mixture was stirred for 40 min prior to the addition of sodium cyanoborohydride (209 mg, 3.33 mmol). Stirring was continued for another 40 min and then the reaction was quenched by the addition of water. The mixture was extracted with EtOAc, the combined organic extracts were washed with water and brine, dried over MgSO₄ and evaporated to give the title product (598 mg, 100%). ¹H NMR (400 MHz, DMSO-d₆) δ ppm 2.28 (s, 3H) 2.53 (m, 1H) 2.61 (dd, 1H) 3.63 (s, 2H) 4.73 (m, 1H) 5.16 (d, 1H) 7.25 (m, 5H) 7.48 (d, 1H) 7.84 (d, 1H). MS m/z 311, 313 [M+H]⁺.

### Example 14c

### (R)-8-Chloro-4-methyl-2-phenyl-2,3,4,5-tetrahydropyrido[3,2-f][1,4]oxazepine

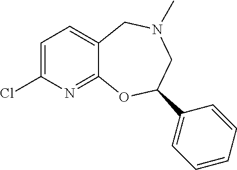

### Method 1:

A 60% dispersion of sodium hydride (84 mg, 2.10 mmol) in mineral oil was washed with hexane under argon atmosphere and residual hexane was evaporated at reduced pressure. The dry sodium hydride was suspended in THF (2 mL) under an argon atmosphere, the suspension was cooled in an ice-water bath and a solution of (R)-2-(((2,6-dichloropyridin-3-yl)methyl)(methyl)amino)-1-phenylethanol (Example 14b, 594 mg, 1.91 mmol) in THF (8 mL) was added via a syringe. The resulting mixture was stirred for 5 min with ice-water bath cooling and then the temperature was raised to r.t. and the stirring was continued for 24 h. The reaction mixture was diluted with EtOAc, washed with water, dried over MgSO₄ and evaporated to give the title product (518 mg, 16%). MS m/z 275, 277 [M+H]⁺.

### Method 2:

Acetic acid (0.069 mL, 1.20 mmol) was added to a solution of (R)-8-chloro-2-phenyl-2,3,4,5-tetrahydropyrido[3,2-f][1,4]oxazepine (Example 14d, 314 mg, 1.20 mmol) and formaldehyde (37% aqueous, 103 mg, 1.26 mmol) in MeOH (5 mL). The solution was stirred at r.t. for 30 min and then sodium cyanoborohydride (114 mg, 1.81 mmol) was added. The resulting mixture was stirred for 1 h. The reaction was quenched by the addition of sat. aqueous NaHCO₃, the MeOH was evaporated at reduced pressure and the aqueous residue was extracted with DCM. The combined organic layers were washed with water, dried over Na₂SO₄ and evaporated to give the title product (308 mg, 93%). ¹H NMR (400 MHz, DMSO-d₆) δ ppm 2.36 (s, 3H) 2.96-3.03 (m, 1H) 3.03-3.12 (m, 1H) 3.75-3.82 (m, 1H) 3.83-3.90 (m, 1H) 5.19 (dd, 1H) 7.26 (d, 1H) 7.34-7.38 (m, 1H) 7.38-7.44 (m, 2H) 7.44-7.49 (m, 2H) 7.82 (d, 1H). MS m/z 275, 277 [M+H]⁺.

### Example 14d

### (R)-8-Chloro-2-phenyl-2,3,4,5-tetrahydropyrido[3,2-f][1,4]oxazepine

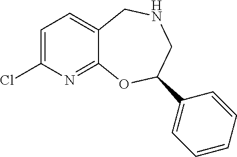

Sodium hydride (60% in mineral oil, 0.306 g, 12.76 mmol) was added in portions to a stirred solution of (R)-2-((2,6-dichloropyridin-3-yl)methylamino)-1-phenylethanol (Example 14a, 2.528 g, 8.51 mmol) in THF (10 mL) at 0° C. After 5 min the reaction mixture was stirred at r.t. for 2 h and then heated to 50° C. for 1 h. The reaction was stirred at r.t. o.n., 0.3 eq sodium hydride (60% in mineral oil) was added and the reaction was stirred at r.t. over the weekend. The solvent was evaporated and the residue was partitioned between DCM and sat. NaHCO₃ (aq). The water layer was extracted twice with DCM. The combined organic phase was dried over MgSO₄ and concentrated. The crude product was purified by silica flash chromatography using a gradient of MeOH (0 to 5%) in DCM giving the title compound (1.716 g, 77%). ¹H NMR (600 MHz, DMSO-d₆) δ ppm 3.07 (dd, 1H) 3.20 (dd, 1H) 3.79 (d, 1H) 4.03 (d, 1H) 5.02 (dd, 1H) 7.21 (d, 1H) 7.34 (t, 1H) 7.38-7.47 (m, 4H) 7.75 (d, 1H). MS m/z 260 [M+H]⁺.

### Example 15

### (R)-4-Methyl-N-(5-(1-methyl-1H-pyrazol-4-yl)pyridin-2-yl)-2-phenyl-2,3,4,5-tetrahydropyrido[3,2-f][1,4]oxazepin-8-amine

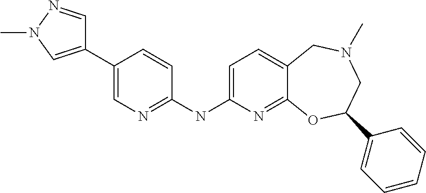

5-(1-Methyl-1H-pyrazol-4-yl)pyridin-2-ylamine (Example 15a, 119 mg, 0.68 mmol), acetoxy(2′-(di-tert-butylphosphino)biphenyl-2-yl)palladium (14.32 mg, 0.03 mmol) and cesium carbonate (302 mg, 0.93 mmol) were charged in a microwave vial, the vial was capped and flushed with argon. A solution of (R)-8-chloro-4-methyl-2-phenyl-2,3,4,5-tetrahydropyrido[3,2-f][1,4]oxazepine (Example 14c, 170 mg, 0.62 mmol) in DME (4.5 mL) was added via a syringe followed by EtOH (0.5 mL). The mixture was heated to 100° C. in a microwave apparatus for 1 h. The reaction mixture was filtered and concentrated. The residue was purified by column chromatography on silica using gradient elution with increasing concentration of MeOH, from 0 to 8%, in DCM to give the title compound (76 mg, 30%). ¹H NMR (500 MHz, DMSO-d₆) δ ppm 2.36 (s, 3H) 2.93-3.05 (m, 2H) 3.71 (m, 2H) 3.85 (s, 3H) 5.09 (dd, 1H) 7.31-7.36 (m, 1H) 7.38-7.45 (m, 3H) 7.46-7.50 (m, 2 H) 7.60 (s, 2H) 7.80 (dd, 1H) 7.81-7.83 (m, 1H) 8.07 (s, 1H) 8.44 (d, 1H) 9.71 (s, 1H). MS m/z 413.1 [M+H]⁺and 411.1 [M−H]⁻.

### Example 15a

### 5-(1-Methyl-1H-pyrazol-4-yl)-pyridin-2-ylamine

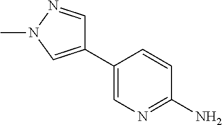

A mixture of 2-amino-5-bromopyridine (CAS 1072-97-5, 3.0 g, 17.3 mmol), 1-methyl-4-(4,4,5,5-tetramethyl-1,3,2-dioxaborolan-2yl)-1H-pyrazole (CAS 761446-44-0, 5.4 g, 26.0 mmol) and potassium carbonate (3.6 g, 26.0 mmol) in DME (60 mL) and water (10 mL) was degassed for 15 min using nitrogen. [1,1′-Bis(di-tert-butylphosphino)-ferrocene]palladium(II) dichloride (562 mg, 0.87 mmol) was added and the reaction mixture was heated in a sealed tube at 90° C. o.n. The mixture was cooled to r.t. and partitioned between EtOAc and aqueous sodium bicarbonate solution. The organic phase was separated and the aqueous layer was re-extracted with EtOAc. The combined extracts were dried over magnesium sulfate, filtered and concentrated under reduced pressure. The residue was purified by flash column chromatography using a gradient of 2 to 5% MeOH in DCM. The desired fractions were collected and concentrated in vacuo, and the solid obtained was washed with diethyl ether to afford the title compound (2.0 g, 66%). ¹H NMR (400 MHz, CDCl₃) δ ppm 3.94 (s, 3H) 4.39 (br. s., 2H) 6.53 (d, 1H) 7.50 (m, 2H) 7.66 (s, 1H) 8.21 (d, 1H). MS m/z 175.1 [M+H]⁺.

### Example 16

### N-(6-Methoxy-5-(1-methyl-1H-pyrazol-4-yl)pyridin-2-yl)-4-methyl-2-(3-methylpyridin-2-yl)-2,3,4,5-tetrahydropyrido[3,2-f][1,4]oxazepin-8-amine

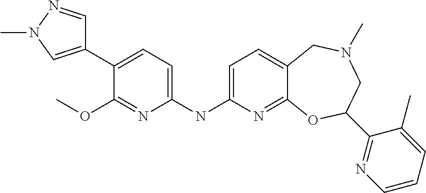

6-Methoxy-5-(1-methyl-1H-pyrazol-4-yl)pyridin-2-ylamine (Example 2a, 0.093 g, 0.46 mmol), cesium carbonate (0.223 g, 0.68 mmol) and acetoxy(2′-(di-tert-butylphosphino)biphenyl-2-yl)palladium (10.54 mg, 0.02 mmol) were added to a microwave vial. 8-Chloro-4-methyl-2-(3-methylpyridin-2-yl)-2,3,4,5-tetrahydropyrido[3,2-f][1,4]oxazepine (Example 16d, 0.132 g, 0.46 mmol) in DME (2 mL) was added. The reaction was set under N₂-atmosphere and heated in a microwave reactor at 110° C. for 1 h. Additional catalyst, [acetoxy(2′-(di-tert-butylphosphino)biphenyl-2-yl)palladium (10.54 mg, 0.02 mmol)], was added and the reaction was heated again in a microwave oven to 110° C. for 1 h. Additional acetoxy(2′-(di-tert-butylphosphino)biphenyl-2-yl)palladium (10.54 mg, 0.02 mmol) was added and the mixture was heated to 110° C. for 3 h in a microwave oven. Additional acetoxy(2′-(di-tert-butylphosphino)biphenyl-2-yl)palladium (10.54 mg, 0.02 mmol) and cesium carbonate (50 mg) was added and the reaction was heated for 2 h at 110° C. in a microwave oven. The solids were filtered off and washed with DME. The solvent was evaporated and the crude product was purified by preparative HPLC. The product was further purified by column chromatography using DCM:[DCM:MeOH:NH₃=90:10:1]=100:0 to 20:80 as gradient to give N-(6-methoxy-5-(1-methyl-1H-pyrazol-4-yl)pyridin-2-yl)-4-methyl-2-(3-methylpyridin-2-yl)-2,3,4,5-tetrahydropyrido[3,2-f][1,4]oxazepin-8-amine (23 mg, 11%). ¹H NMR (400 MHz, CDCl₃) δ ppm 2.47 (s, 3H) 2.55 (s, 3 H) 3.23 (d, 1H) 3.58 (dd, 1H) 3.67 (d, 1H) 3.90-3.98 (m, 4H) 4.07 (s, 3H) 5.33 (d, 1H) 6.67 (d, 1H) 7.15-7.21 (m, 2H) 7.50-7.57 (m, 2H) 7.64-7.72 (m, 2H) 7.77-7.83 (m, 2H) 8.51 (dd, 1 H). MS m/z 458.6 [M+H]⁺.

### Example 16a

### 2-Bromo-1-(3-methylpyridin-2-yl)ethanol

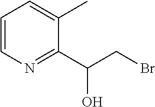

To 2-bromo-1-(3-methylpyridin-2-yl)ethanone (CAS 220270-42-8, 3.5 g, 16.3 mmol) in MeOH (2 mL), at 0° C., sodium borohydride (1.113 g, 29.43 mmol) was slowly added. After the gas evolution had stopped the reaction was allowed to retain r.t. and stirred for 1 h. The pH was adjusted to 7 by 2 M HCl solution. The MeOH was removed in vacuo, the residual liquid was diluted with water and extracted by EtOAc. The organic layer was dried over anhydrous sodium sulfate, filtered and concentrated affording the product (52 mg, 51%). ¹H NMR (400 MHz, CDCl₃) δ ppm 2.38 (s, 3H) 3.55 (dd, 1H) 3.68 (dd, 1H) 4.89 (br. s., 1H) 5.11 (dd, 1H) 7.21 (dd, 1H) 7.51-7.55 (m, 1H) 8.44 (d, 1H). MS m/z 216.4 [M+H]⁺.

### Example 16b

### 2-(Methylamino)-1-(3-methylpyridin-2-yl)ethanol

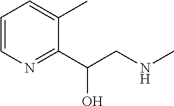

To 2-bromo-1-(3-methylpyridin-2-yl)ethanol (Example 16a, 2.1 g, 9.72 mmol) methanamine (2 M in MeOH, 19.4 mL, 38.9 mmol) and MeOH (14 mL) were added. The mixture was equally divided between two 20 mL microwave vials. The reactions were heated to 100° C. for 5 min. The mixtures were combined, the solvent was removed in vacuo and the residue was purified on silica column using DCM:MeOH (1% NH₃)=100:0 to 50:50 as gradient to give the title compound (1.61 g, quantitative). ¹H NMR (500 MHz, CDCl₃) δ ppm 2.48 (s, 3H) 2.89 (s, 3H) 3.27 (dd, 1H) 3.45-3.51 (m, 1H) 5.65 (dd, 1H) 7.24 (dd, 1H) 7.57 (d, 1H) 8.40 (d, 1H). MS m/z 167 [M+H]⁺.

### Example 16c

### 2-(((2,6Dichloropyridin-3-yl)methyl)(methyl)amino)-1-(3-methylpyridin-2-yl)ethanol

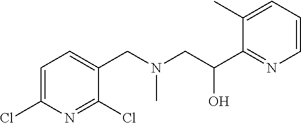

A solution of 2,6-dichloronicotinaldehyde (CAS 55304-73-9, 1.71 g, 9.69 mmol), 2-(methylamino)-1-(3-methylpyridin-2-yl)ethanol (Example 16b, 1.61 g, 9.69 mmol) and acetic acid (0.277 mL, 4.84 mmol) in MeOH (21 mL) was stirred at 0° C. for 75 min under N₂-atmosphere. Sodium cyanoborohydride (0.913 g, 14.5 mmol) was added in portions and the reaction was stirred at r.t. o.n. Thereafter MeOH (containing 1% NH₃) was added to adjust the pH to 7 and then the solvent was removed in vacuo. The crude product was purified by silica flash chromatography using DCM:[DCM:MeOH:NH₃=80:20:1]=100:0 to 40:60 as gradient to give the title compound (756 mg, 24%). ¹H NMR (400 MHz, CDCl₃) δ ppm 2.34 (s, 3H) 2.50 (br. s., 3H) 2.77 (br. s., 2H) 3.72-3.86 (m, 2H) 5.07-5.16 (m, 1H) 7.16 (dd, 1H) 7.33 (d, 1H) 7.48 (dd, 1H) 7.88 (dt, 1H) 8.36-8.40 (m, 1H). MS m/z 326.0 [M+H]⁺.

### Example 16d

### 8-Chloro-4-methyl-2-(3-methylpyridin-2-yl)-2,3,4,5-tetrahydropyrido[3,2-f][1,4]oxazepine

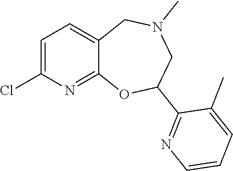

Sodium tert-butoxide (0.334 g, 3.48 mmol) was added in two portions to a stirred solution of 2-(((2,6-dichloropyridin-3-yl)methyl)(methyl)amino)-1-(3-methylpyridin-2-yl)ethanol (Example 16c, 0.756 g, 2.32 mmol) in THF (10 mL) at 0° C. The mixture was set under N₂-atmosphere, stirred for 5 min at 0° C. and was then allowed to warm-up to r.t. and stirred o.n. The solvent was removed in vacuo and the residue was treated with sat. NaHCO₃ solution and EtOAc and the phases were separated. The organic layer was dried over anhydrous sodium sulfate, filtered and concentrated. The crude was purified on a silica column using DCM:(DCM:MeOH:NH₃=90:10:1)=100:0 to 50:50 gradient to give the title compound (132 mg, 20%). ¹H NMR (400 MHz, CDCl₃) δ ppm 2.44 (s, 3H) 2.51-2.55 (m, 3H) 3.22 (dt, 1H) 3.61 (dd, 1H) 3.72 (dd, 1H) 3.94 (d, 1H) 5.29-5.33 (m, 1H) 7.09 (d, 1H) 7.18 (dd, 1H) 7.50-7.53 (m, 1H) 7.54 (d, 1H) 8.48 (dd, 1H). MS m/z 290.5 [M+H]⁺.

### Example 17

### 2-Cyclopropyl-N-(6-methoxy-5-(1-methyl-1H-pyrazol-4-yl)pyridin-2-yl)-4,5-dimethyl-2,3,4,5-tetrahydropyrido[3,2-f][1,4]oxazepin-8-amine; isomer 1

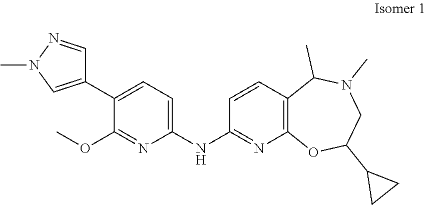

2-Chloro-8-cyclopropyl-5,6-dimethyl-5,6,7,8-tetrahydro-9-oxa-1,6-diaza-benzocycloheptene (Example 17 g, 122 mg, 0.48 mmol), 6-methoxy-5-(1-methyl-1H-pyrazol-4-yl)pyridin-2-ylamine (Example 2a, 99 mg, 0.48 mmol), Pd₂(dba)₃ (11.05 mg, 0.01 mmol), BINAP (15.0 mg, 0.02 mmol) and sodium tert-butoxide (70 mg, 0.72 mmol) were weighed in to a microwave vial, the vial was capped and flushed with argon. Toluene (4 mL) was added and the mixture was heated to 100° C. in a microwave apparatus for 2 h. The cooled reaction mixture was diluted with DCM and filtered. The mixture was concentrated and purified by column chromatography on silica, using gradient elution with increasing concentration of MeOH, from 0 to 8%, in DCM to give a mixture of the two diastereomers. This mixture was then subjected to SFC chromatography [Column: Chiralpak AD-H (21.2*250 mm); Mobile phase: 30% IPA+0.1% DEA: 70% CO₂; Flow 50 mL/min] to give isomer 1 (31 mg, 15%) which was the first isomer to elute. ¹H NMR (500 MHz, DMSO-d₆) δ ppm 2.14 (s, 3H) 2.37 (s, 3H) 2.38 (s, 3H) 2.99-3.06 (m, 1H) 3.46 (s, 1H) 3.66-3.81 (m, 2H) 3.95 (s, 3H) 5.33-5.37 (m, 1H) 7.04 (d, 1H) 7.07 (s, 1H) 7.30 (s, 1H) 7.64-7.71 (m, 3H) 7.79 (d, 1H) 9.96 (s, 1H). MS m/z 464.2 [M+H]⁺.

### Example 17a

### 2,6-Dichloro-N-methoxy-N-methyl-nicotinamide

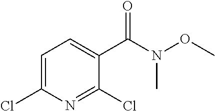

TEA (4.81 g, 47.5 mmol) was added to an ice cold solution of O,N-dimethyl-hydroxylamine hydrochloride in anhydrous DCM (200 mL), followed by slow addition of 2,6-dichloro-nicotinoyl chloride (CAS 58584-83-1, 5.0 g, 23.8 mmol) in DCM (20 mL). The reaction mixture was allowed to warm to r.t. and stirred for 1 h. The reaction mixture was quenched with water (5 mL), concentrated under reduced pressure and the residue was taken up in EtOAc (50 mL). The organic phase was washed with water, brine, dried over Na₂SO₄, filtered and concentrated in vacuo to afford the title compound (5.58 g, quantitative) which was used in the next step without further purification. ¹H NMR (400 MHz, CDCl₃) δ ppm 3.39 (br. s., 3H) 3.51 (br. s., 3H) 7.34 (d, 1H) 7.65 (d, 1H). MS m/z 235.0 [M+H]⁺.

### Example 17b

### 1-(2,6-Dichloro-pyridin-3-yl)-ethanone

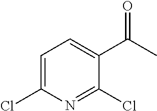

A solution of methylmagnesium bromide in ether (3.0 M, 9.36 mL, 28.1 mmol) was added dropwise to a solution of 2,6-dichloro-N-methoxy-N-methyl-nicotinamide (Example 17a, 5.5 g, 23.4 mmol) in anhydrous THF (100 mL) at −5° C. under a nitrogen atmosphere. The reaction mixture was stirred at −5° C. for 2 h, thereafter quenched with saturated solution of NH₄Cl and allowed to warm to r.t. The solids were removed by filtration and the filtrate was concentrated under reduced pressure. The residue was immediately purified on a small plug of silica gel using a gradient of 0 to 10% EtOAc in hexane to afford the title compound (4.1 g, 92%). ¹H NMR (400 MHz, CDCl₃) δ ppm 2.71 (s, 3H) 7.37 (d, 1H) 7.92 (d, 1H).

### Example 17c

### 1-(2,6-Dichloro-pyridin-3-yl)-ethanol

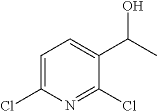

Sodium borohydride (0.816 g, 21.6 mmol) was added in small portions to a solution of 1-(2,6-dichloro-pyridin-3-yl)-ethanone (Example 17b, 4.1 g, 21.6 mmol) in MeOH (100 mL) at −10° C. The reaction mixture was allowed to warm to r.t. over 2 h. The reaction was then quenched with water (1 mL) and solvents were removed under reduced pressure. The residue was purified by flash column chromatography using 10% EtOAc in hexane to afford the title compound (4.15 g, quantitative). ¹H NMR (400 MHz, CDCl₃) δ ppm 1.50 (d, 3H) 5.21 (qd, 1H) 7.32 (d, 1H) 7.94 (d, 1H).

### Example 17d

### 3-(1-Bromo-ethyl)-2,6-dichloro-pyridine

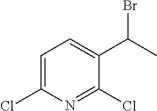

Triphenyl phosphine (3.0 g, 11.5 mmol) and N-bromosuccinimide (2.04 g, 11.5 mmol) were added to a solution of 1-(2,6-dichloro-pyridin-3-yl)-ethanol (Example 17c, 2.0 g, 10.6 mmol) in DCM (100 mL) at 0° C. under a nitrogen atmosphere. The reaction mixture was stirred at 0° C. for 2 h and concentrated under reduced pressure. The residue was purified by flash column chromatography using 5% EtOAc in hexane to afford the title compound (2.3 g, 86%) which was used immediately in the next step. ¹H NMR (400 MHz, CDCl₃) δ ppm 2.02 (d, 3H) 5.48 (q, 1H) 7.28-7.39 (m, 1H) 7.94 (d, 1H). MS m/z 256.0 [M+H]⁺.

### Example 17e

### 1-Cyclopropyl-2-[1-(2,6-dichloro-pyridin-3-yl)-ethylamino]-ethanol

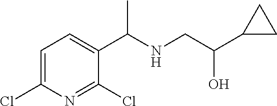

2-Amino-1-cyclopropyl-ethanol (CAS 54120-02-4, 1.37 g, 13.5 mmol) was added to mixture of cesium carbonate (5.86 g, 18.0 mmol) and 3-(1-bromo-ethyl)-2,6-dichloro-pyridine (Example 17d, 2.3 g, 9.0 mmol) in anhydrous DMF (50 mL) at r.t. under a nitrogen atmosphere. The reaction mixture was stirred o.n., filtered and concentrated under reduced pressure. The residue was purified by flash column chromatography using a gradient of 0% to 5% MeOH in DCM to obtain the title compound (1.35 g, quantitative). MS m/z 275.1 [M+H]⁺.

### Example 17f

### 2-Chloro-8-cyclopropyl-5-methyl-5,6,7,8-tetrahydro-9-oxa-1,6-diaza-benzocycloheptene

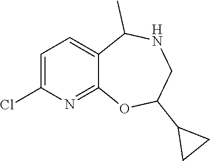

Sodium hydride (95% powder, 0.214 g, 8.47 mmol) was added to a solution of 1-cyclopropyl-2-[1-(2,6-dichloro-pyridin-3-yl)-ethylamino]-ethanol (Example 17e, 2.12 g, 7.7 mmol) in anhydrous THF (100 mL) at −40° C. The reaction mixture was allowed to warm to r.t. and stirred o.n. The reaction was quenched with sat. NH₄Cl (5.0 mL) and concentrated under reduced pressure. The residue was taken up in EtOAc (50 mL) and the organic phase was washed with water and brine, dried over Na₂SO₄, and concentrated under reduced pressure. The residue was purified by flash column chromatography using 5% MeOH in DCM to afford the title compound (1.82 g, quantitative) (mixture of diastereomers). Diastereomer 1: ¹H NMR (400 MHz, CDCl₃) δ ppm 0.28-0.41 (m, 1 H) 0.54-0.76 (m, 3H) 1.01-1.14 (m, 1H) 1.51 (d, 4H) 3.09-3.18 (m, 1H) 3.19-3.30 (m, 1H) 3.31-3.42 (m, 1H) 4.01 (q, 1H) 7.05 (d, 1H) 7.49 (d, 1H). Diastereomer 2: ¹H NMR (400 MHz, CDCl₃) δ ppm 0.27-0.42 (m, 1H) 0.52-0.76 (m, 3H) 0.96-1.13 (m, 1H) 1.48 (d, 3H) 1.61 (br. s., 1H) 3.17-3.28 (m, 1H) 3.31-3.48 (m, 2H) 4.10 (q, 1H) 7.00 (d, 1H) 7.42 (d, 1H). MS m/z 239.1 [M+H]⁺.

### Example 17g

### 2-Chloro-8-cyclopropyl-5,6-dimethyl-5,6,7,8-tetrahydro-9-oxa-1,6-diaza-benzocycloheptene

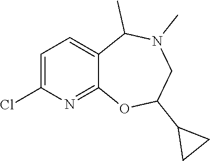

A mixture of 2-chloro-8-cyclopropyl-5-methyl-5,6,7,8-tetrahydro-9-oxa-1,6-diaza-benzocycloheptene (Example 17f, 0.80 g, 3.4 mmol) and paraformaldehyde (4.0 g) in MeOH was stirred at r.t. for 1 h under a nitrogen atmosphere. Sodium triacetoxyborohydride (3.55 g) was added and the resulting slurry was stirred at r.t. for 24 h. The solid was filtered off and the filtrate was concentrated under reduced pressure. The residue was purified by flash column chromatography using MeOH in DCM (a gradient of 0% to 5%) to afford the title compound (0.847 g, quantitative). ¹H NMR (400 MHz, CDCl₃) δ ppm 0.25-0.50 (m, 1H) 0.52-0.83 (m, 2H) 0.71-0.84 (m, 1H) 0.99-1.19 (m, 1H) 1.50 (d, 3H) 2.10-2.26 (m, 1H) 2.97-3.29 (m, 1H) 3.31-3.64 (m, 2H) 3.90-4.40 (m, 1H) 7.07 (dd, 1H) 7.39-7.60 (m, 1H). MS m/z 253.2 [M+H]⁺.

### Example 18

### 2-Cyclopropyl-N-(6-methoxy-5-(1-methyl-1H-pyrazol-4-yl)pyridin-2-yl)-4,5-dimethyl-2,3,4,5-tetrahydropyrido[3,2-f][1,4]oxazepin-8-amine; isomer 2

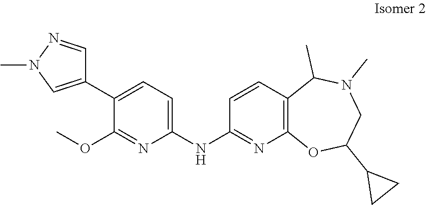

Preparation and separation as in Example 17 gave isomer 2 (31.6 mg, 16%), the second isomer to elute. ¹H NMR (500 MHz, DMSO-d₆) δ ppm 2.14 (s, 3H) 2.37 (s, 3H) 2.38 (s, 3H) 2.99-3.06 (m, 1H) 3.46 (s, 1H) 3.66-3.81 (m, 2H) 3.95 (s, 3H) 5.33-5.37 (m, 1H) 7.04 (d, 1H) 7.07 (s, 1 H) 7.30 (s, 1H) 7.64-7.71 (m, 3H) 7.79 (d, 1H) 9.96 (s, 1H). MS m/z 464.2 [M+H]⁺.

### Example 19

### 2-Cyclopropyl-N-(6-methoxy-5-(1-methyl-1H-pyrazol-4-yl)pyridin-2-yl)-4,5-dimethyl-2,3,4,5-tetrahydropyrido[3,2-f][1,4]oxazepin-8-amine; isomer 3

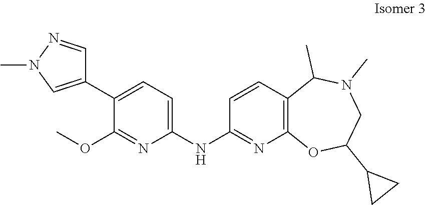

Preparation and separation as in Example 17 gave isomer 3 (29 mg, 14%), the third isomer to elute. ¹H NMR (500 MHz, CDCl₃) δ ppm 0.34 (m, 1H) 0.62 (m, 2H) 0.76 (m, 1H) 1.12 (m, 1H) 1.52 (d, 3H) 2.46 (s, 3H) 2.96 (d, 1H) 3.43 (m, 1H) 3.59 (m, 1H) 3.79 (m, 1H) 3.95 (s, 3H) 4.07 (s, 3 H) 6.69 (d, 1H) 7.15 (s, 1H) 7.41 (d, 1H) 7.64 (d, 1H) 7.70 (d, 1H) 7.79 (s, 1H) 7.82 (s, 1H). MS m/z 464.2 [M+H]⁺.

### Example 20

### 2-Cyclopropyl-N-(6-methoxy-5-(1-methyl-1H-pyrazol-4-yl)pyridin-2-yl)-4,5-dimethyl-2,3,4,5-tetrahydropyrido[3,2-f][1,4]oxazepin-8-amine; isomer 4

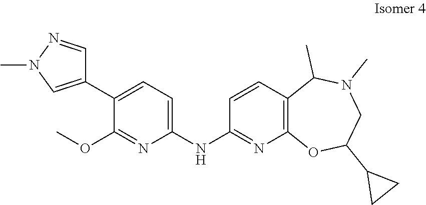

Preparation and separation as in Example 17 gave isomer 4 (29.7 mg, 15%), the fourth isomer to elute. ¹H NMR (500 MHz, CDCl₃) δ ppm 0.31 (m, 1H), 0.63 (m, 2H), 0.74 (m, 1H), 1.09 (m, 1 H), 1.52 (br. s., 3H), 2.22 (br. s., 3H), 3.20 (m, 1H), 3.39 (m, 2H), 3.95 (s, 3H), 4.08 (s, 3H), 4.29 (m, 1H), 6.69 (d, 1H), 7.16 (s, 1H), 7.48 (d, 1H), 7.71 (m, 2H), 7.80 (s, 1H), 7.82 (s, 1H). MS m/z 464.2 [M+H]⁺.

### Example 21

### N-(6-Methoxy-5-(1-methyl-1H-pyrazol-4-yl)pyridin-2-yl)-4,6-dimethyl-2-(4-methylthiazol-2-yl)-2,3,4,5-tetrahydropyrido[3,2-f][1,4]oxazepin-8-amine; isomer 1

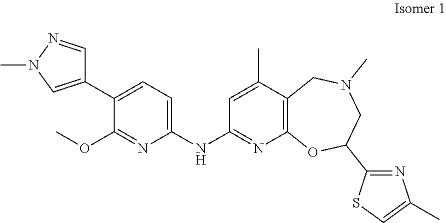

8-Chloro-4,6-dimethyl-2-(4-methylthiazol-2-yl)-2,3,4,5-tetrahydropyrido[3,2-f][1,4]oxazepine (Example 21c, 185 mg, 0.60 mmol), 6-methoxy-5-(1-methyl-1H-pyrazol-4-yl)pyridin-2-ylamine (Example 2a, 122 mg, 0.60 mmol), Pd₂(dba)₃ (13.7 mg, 0.01 mmol), BINAP (18.59 mg, 0.03 mmol) and sodium tert-butoxide (92 mg, 0.96 mmol) were weighed into a microwave vial. Toluene (5 mL) was added and the vial was capped and flushed with argon. The mixture was heated to 100° C. in a microwave apparatus for 2 h. The reaction mixture was diluted with DCM and filtered. The solvents were evaporated and the residue was purified by column chromatography on silica using a gradient of increasing concentration of MeOH, from 0 to 8%, in DCM to give 163 mg of the title product. The enantiomers were separated by SFC chromatography [Column: Chiralpak AD-H (21.2*250 mm); Mobile phase: 40% EtOH+0.1% DEA; 60% CO₂; Flow: 50 mL/min] to give N-(6-methoxy-5-(1-methyl-1H-pyrazol-4-yl)pyridin-2-yl)-4,6-dimethyl-2-(4-methylthiazol-2-yl)-2,3,4,5-tetrahydropyrido[3,2-f][1,4]oxazepin-8-amine, isomer 1 (66.3 mg, 23%), the first isomer to elute. ¹H NMR (500 MHz, DMSO-d₆) δ ppm 2.34 (s, 3H), 2.37 (s, 3H), 2.40 (s, 3H), 2.98-3.09 (m, 1 H), 3.42 (d, 1H), 3.66 (d, 1H), 3.85 (s, 3H), 3.90 (d, 1H), 4.03 (s, 3H), 5.28 (m, 1H), 6.94 (d, 1H), 7.30 (s, 1H), 7.82 (s, 1H), 7.83-7.86 (m, 2H), 8.02 (s, 1H), 9.64 (s, 1H). MS m/z 478.7 [M+H]⁺.

### Example 21a

### 2-((2,6-Dichloro-4-methylpyridin-3-yl)methylamino)-1-(4-methylthiazol-2-yl)ethanol

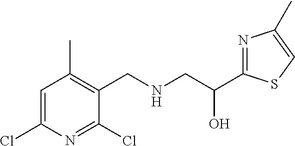

Potassium carbonate (1.529 g, 11.06 mmol) was added to a stirred solution of 3-(bromomethyl)-2,6-dichloro-4-methylpyridine (Example 1b, 1.128 g, 4.42 mmol) and 2-amino-1-(4-methylthiazol-2-yl)ethanol (Example 9b, 0.7 g, 4.42 mmol) in MeCN (35 mL) at r.t. The reaction mixture was stirred for 4 h, filtrated and the filtrate was concentrated. The crude product was purified by silica flash chromatography using DCM:[DCM:MeOH:NH₃=90:10:1]=100:0 to 40:60 as gradient to give the title compound (1.33 g, 90%). ¹H NMR (400 MHz, CDCl₃) δ ppm 2.38-2.44 (m, 3H) 2.44-2.49 (m, 3H) 3.12-3.29 (m, 2H) 3.97 (s, 2H) 5.06 (dd, 1H) 6.85 (d, 1H) 7.13 (s, 1H). MS m/z 333.9 [M+H]⁺.

### Example 21b

### 8-Chloro-6-methyl-2-(4-methylthiazol-2-yl)-2,3,4,5-tetrahydropyrido[3,2-f][1,4]oxazepine

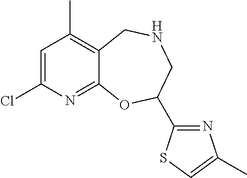

Sodium tert-butoxide (0.435 g, 4.53 mmol) was added in two portions to a stirred solution of 2-((2,6-dichloro-4-methylpyridin-3-yl)methylamino)-1-(4-methylthiazol-2-yl)ethanol (Example 21a, 1.157 g, 3.48 mmol) in 2-methyl-THF (25 mL) at 0° C. The mixture was set under N₂ atmosphere and stirred for 6 h at 45° C. Water was added to the cold reaction mixture and the phases were separated. The aqueous layer was extracted with EtOAc. The organic layer was dried over anhydrous sodium sulfate and concentrated. The residue was purified silica column using DCM:(DCM:MeOH:NH₃=90:10:1)=100:0 to 50:50 gradient, 40 g column to afford the title product (530 mg, 51%). ¹H NMR (400 MHz, CDCl₃) δ ppm 2.36 (s, 3H) 2.42-2.46 (m, 3H) 3.36 (dd, 1 H) 3.78 (dd, 1H) 3.92 (d, 1H) 4.18 (d, 1H) 5.22 (dd, 1H) 6.92 (d, 1H) 6.98 (s, 1H). MS m/z 296.6 [M+H]⁺.

### Example 21c

### 8-Chloro-4,6-dimethyl-2-(4-methylthiazol-2-A-2,3,4,5-tetrahydropyrido[3,2-f][1,4]oxazepine

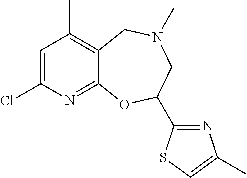

Formaldehyde 37% (1.31 mL, 17.6 mmol) and acetic acid (0.050 mL, 0.88 mmol) were added to a solution of 8-chloro-6-methyl-2-(4-methylthiazol-2-yl)-2,3,4,5-tetrahydropyrido[3,2-f][1,4]oxazepine (Example 21b, 0.52 g, 1.76 mmol) in MeOH (7 mL) at r.t. under N₂ atmosphere. The mixture was stirred for 15 min and then sodium cyanoborohydride (0.166 g, 2.64 mmol) was added and allowed to stir for 1 h at r.t. MeOH (containing 1% NH₃) was added to adjust the pH to 7 then the solvent was removed in vacuo. Sat. NaHCO₃ solution and EtOAc were added to the crude oil and the phases were separated. The aqueous layer was dried over anhydrous sodium sulfate, filtered and concentrated. The crude product was purified by silica flash chromatography using DCM:[DCM:MeOH:NH₃=90:10:1]=100:0 to 55:45 as gradient which gave the title compound (340 mg, 62%). ¹H NMR (400 MHz, CDCl₃) δ ppm 2.41 (br. s., 3H) 2.45 (d, 3H) 2.47-2.59 (m, 3H) 3.11-3.33 (m, 1H) 3.53-3.65 (m, 1H) 3.75-3.90 (m, 1H) 3.93-4.11 (m, 1H) 5.25-5.43 (m, 1H) 6.93 (s, 1H) 7.04 (s, 1H). MS m/z 310.6 [M+H]⁺.

### Example 22

### N-(6-Methoxy-5-(1-methyl-1H-pyrazol-4-yl)pyridin-2-yl)-4,6-dimethyl-2-(4-methylthiazol-2-yl)-2,3,4,5-tetrahydropyrido[3,2-f][1,4]oxazepin-8-amine; isomer 2

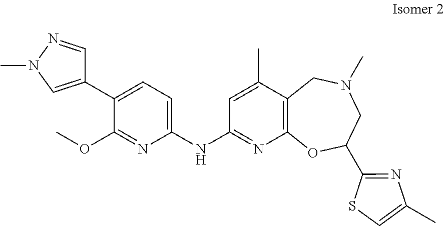

Synthesis and purification as in Example 21 to give N-(6-methoxy-5-(1-methyl-1H-pyrazol-4-yl)pyridin-2-yl)-4,6-dimethyl-2-(4-methylthiazol-2-yl)-2,3,4,5-tetrahydropyrido[3,2-f][1,4]oxazepin-8-amine, enantiomer 2 (65.2 mg, 23%) the second isomer to elute. ¹H NMR (500 MHz, DMSO-d₆) δ ppm 2.34 (s, 3H), 2.37 (s, 3H), 2.40 (s, 3H), 2.98-3.09 (m, 1H), 3.42 (d, 1 H), 3.66 (d, 1H), 3.85 (s, 3H), 3.90 (d, 1H), 4.03 (s, 3H), 5.28 (m, 1H), 6.94 (d, 1H), 7.30 (s, 1 H), 7.82 (s, 1H), 7.83-7.86 (m, 2H), 8.02 (s, 1H), 9.64 (s, 1H). MS m/z 478.7 [M+H]⁺.

### Example 23

### 2-(3-Chlorophenyl)-4-methyl-N-(5-(3-methyl-1H-1,2,4-triazol-1-yl)pyridin-2-yl)-2,3,4,5-tetrahydropyrido[3,2-f][1,4]oxazepin-8-amine

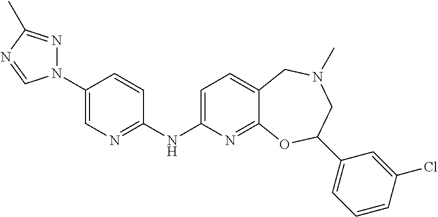

Acetoxy(2′-(di-tert-butylphosphino)biphenyl-2-yl)palladium (8.59 mg, 0.02 mmol) was reduced to Pd(0) by heating in DME (2 mL) and water (0.100 mL) together with cesium carbonate (181 mg, 0.56 mmol) for 5 min at 85° C. 8-Chloro-2-(3-chlorophenyl)-4-methyl-2,3,4,5-tetrahydropyrido[3,2-f][1,4]oxazepine (Example 23d, 115 mg, 0.37 mmol) and 5-(3-methyl-1H-1,2,4-triazol-1-yl)pyridin-2-amine (Example 23a. 65 mg, 0.37 mmol) were added to the microwave vial. The reaction mixture was run in the microwave oven for 180 minutes at 100° C. The solids were filtered off and washed with DCM. The solvent was removed and the crude product was purified using flash chromatography, 0-10% MeOH in DCM yielding the title compound (32.0 mg, 19%). ¹H NMR (500 MHz, DMSO-d₆) δ ppm 2.36 (d, 6H) 2.95-3.08 (m, 2H) 3.67-3.81 (m, 2H) 5.14-5.20 (m, 1H) 7.39-7.49 (m, 3H) 7.53-7.59 (m, 2H) 7.65 (s, 2H) 8.03 (dd, 1H) 8.63 (d, 1H) 9.00 (s, 1H) 10.03 (s, 1H). MS m/z 448.1 [M+H]⁺.

### Example 23a

### 5-(3-Methyl-1H-1,2,4-triazol-1-yl)pyridin-2-amine

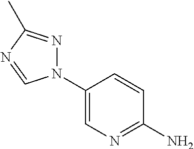

CuI (285 mg, 20 mol %), L-proline (345 mg, 40 mol %) and potassium carbonate₃ (2.07 g, 15.00 mmol) were added to a solution of 5-bromo-pyridin-2-ylamine (CAS 1072-97-5, 1.30 g, 7.50 mmol) and 3-methyl-1H-[1,2,4]triazole (CAS 7170-01-6, 935 mg, 11.3 mmol) in anhydrous DMSO (5 mL). The reaction mixture was heated at 120° C. for 5 days, cooled to r.t., diluted with DCM and filtered through a pad of Celite. The filtrate was concentrated under reduced pressure and the residue was purified by a reversed phase purification (C18-column) using stepped gradients of 20 to 50% MeOH in water (containing 1% NH₄OH) to give the title product (153 mg, 12%). ¹H NMR (400 MHz, MeOD) δ ppm 2.41 (s, 3H) 6.67 (d, 1H) 7.75 (dd, 1H) 8.24 (d, 1H) 8.72 (s, 1H). MS (ES) m/z 176.2 [M+H]⁺.

### Example 23b

### 1-(3-Chlorophenyl)-2-(methylamino)ethanol

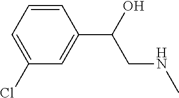

To 2-(3-chlorophenyl)oxirane (895 mg, 5.79 mmol) was added methanamine (434 μL, 34.7 mmol), 8M in MeOH. The reaction was heated in the microwave oven to 85° C. for 1 h. The solvent was removed, yielding the title compound (1100 mg, quantitative). MS (ES+) m/z 186.6 [M+H]⁺.

### Example 23c

### 1-(3-Chlorophenyl)-2-(((2,6-dichloropyridin-3-yl)methyl)(methyl)amino)ethanol

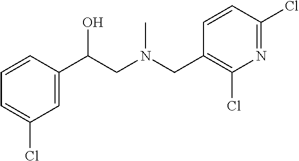

To 1-(3-chlorophenyl)-2-(methylamino)ethanol (0.705 g, 3.8 mmol) in acetonitrile (10 mL) were added 3-(bromomethyl)-2,6-dichloropyridine (0.915 g, 3.80 mmol) and potassium carbonate (0.630 g, 4.56 mmol). The reaction was stirred for 16 h. The solvent was removed and the crude product was partitioned between water and DCM before purifying it on silica, 0-30% EtOAc in heptane yielding the title compound (1.184 g, 90%).

### Example 23d

### 8-Chloro-2-(3-chlorophenyl)-4-methyl-2,3,4,5-tetrahydropyrido[3,2-f][1,4]oxazepine

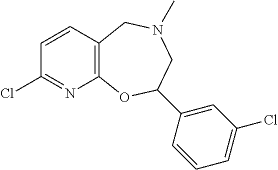

To 1-(3-chlorophenyl)-2-(((2,6-dichloropyridin-3-yl)methyl)(methyl)amino)ethanol (1.184 g, 3.43 mmol) in THF (10 mL) was added sodium hydride (0.178 g, 4.45 mmol). The reaction was heated to 50° C. for 1 h. Water and DCM were carefully added and the crude product was shaken into the organic phase. The solvents were evaporated and the crude product was used as such (1.080 g, quantitative). ¹H NMR (500 MHz, DMSO-d₆) δ ppm 2.36 (s, 3H) 2.99-3.10 (m, 2H) 3.77-3.89 (m, 2H) 5.24 (dd, 1H) 7.27 (d, 1H) 7.40-7.46 (m, 3H) 7.54 (s, 1H) 7.82 (d, 1H). MS (ES+) m/z 309.6 [M+H]⁺.

### Example 24

### 2-(4-Chlorophenyl)-4-methyl-N-(5-(3-methyl-1H-1,2,4-triazol-1-yl)pyridin-2-yl)-2,3,4,5-tetrahydropyrido[3,2-f][1,4]oxazepin-8-amine

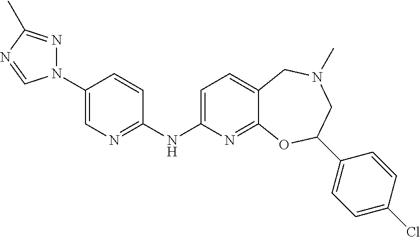

Preparation in analogy with Example 23, using 8-chloro-2-(4-chlorophenyl)-4-methyl-2,3,4,5-tetrahydropyrido[3,2-f][1,4]oxazepine (Example 24c, 115 mg, 0.37 mmol) and 5-(3-methyl-1H-1,2,4-triazol-1-yl)pyridin-2-amine (Example 23a, 65 mg, 0.37 mmol) as starting materials to give the title compound (63.0 mg, 38%). ¹H NMR (500 MHz, DMSO-d₆) δ ppm 2.36 (d, 6H) 2.93-3.05 (m, 2H) 3.67-3.80 (m, 2H) 5.15 (dd, 1H) 7.45-7.58 (m, 5H) 7.65 (s, 2H) 8.02 (dd, 1H) 8.62 (d, 1H) 9.00 (s, 1H) 10.04 (s, 1H). MS m/z 448.2 [M+H]⁺.

### Example 24a

### 1-(4-Chlorophenyl)-2-(methylamino)ethanol

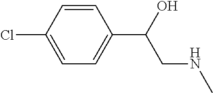

To 2-(4-chlorophenyl)oxirane (2.0 g, 12.94 mmol) was added methanamine (12.9 mL, 103 mmol), 8 M in MeOH. The reaction was heated in the microwave reactor for 1 h at 85° C. The solvent was removed yielding the title compound (2.5 g, quantitative). MS (ES+) m/z 186.6 [M+H]⁺.

### Example 24b 1-(4-chlorophenyl)-2-(((2,6-dichloropyridin-3-yl)methyl)(methyl)amino)ethanol

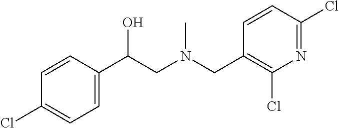

To 1-(4-chlorophenyl)-2-(methylamino)ethanol (1.57 g, 8.45 mmol) in acetonitrile (20 mL) were added 3-(bromomethyl)-2,6-dichloropyridine (2.04 g, 8.45 mmol) and potassium carbonate (1.40 g, 10.1 mmol). The reaction was stirred for 16 h. The solvent was removed and the crude product was partitioned between water and DCM before purifying it on silica, 0-30% EtOAc in heptane yielding the title compound (2.80 g, 96%). ¹H NMR (500 MHz, DMSO-d₆) δ ppm 2.27 (s, 3H) 2.51-2.54 (m, 1H) 2.58-2.64 (m, 1H) 3.63 (s, 2H) 4.74 (dt, 1H) 5.27 (d, 1H) 7.30-7.37 (m, 4H) 7.48 (d, 1 H) 7.84 (d, 1H). MS (ES+) m/z 345.6 [M+H]⁺.

### Example 24c

### 8-Chloro-2-(4-chlorophenyl)-4-methyl-2,3,4,5-tetrahydropyrido[3,2-f]-[1,4]oxazepine

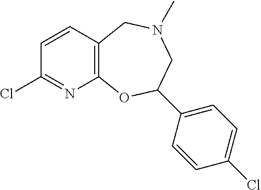

To 1-(4-chlorophenyl)-2-(((2,6-dichloropyridin-3-yl)methyl)(methyl)amino)ethanol (Example 24b, 2.79 g, 8.09 mmol) in THF (10 mL) was added sodium hydride (0.42 g, 10.5 mmol). The reaction was heated to 50° C. for 1 h. Water and DCM were carefully added and the crude product was shaken into the organic phase. The solvents were evaporated and the crude product was used as such: Yield: (2.41 g, 96%). ¹H NMR (500 MHz, DMSO-d₆) δ ppm 2.35 (s, 3H) 2.94-3.11 (m, 2H) 3.75-3.89 (m, 2H) 5.23 (dd, 1H) 7.26 (d, 1H) 7.44-7.51 (m, 4H) 7.81 (d, 1H). MS (ES+) m/z 309.6 [M+H]⁺.

### Example 25

### N-(6-Methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine; isomer 1

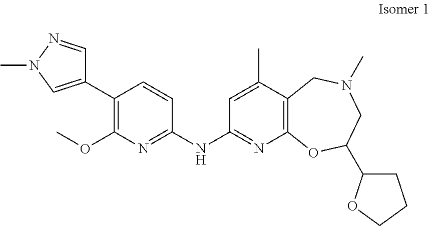

The major diastereomer of Example 25a was precipitated from MeOH once and from EtOH twice. The rest of the material was purified by HPLC to give N-(6-methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine, diastereomer 1, the minor stereomer (39 mg, 8.6%). ¹H NMR (500 MHz, CDCl₃) δ ppm 1.89-1.99 (m, 2H), 2.11-2.20 (m, 1H), 2.20-2.29 (m, 1H), 2.35 (s, 3H), 2.48 (s, 3H), 2.83 (m, 1H), 3.20 (d, 1H), 3.57 (d, 1H), 3.77-3.86 (m, 3H), 3.86-3.92 (m, 1H), 3.95 (s, 3H), 3.96-4.02 (m, 1H), 4.07 (s, 3H), 6.78 (d, 1H), 7.03 (s, 1H), 7.49 (s, 1H), 7.69 (d, 1H), 7.81 (s, 1H), 7.79 (s, 1H). MS m/z 451 [M+H]⁺.

### Example 25a

### N-(6-Methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine

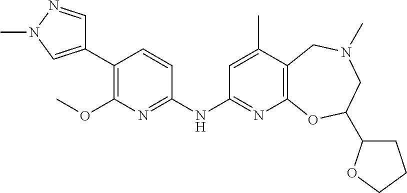

Preparation in analogy with Example 21, using 6-methoxy-5-(1-methyl-1H-pyrazol-4-yl)pyridin-2-ylamine (Example 2a, 205 mg, 1.00 mmol) and 8-chloro-4,6-dimethyl-2-(tetrahydrofuran-2-yl)-2,3,4,5-tetrahydropyrido[3,2-f][1,4]oxazepine (Example 25h, 284 mg, 1.00 mmol) as starting materials to give a mixture of two diastereomers which were purified by column chromatography on silica gel using gradient elution with increasing concentration of methanol, from 0 to 8%, in dichloromethane.

### Example 25b

### Tetrahydro-furan-2-carbaldehyde

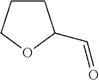

IBX (41.0 g, 146.9 mmol) was added to a solution of furfuryl alcohol (CAS 98-00-0, 5.0 g, 48.96 mmol) in MeCN (250 mL) at r.t. The reaction mixture was heated at 80° C. for 30 min and cooled to r.t. The solid was removed by filtration and the filtrate concentrated under reduced pressure to afford the crude tetrahydro-furan-2-carbaldehyde (3.4 g, 69%), which was used in the next step without further purification.

### Example 25c

### 2-Nitro-1-(tetrahydro-furan-2-yl)-ethanol

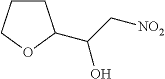

Nitromethane (5.0 mL) was added to a mixture of tetrahydro-furan-2-carbaldehyde (Example 25b, 1.0 g, 9.98 mmol) and DIPEA (2.6 g, 19.98 mmol) in anhydrous THF (10.0 mL) at 0° C. The reaction mixture was stirred for 30 min while allowing the reaction to warm to r.t. and the volatiles were removed under reduced pressure. The residue was taken in EtOAc (20 mL) and the organic phase was washed with 5% aqueous citric acid, 5% aqueous NaHCO₃ and water, dried over anhydrous Na₂SO₄, and concentrated under reduced pressure to afford the title compound (0.74 g, 46%). ¹H NMR (400 MHz, CDCl₃) δ ppm 4.65 (dd, 1H) 4.51-4.38 (m, 1H) 4.24 (ddd, 1H) 3.96-3.85 (m, 2H) 3.80 (dddd, 1H) 2.08-1.83 (m, 4H).

### Example 25d

### 2-Amino-1-(tetrahydro-furan-2-yl)-ethanol

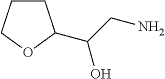

A mixture of 2-nitro-1-(tetrahydro-furan-2-yl)-ethanol (Example 25c, 8.84 g, 54.8 mmol) and palladium on activated carbon (10%, 0.884 g) in anhydrous MeOH (100 mL) was stirred under hydrogen atmosphere (40 psi) at r.t. o.n. The reaction mixture was filtered through a pad of Celite and concentrated under reduced pressure. The residue was purified by flash column chromatography using 0-7% MeOH in DCM to afford the title compound (2.8 g, 53%). ¹H NMR (400 MHz, MeOD) δ ppm 3.90-3.79 (m, 1H) 3.76-3.67 (m, 2H) 3.50-3.41 (m, 1H) 2.82 (dd, 1H) 2.59 (dd, 1H) 2.02-1.77 (m, 4H). MS m/z 132.2 [M+H]⁺.

### Example 25e

### 2-Amino-1-(tetrahydro-furan-2-yl)-ethanol hydrochloride

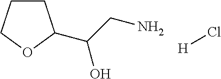

2-Amino-1-(tetrahydro-furan-2-yl)-ethanol (Example 25d, 2.22 g, 15.3 mmol) was dissolved in anhydrous diethyl ether (50 mL) and 2 M HCl in diethyl ether (15.3 mL, 30.5 mmol) was added at 0° C. The precipitated solid was collected by filtration and air-dried to afford the title compound (2.2 g, 76%). ¹H NMR (400 MHz, MeOD) δ ppm 3.93-3.81 (m, 1H) 3.81-3.69 (m, 2H) 3.61 (td, 1 H) 3.13 (dd, 1H) 2.86 (dd, 1H) 2.11-2.00 (m, 1H) 1.99-1.76 (m, 3H). MS m/z 132.1 [M+H]⁺. Elemental analysis: Calculated for C₆H_{14.15}C_{11.15}NO₂ C, 41.63; H, 8.24; N, 8.09. found C, 41.69; H, 8.32; N, 8.50.

### Example 25f

### 2-((2,6-Dichloro-4-methylpyridin-3-yl)methylamino)-1-(tetrahydrofuran-2-yl)ethanol

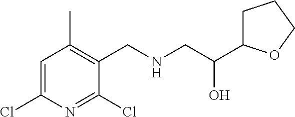

3-(Bromomethyl)-2,6-dichloro-4-methylpyridine (Example 1b, 1.56 g, 6.13 mmol) and TEA (1.92 mL, 13.8 mmol) were added to a suspension of 2-amino-1-(tetrahydrofuran-2-yl)ethanol hydrochloride (Example 25e, 1.06 g, 6.31 mmol) in MeCN (2 mL) and DMF (2 mL). The mixture was stirred at r.t. for 2.5 h. The solvent was evaporated at reduced pressure and the residue was dissolved in EtOAc, washed with water and brine and dried over MgSO₄. Evaporation and drying in vacuo gave the title compound (1.625 g, 87%). MS m/z 305.0 [M+H]⁺.

### Example 25g

### 2-(((2,6-Dichloro-4-methylpyridin-3-yl)methyl)(methyl)amino)-1-(tetrahydrofuran-2-yl)ethanol

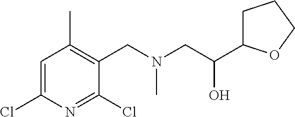

To a solution of 2-((2,6-dichloro-4-methylpyridin-3-yl)methylamino)-1-(tetrahydrofuran-2-yl)ethanol (Example 25f, 1.63 g, 5.32 mmol) in MeOH (25 mL) were sequentially added formaldehyde (1.47 mL, 53.2 mmol), acetic acid (0.381 mL, 6.66 mmol) and Sodium cyanoborohydride (0.418 g, 6.66 mmol). The resulting mixture was stirred at r.t. for 3 h. Saturated aqueous NaHCO₃ was added to the mixture and the solvent was evaporated. The aqueous residue was extracted with EtOAc, the combined extracts were dried over MgSO₄ and evaporated. The residue was purified by column chromatography on silica using gradient elution with increasing concentration of MeOH, from 0 to 10% in DCM to give the title compound (1.20 g, 71%). MS m/z 319.0 [M+H]⁺.

### Example 25h

### 8-Chloro-4,6-dimethyl-2-(tetrahydrofuran-2-yl)-2,3,4,5-tetrahydropyrido[3,2-f][1,4]oxazepine

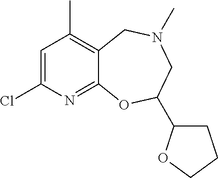

Sodium tert-butoxide (0.394 g, 4.10 mmol) was added to a solution of 2-(((2,6-dichloro-4-methylpyridin-3-yl)methyl)(methyl)amino)-1-(tetrahydrofuran-2-yl)ethanol (Example 25g, 1.19 g, 3.73 mmol) in THF (10 mL). The reaction mixture was heated to 40° C. for 16 h. The cooled reaction mixture was diluted with EtOAc and washed with water, dried over MgSO₄ and concentrated. The residue was purified by column chromatography on silica using gradient elution with increasing concentration of MeOH, from 0 to 10% in DCM to give 8-chloro-4,6-dimethyl-2-(tetrahydrofuran-2-yl)-2,3,4,5-tetrahydropyrido[3,2-f][1,4]oxazepine (0.859 g, 81%). ¹H NMR (500 MHz, CDCl₃) δ ppm 1.87-1.96 (m, 2H), 2.08-2.18 (m, 1H), 2.18-2.27 (m, 1H), 2.33 (s, 3H), 2.44-2.51 (m, 3 H), 2.80-3.00 (m, 1H), 3.12-3.22 (m, 1H), 3.52-3.65 (m, 1H), 3.73-3.91 (m, 4H), 3.94-4.00 (m, 1H), 6.92 (s, 1H). MS m/z 283.6 [M+H]⁺.

### Example 26

### N-(6-Methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine; isomer 2

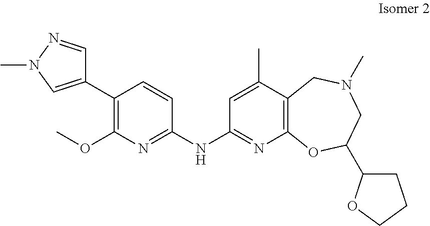

The major diastereomer of Example 25a was precipitated from MeOH once and from EtOH twice. The rest of the material was purified by HPLC. The combined yield of N-(6-methoxy-5-(1-methyl-1H-pyrazol-4-yl)pyridin-2-yl)-4,6-dimethyl-2-(tetrahydrofuran-2-yl)-2,3,4,5-tetrahydropyrido[3,2-f][1,4]oxazepin-8-amine, diastereomer 2 (the major isomer) from the precipitation and the HPLC purification was 161 mg, 35%. ¹H NMR (500 MHz, CDCl₃) δ ppm 1.89-1.99 (m, 2H), 2.11-2.20 (m, 1H), 2.20-2.29 (m, 1H), 2.35 (s, 3H), 2.48 (s, 3H), 2.83 (m, 1H), 3.20 (d, 1H), 3.57 (d, 1 H), 3.77-3.86 (m, 3H), 3.86-3.92 (m, 1H), 3.95 (s, 3H), 3.96-4.02 (m, 1H), 4.07 (s, 3H), 6.78 (d, 1H), 7.03 (s, 1H), 7.49 (s, 1H), 7.69 (d, 1H), 7.81 (s, 1H), 7.79 (s, 1H). MS m/z 454.2 [M+H]⁺.

### Example 27

### [6-Methoxy-5-(1-methyl-1H-pyrazol-4-yl)-pyridin-2-yl]-[6-methyl-8-(3,3,3-trifluoro-propyl)-5,6,7,8-tetrahydro-9-oxa-1,6-diaza-benzocyclohepten-2-yl]-amine

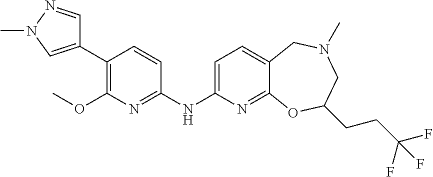

Palladium acetate (0.023 g, 5 mol %), Xantphos (0.118 g, 30 mol %) and cesium carbonate (0.33 g, 1.02 mmol) were added to a degassed mixture of 2-chloro-6-methyl-8-(3,3,3-trifluoro-propyl)-5,6,7,8-tetrahydro-9-oxa-1,6-diaza-benzocycloheptene (Example 27e, 0.2 g, 0.68 mmol), 6-methoxy-5-(1-methyl-1H-pyrazol-4-yl)-pyridin-2-ylamine (Example 2a, 0.139 g, 0.68 mmol) in anhydrous 1,4-dioxane (20 mL). The reaction mixture was purged with nitrogen for additional 20 min and then heated in a microwave reactor at 145° C. for 1 h. The reaction mixture was diluted with DCM (20 mL) and filtered through a small pad of Celite. The volatiles were removed under reduced pressure and the residue was purified by flash column chromatography using a gradient of 0 to 10% MeOH in DCM to afford the title compound (160 mg, 51%). ¹H NMR (400 MHz, CDCl₃) δ ppm 1.74-1.88 (m, 1H) 1.91-2.05 (m, 1H) 2.25-2.39 (m, 1H) 2.43 (s, 3H) 2.55-2.72 (m, 1H) 2.79-3.00 (m, 2H) 3.57 (d, 1H) 3.69 (d, 1H) 3.94 (s, 3H) 4.01 (s, 3H) 4.09-4.17 (m, 1H) 6.76 (d, 1 H) 7.10 (s, 1H) 7.44 (d, 1H) 7.57 (d, 1H) 7.70 (d, 1H) 7.81 (s, 1H) 7.78 (s, 1H). ESMS m/z 463.2 [M+H]⁺.

### Example 27a

### 5,5,5-Trifluoro-1-nitro-pentan-2-ol

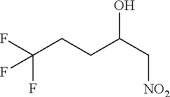

Freshly prepared sodium hydroxide solution (1.58 g, 39.7 mmol, in 10 mL of water) was added dropwise to a mixture of 4,4,4-trifluorobutyraldehyde (5.0 g, 39.7 mmol) and nitromethane (2.42 g, 39.66 mmol) in MeOH (50 mL) at −10° C. The reaction mixture was warmed to 0° C., stirred for 1 h and then quenched with acetic acid (5.0 mL). The volatiles were removed under reduced pressure and the residue was diluted with water (25 mL). The aqueous phase was neutralized with sodium bicarbonate (5.0 g) and extracted with ethyl acetate (3×50 mL). The combined organic extracts were dried over MgSO₄, filtered and concentrated under reduced pressure to afford the title compound (2.0 g, 27%) which was used in the next step without further purification. ¹H NMR (400 MHz, CDCl₃) δ ppm 1.68-1.84 (m, 2H) 2.15-2.33 (m, 1H) 2.35-2.51 (m, 1H) 2.76 (d, 1H) 4.35-4.41 (m, 3H). ESMS m/z 186.0 [M−1]⁻.

### Example 27b

### 1-Amino-5,5,5-trifluoro-pentan-2-ol

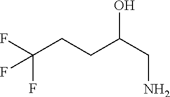

Pd/C (10 wt % wet, 0.2 g) was added to a solution of 5,5,5-trifluoro-1-nitro-pentan-2-ol (Example 27a, 2.0 g, 10.7 mmol) in MeOH (30 mL). The mixture was shaken under a hydrogen atmosphere (32 psi) at r.t. for 24 h. The mixture was filtered through a small plug of Celite and the filtrate was concentrated under reduced pressure to afford the title compound (1.52 g, 90%) which was used in the next step without further purification. ¹H NMR (400 MHz, CDCl₃) δ ppm 1.53-1.71 (m, 2H) 1.81 (br.s, 3H) 2.09-2.27 (m, 1H) 2.29-2.46 (m, 1H) 2.52 (dd, 1H) 2.90 (dd, 1H) 3.50-3.58 (m, 1H).

### Example 27c

### 1-[(2,6-Dichloro-pyridin-3-ylmethyl)-amino]-5,5,5-trifluoro-pentan-2-ol

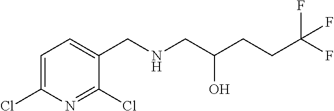

1-Amino-5,5,5-trifluoro-pentan-2-ol (Example 27b, 2.6 g, 15.56 mmol) was added to a mixture of cesium carbonate (6.08 g, 18.67 mmol) and 3-(bromomethyl)-2,6-dichloropyridine (CAS 58596-59-1, 3.73 g, 15.56 mmol) in anhydrous DMF (20 mL) at r.t. under a nitrogen atmosphere. The reaction mixture was stirred o.n., filtered and concentrated under reduced pressure. The residue was purified by flash column chromatography using a gradient of 0 to 5% MeOH in DCM to obtain the title compound (2.6 g, 53%). ESMS m/z 318.9 [M+H]⁺.

### Example 27d

### 2-Chloro-8-(3,3,3-trifluoro-propyl)-5,6,7,8-tetrahydro-9-oxa-1,6-diaza-benzocycloheptene

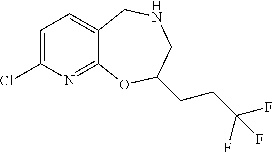

Sodium hydride (95% powder, 0.262 g, 9.84 mmol) was added to a solution of 1-[(2,6-dichloro-pyridin-3-ylmethyl)-amino]-5,5,5-trifluoro-pentan-2-ol (Example 27c, 2.6 g, 8.2 mmol) in anhydrous THF (50 mL) at −40° C. The reaction mixture was allowed to warm to r.t. and stirred o.n. The reaction was quenched with saturated solution of NH₄Cl (5.0 mL) and concentrated under reduced pressure. The residue was taken in ethyl acetate (50 mL) and the organic phase was washed with water and brine, dried over Na₂SO₄, and concentrated under reduced pressure. The residue was purified by flash column chromatography using a gradient of 0 to 3% MeOH in DCM to afford the title compound (2.4 g, quantitative, a mixture of enantiomers). ¹H NMR (400 MHz, CDCl₃) δ ppm 1.94 (s, 2H) 3.01-3.17 (m, 1H) 3.14-3.32 (m, 1H) 3.61-3.79 (m, 1H) 3.84-4.03 (m, 3H) 4.54 (d, 2H) 7.03 (d, 1H) 7.43 (d, 1H).

### Example 27e

### 2-Chloro-6-methyl-8-(3,3,3-trifluoro-propyl)-5,6,7,8-tetrahydro-9-oxa-1,6-diaza-benzocycloheptene

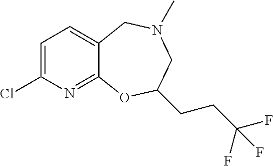

A mixture of 2-chloro-8-(3,3,3-trifluoro-propyl)-5,6,7,8-tetrahydro-9-oxa-1,6-diaza-benzocycloheptene (Example 27d, 2.6 g, 3.4 mmol) and paraformaldehyde (10.0 g) in MeOH (250 mL) was stirred at r.t. for 1 h under a nitrogen atmosphere. Sodium triacetoxyborohydride (8.5 g) was added and the resulting slurry was stirred at r.t. for 24 h. The precipitated solid was removed by filtration and the filtrate was concentrated under reduced pressure. The residue was purified by flash is column chromatography using a gradient of 0 to 5% MeOH in DCM to afford the title compound (1.39 g, 51%). ¹H NMR (400 MHz, CDCl₃) δ ppm 1.75-1.88 (m, 1H) 1.90-2.03 (m, 1H) 2.25-2.39 (m, 1H) 2.42 (s, 3H) 2.56-2.73 (m, 1H) 2.82-2.99 (m, 2H) 3.57-3.76 (m, 2H) 4.02-4.12 (m, 1H) 4.87-5.01 (m, 1H) 7.05 (d, 1H) 7.46 (d, 1H). ESMS m/z 295.0 [M+H]⁺.

### Example 28

### [6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine

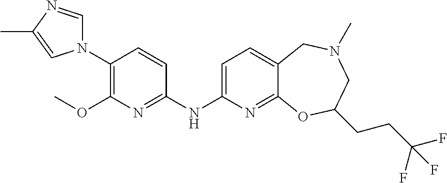

Palladium acetate (0.023 g, 5 mol %), Xantphos (0.118 g, 30 mol %) and cesium carbonate (0.33 g, 1.02 mmol) were added to a degassed mixture of 2-chloro-6-methyl-8-(3,3,3-trifluoro-propyl)-5,6,7,8-tetrahydro-9-oxa-1,6-diaza-benzocycloheptene (Example 27e, 0.2 g, 0.68 mmol), 6-s methoxy-5-(4-methyl-imidazol-1-yl)-pyridin-2-ylamine (Example 6a, 0.139 g, 0.68 mmol) in anhydrous 1,4-dioxane (20 mL). The reaction mixture was purged with nitrogen for 20 min and then heated in a microwave reactor at 145° C. for 1 h. The reaction mixture was diluted with DCM (20 mL) and filtered through a small pad of Celite. The volatiles were removed under reduced pressure and the residue was purified by flash column chromatography using a gradient of 0 to 10% MeOH in DCM to afford the title compound (172 mg, 55%). ¹H NMR (400 MHz, CDCl₃) δ ppm 1.82 (m, 1H) 1.90-2.06 (m, 1H) 2.30 (s, 2H) 2.44 (s, 3H) 2.55-2.71 (m, 1H) 2.80-3.01 (m, 2H) 3.59 (d, 1H) 3.70 (d, 1H) 3.99 (s, 3H) 4.08 (t, 1H) 6.81-6.98 (m, 2H) 7.26 (m, 2H) 7.29 (br. s., 1H) 7.41-7.53 (m, 3H) 7.63 (s, 1H). ESMS m/z 461.1 [M−1]³¹ .

### Example 29

### N-(6-Methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine; isomer 3

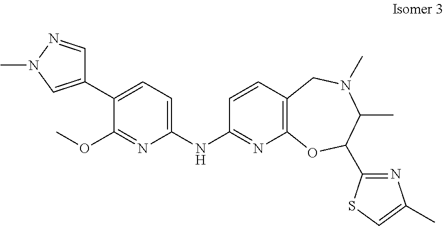

The isomers of N-(6-methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine (Example 29f, 147 mg, 0.31 mmol) were separated using SFC chromatography [Column: Chiralpak OJ-H (21.2*250 mm) Mobile phase: 20% EtOH+0.1% DEA; 80% CO₂, Flow: 50 mL/min] yielding N-(6-methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine, isomer 3 the third isomer to elute (24.5 mg, 17%). (Isomers 1 and 2 were not isolated) ¹H NMR (500 MHz, DMSO-d₆) δ ppm 1.02 (d, 3H) 2.37 (s, 3 H) 2.43 (s, 3H) 3.43-3.51 (m, 2H) 3.85 (s, 3H) 4.01 (s, 3H) 4.20 (d, 1H) 5.46 (d, 1H) 7.03 (d, 1 H) 7.25 (s, 1H) 7.63 (d, 1H) 7.79-7.87 (m, 3H) 8.02 (s, 1H) 9.72 (s, 1H). MS m/z 478.2 [M+H]⁻.

### Example 29a

### 2-Chloro-1-(4-methylthiazol-2-yl)propan-1-one

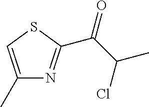

To n-BuLi (1.6 M in heptane, 18.8 mL, 30.1 mmol) dissolved in diethyl ether (35 mL) and cooled to −78° C., was slowly added 4-methylthiazole (CAS 693-95-8, 2.3 mL, 25.1 mmol) dissolved in diethyl ether (35 mL). Stirring was continued for 30 min before 2-chloro-1-morpholinopropan-1-one (CAS 54022-76-3, 4.46 g, 25.12 mmol) dissolved in toluene (10 mL) was added slowly. The reaction was allowed to retain r.t. and was stirred at r.t. for 1.5 h. The reaction was quenched with sat. NaHCO₃. The organic phase was dried over MgSO₄ and the solvent was then evaporated yielding 2-chloro-1-(4-methylthiazol-2-yl)propan-1-one (4.71 g, quantitative). ¹H NMR (500 MHz, CDCl₃) δ ppm 1.79 (d, 3H), 2.56 (d, 3H), 5.69 (q, 1H), 7.34 (d, 1H). MS m/z 189.9 [M+H]⁺.

### Example 29b

### 2-Chloro-1-(4-methylthiazol-2-yl)propan-1-ol

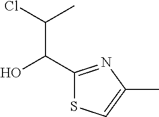

To 2-chloro-1-(4-methylthiazol-2-yl)propan-1-one (Example 29a, 4.71 g, 24.85 mmol) in MeOH (15 mL) was added sodium borohydride (1.410 g, 37.27 mmol) in portions at 0° C. The reaction mixture was stirred for 1.5 h. The crude product was partitioned between DCM and sat. NaHCO₃. The organic phase was separated and the solvent was removed. The crude product was purified by silica flash chromatography using a gradient of MeOH (0 to 5%) in DCM. Pure fractions were collected and impure fractions were pooled and purified again using a gradient of MeOH (0 to 3%) in DCM to give the title compound (2.09 g, 44%). ¹H NMR (500 MHz, CDCl₃) δ ppm 1.61 (d, 3 H), 2.45 (d, 4H), 3.32 (d, 1H), 4.57 (dd, 1H), 4.97 (dd, 1H), 6.90 (d, 1H). MS m/z 192 [M+H]⁺.

### Example 29c

### 2-(Methylamino)-1-(4-methylthiazol-2-yl)propan-1-ol

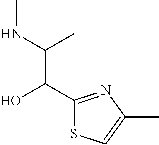

To 2-chloro-1-(4-methylthiazol-2-yl)propan-1-ol (Example 29b, 2.09 g, 10.88 mmol) was added methanamine (8M in EtOH, 13.60 ml, 108.83 mmol). The reaction was heated to 85° C. for 40 min in a microwave reactor. The solvents were evaporated giving the title compound (2.37 g, quantitative). ¹H NMR (500 MHz, DMSO-d₆) δ ppm 1.24 (d, 3H), 2.43 (d, 3H), 2.44 (s, 3H), 3.41 (s, 1H), 4.69 (d, 1H), 6.85 (d, 1H). MS m/z 187 [M+H]⁺.

### Example 29d

### 2-(((2,6-Dichloropyridin-3-yl)methyl)(methyl)amino)-1-(4-methylthiazol-2-yl)propan-1-ol

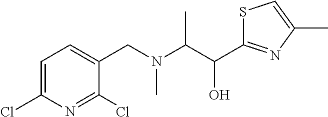

Triethylamine (0.825 mL, 5.92 mmol) was added to a stirred solution of 2-(methylamino)-1-(4-methylthiazol-2-yl)propan-1-ol (Example 29c, 1.102 g, 5.92 mmol) in MeCN (15 mL) at r.t. 3-(Bromomethyl)-2,6-dichloropyridine (CAS 58596-59-1, 1.425 g, 5.92 mmol) in MeCN (8 mL) was added slowly and the reaction mixture was stirred at r.t. for 1.5 h. The solvent was evaporated. The crude product was partitioned between water and DCM. The organic phase was dried over MgSO₄ and concentrated. The crude product was purified by silica flash chromatography using a gradient of MeOH (0 to 5%) in DCM giving the title compound (0.623 g, 30%). ¹H NMR (500 MHz, DMSO-d₆) δ ppm 1.05 (d, 3H), 1.53 (d, 1H), 2.19-2.23 (m, 3H), 2.28-2.31 (m, 3H), 2.98 (t, 1H), 3.57-3.67 (m, 1H), 3.78 (d, 1H), 4.47-4.56 (m, 1H), 4.73 (dd, 1H), 5.90 (d, 1H), 7.13 (d, 1H), 7.50 (d, 1H), 7.80 (d, 1H). MS m/z 346, 348, 350 [M+H]⁺.

### Example 29e

### 8-Chloro-3,4-dimethyl-2-(4-methylthiazol-2-yl)-2,3,4,5-tetrahydropyrido[3,2-f][1,4]oxazepine

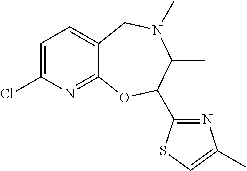

Potassium tert-pentoxide solution (1.7 M in toluene, 1.06 mL, 1.80 mmol) was added to a stirred solution of 2-(((2,6-dichloropyridin-3-yl)methyl)(methyl)amino)-1-(4-methylthiazol-2-yl)propan-1-ol (Example 29d, 0.623 g, 1.80 mmol) in toluene (15 mL) at r.t. and was stirred at r.t. overnight. The organic mixture was washed with water, dried over MgSO₄ and concentrated. The crude product was purified by silica flash chromatography using a gradient of MeOH (0 to 5%) in DCM giving the title compound (0.278 g, 50%). ¹H NMR (500 MHz, DMSO-d₆) δ ppm 1.01 (d, 3H), 2.36-2.37 (m, 3H), 2.42 (s, 3H), 3.47 (dd, 1H), 3.66 (d, 1H), 4.20 (d, 1H), 5.59 (d, 1H), 7.28 (s, 1H), 7.30 (d, 1H), 7.83 (d, 1H). MS m/z 310 [M+H]⁺.

### Example 29f

### N-(6-Methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine

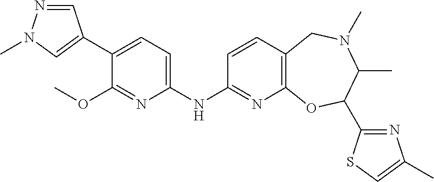

To 6-methoxy-5-(1-methyl-1H-pyrazol-4-yl)pyridin-2-ylamine (Example 2a, 0.086 g, 0.42 mmol) in DME (3 mL) were added 8-chloro-3,4-dimethyl-2-(4-methylthiazol-2-yl)-2,3,4,5-tetrahydropyrido[3,2-f][1,4]oxazepine (Example 29e, 0.130 g, 0.42 mmol), cesium carbonate (0.205 g, 0.63 mmol), 2-(dicyclohexylphosphino)biphenyl (0.015 g, 0.04 mmol) and palladium acetate (9.4 mg, 0.04 mmol). The reaction was heated to 110° C. for 60 min under argon atmosphere. The reaction mixture was filtered through Celite, washed with DCM and the solvents were evaporated. The crude product was purified by silica flash chromatography, 0-5% MeOH in DCM, yielding the title compound (0.147 g, 73%). MS m/z 478 [M+H]⁺.

### Example 30

### N-(6-Methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine, isomer 4

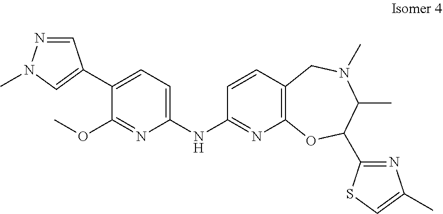

Separation of Example 29f according to Example 29 gave N-(6-methoxy-5-(1-methyl-1H-pyrazol-4-yl)pyridin-2-yl)-3,4-dimethyl-2-(4-methylthiazol-2-yl)-2,3,4,5-tetrahydropyrido[3,2-f][1,4]oxazepin-8-amine, isomer 4, the fourth isomer to elute (28.9 mg, 20%). ¹H NMR (500 MHz, DMSO-d₆) δ ppm 1.02 (d, 3H) 2.37 (s, 3H) 2.43 (s, 3H) 3.44-3.50 (m, 2H) 3.85 (s, 3H) 4.01 (s, 3H) 4.20 (d, 1H) 5.46 (d, 1H) 7.03 (d, 1H) 7.25 (s, 1H) 7.63 (d, 1H) 7.79-7.86 (m, 3H) 8.02 (s, 1H) 9.72 (s, 1H). MS m/z 478.1 [M+H]⁺.

### Example 31

### [8-(3-Fluoro-2-fluoromethyl-propyl)-6-methyl-5,6,7,8-tetrahydro-9-oxa-1,6-diaza-benzocyclohepten-2-yl]-[6-methoxy-5-(1-methyl-1H-pyrazol-4-yl)-pyridin-2-yl]-amine

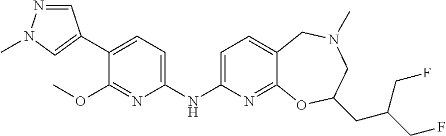

Pd(OAc)₂ (43 mg, 0.19 mmol) and Xantphos (225 mg, 0.38 mmol) were added to a degassed mixture of 2-chloro-8-(3-fluoro-2-fluoromethyl-propyl)-6-methyl-5,6,7,8-tetrahydro-9-oxa-1,6-diaza-benzocycloheptene (Example 31d, 190 mg, 0.65 mmol), 6-methoxy-3-(1-methyl-1H-pyrazol-4-yl)-pyridin-2-ylamine (Example 2a, 134 mg, 0.65 mmol) and cesium carbonate (275 mg, 0.84 mmol) in 1,4-dioxane (12 mL). The reaction mixture was purged with nitrogen for 15 min and then heated in a microwave reactor at 145° C. for 1.5 h. The mixture was cooled to r.t., diluted with ethyl acetate (40 mL) and filtered. The filtrate was concentrated in vacuo and the residue was purified by flash column chromatography using 5% MeOH in DCM to afford the title compound (100 mg, 33%). ¹H NMR (400 MHz, CDCl₃) δ ppm 1.82 (t, 1H) 2.43 (s, 3H) 2.63 (br. s., 1H) 2.80-2.97 (m, 2 H) 3.56 (d, 1H) 3.72 (d, 1H) 3.94 (s, 3H) 4.05 (s, 3H) 4.15 (br. s., 1H) 4.57-4.67 (m, 3H) 4.69-4.77 (m, 2H) 6.87 (d, 1H) 7.16 (s, 1H) 7.42-7.53 (m, 2H) 7.70 (d, 1H) 7.82 (s, 1H) 7.79 (s, 1H). ¹⁹F NMR (376 MHz, CDCl₃) δ ppm −231.12, −226.85. ESMS m/z 459.1 [M+H]⁺.

### Example 31a

### 5-Fluoro-4-fluoromethyl-pent-1-ene

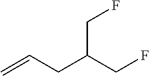

A solution of 2-allyl-malonic acid diethyl ester (12 mL, 0.06 mol) in dry THF (100 mL) was added slowly to a suspension of LiAlH₄ (6.8 g, 0.17 mol) in THF (400 mL) at −78° C. The reaction mixture was allowed to reach r.t., stirred o.n. and then poured slowly onto ice (500 g). The mixture was filtered through a pad of Celite and extracted with EtOAc (3×200 mL). The combined extracts were dried over MgSO₄ and concentrated in vacuo to afford 9 g of crude diol as a colourless oil. The crude diol (9.0 g, 0.077 mol) was dissolved in DCM (200 mL) and the solution was cooled to −78° C. DAST (28.5 mL, 0.23 mol) was then added and the reaction mixture was allowed to warm to r.t. and stirred o.n. The reaction was quenched with saturated NaHCO₃ solution and the phases were separated. The organic layer was dried over MgSO₄ and used directly in the next step without isolating the title compound. ¹H NMR (400 MHz, CDCl₃) δ ppm 2.01-2.10 (m, 1H) 2.12-2.21 (m, 2H) 4.34-4.45 (m, 1H) 4.48-4.56 (m, 1H) 5.01-5.17 (m, 2H) 5.70-5.85 (m, 1H).

### Example 31b

### 5-Fluoro-4-fluoromethyl-1-methylamino-pentan-2-ol

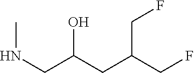

mCPBA (26.7 g, 173 mmol) was added to a solution of 5-fluoro-4-fluoromethyl-pent-1-ene (Example 31a, crude, 77.5 mmol) in DCM (200 mL) at 0° C. The reaction mixture was allowed to warm to r.t., stirred o.n. and then quenched with saturated KHSO₃ solution. The organic phase was separated, washed with 5% NaOH (20 mL) and dried over MgSO₄. DCM was removed by distillation at 50° C. to afford crude 2-(3-fluoro-2-fluoromethyl-propyl)-oxirane (7.6 g). The crude was dissolved in EtOH (20 mL) and methylamine (33 wt % in EtOH, 30 mL) was added. The reaction mixture was stirred for 6 h at r.t. and then concentrated in vacuo to afford 5-fluoro-4-fluoromethyl-1-methylamino-pentan-2-ol (8.5 g, crude) which was used in the next step without further purification. ¹H NMR (400 MHz, CDCl₃) δ ppm 1.77-1.87 (m, 1H) 2.22-2.39 (m, 1H) 2.50 (dd, 1H) 2.79-2.83 (m, 1H) 2.99-3.04 (m, 1H) 4.46 (d, 1H) 4.49 (d, 2H) 4.61 (d, 2H).

### Example 31c

### 1-[(2,6-Dichloro-pyridin-3-ylmethyl)-methyl-amino]-5-fluoro-4-fluoromethyl-pentan-2-ol

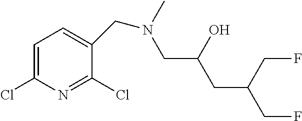

Preparation I. Cesium carbonate (3.5 g, 10.7 mmol) was added to a mixture of 5-fluoro-4-fluoromethyl-1-methylamino-pentan-2-ol (Example 31b, 1.5 g, 8.97 mmol) and 3-bromomethyl-2,6-dichloro-pyridine (CAS 58596-59-1, 2.1 g, 8.77 mmol) in DMF (30 mL) at 0° C. The reaction mixture was allowed to warm to r.t. and stirred o.n. The reaction mixture was then diluted with water (50 mL) and extracted with EtOAc (3×40 mL). The combined extracts were dried over MgSO₄, filtered and concentrated in vacuo. The residue was purified by flash column 1 chromatography using 30% EtOAc in DCM to give the title compound (900 mg, 31%). ¹H NMR (400 MHz, CDCl₃) δ ppm 1.38 (ddd, 1H) 1.51 (ddd, 1H) 2.27 (s, 3H) 2.40-2.48 (m, 3H) 3.58 (d, 1H) 3.72 (d, 1H) 3.84 (tt, 1H) 4.39-4.50 (m, 2H) 4.52-4.62 (m, 2H) 7.29 (d, 1H) 7.69 (d, 1H).

Preparation II. K₂CO₃ (7.0 g, 50.2 mmol) and 3-bromomethyl-2,6-dichloro-pyridine (CAS 58596-59-1, 12.1 g, 50.2 mmol) was added to a solution of 5-fluoro-4-fluoromethyl-1-methylamino-pentan-2-ol (Example 31b, 12.1 g, 50.2 mmol) in methanol (100 mL). The mixture was stirred at r.t. o.n. and concentrated in vacuo. The residue was partitioned with water and EtOAc. The two phases were separated. The aqueous phase was extracted with EtOAc (×2). The organic phases were combined, dried over MgSO₄ and concentrated. The residue was purified by chromatography on silica gel with a mixture of EtOAc and heptane (0%-50%) to an oil. The oil was dissolved in MTBE (100 mL) and acidified with 1N HCl (90 mL). The aqueous phase was separated and basified to pH 9 with 25% NaOH (12 mL). The mixture was then extracted with MTBE, dried over MgSO₄ and concentrated to yield the title compound as an oil (9.4 g, 57%). ¹H NMR (300 MHz, CDCl₃): ppm 1.31-1.43 (m, 1H), 1.50 (ddd, 1H), 2.27 (s, 3H), 2.35-2.50 (m, 2H), 3.21-3.30 (m, 1H), 3.58 (d, 1H), 3.72 (d, 1H), 3.78-3.90 (m, 1H), 4.36-4.50 (m, 2H), 4.51-5.66 (m, 2H), 7.28 (d, 1H), 7.69 (d, 1H).

### Example 31d

### 2-Chloro-8-(3-fluoro-2-fluoromethyl-propyl)-6-methyl-5,6,7,8-tetrahydro-9-oxa-1,6-diaza-benzocycloheptene

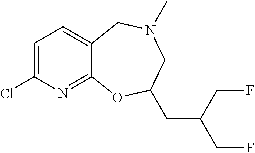

Sodium hydride (57% in mineral oil, 67 mg, 1.6 mmol) was added to a solution of 1-[(2,6-dichloro-pyridin-3-ylmethyl)-methyl-amino]-5-fluoro-4-fluoromethyl-pentan-2-ol (Example 31c, Preparation I; 400 mg, 1.22 mmol) in dry THF (30 mL) at 0° C. The reaction mixture was heated at 40° C. for 3 h, then cooled to r.t., quenched with saturated NH₄Cl solution and extracted with EtOAc (3×30 mL). The combined extracts were dried over MgSO₄, filtered and concentrated in vacuo. The residue was purified by flash column chromatography using 80% EtOAc in DCM to afford the title compound (305 mg, 85%). ¹H NMR (400 MHz, CDCl₃) δ ppm 1.60 (ddd, 1H) 1.76-1.89 (m, 1H) 2.42 (s, 3 H) 2.50-2.71 (m, 1H) 2.81-2.97 (m, 2H) 3.62 (d, 1H) 3.72 (d, 1H) 4.13-4.22 (m, 1H) 4.45-4.57 (m, 2H) 4.58-4.69 (m, 2H) 7.04 (d, 1H) 7.46 (d, 1H).

### Example 32

### [8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine

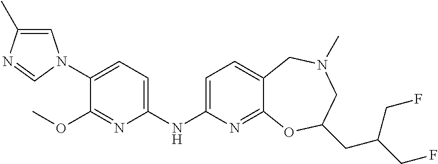

Preparation I. Pd(OAc)₂ (47 mg, 0.20 mmol) and Xantphos (239 mg, 0.41 mmol) were added to a degassed mixture of 2-chloro-8-(3-fluoro-2-fluoromethyl-propyl)-6-methyl-5,6,7,8-tetrahydro-9-oxa-1,6-diaza-benzocycloheptene (Example 31d, 201 mg, 0.69 mmol), 6-methoxy-5-(4-methyl-imidazol-1-yl)-pyridin-2-ylamine (Example 6a, 141 mg, 0.69 mmol) and cesium carbonate (337 mg, 1.03 mmol) in 1,4-dioxane (12 mL). The reaction mixture was purged with nitrogen for 15 min and then heated in a microwave reactor at 145° C. for 1.5 h. The reaction mixture was cooled to r.t., diluted with ethyl acetate (40 mL) and filtered. The filtrate was concentrated in vacuo and the residue was purified by flash column chromatography using 5% MeOH in DCM to afford the title compound (144 mg, 45%). ¹H NMR (400 MHz, CDCl₃) δ ppm 1.78-1.88 (m, 1H) 2.29 (s, 3H) 2.43 (s, 3H) 2.52-2.71 (m, 2H) 2.80-2.96 (m, 2H) 3.57 (d, 1H) 3.72 (d, 1H) 3.98 (s, 3H) 4.11-4.18 (m, 1H) 4.49-4.78 (m, 5H) 6.87 (s, 1H) 7.05 (d, 1H) 7.17 (s, 1H) 7.32 (d, 1H) 7.46 (dd, 2 H) 7.63 (s, 1H). ¹⁹F NMR (376 MHz, CDCl₃) δ ppm −231.06, −226.98. ESMS m/z 459.2 [M+H]⁺.

Preparation II. A mixture of 6-methoxy-5-(4-methyl-imidazol-1-yl)-pyridin-2-ylamine (Example 6a, 0.5 g, 2.45 mmol), 2-chloro-8-(3-fluoro-2-fluoromethyl-propyl)-6-methyl-5,6,7,8-tetrahydro-9-oxa-1,6-diaza-benzocycloheptene (Example 31d, 0.705 g, 2.42 mmol), (1,1′-bis(diphenylphosphino)ferrocene)-dichloropalladium(II) (0.020 g, 0.02 mmol), 9,9-dimethyl-4,5-bis(diphenylphosphino)xanthene (0.014 g, 0.02 mmol) and sodium tert-pentoxide (0.400 g, 3.64 mmol) in dry degassed toluene (20 mL) was heated at 120° C. for 18 h. The reaction mixture was cooled to r.t., and concentrated in vacuo. The residue was treated with water (60 mL) and extracted with DCM (60 mL). The organic phase was washed with brine, dried over anhydrous sodium sulfate, filtered and concentrated. The residue was purified by flash column chromatography using 0-100% DCM:MeOH:NH₃ (900:90:10) as an eluent to afford the title compound (0.620 g, 55.8%). ESMS m/z 459 [M+H]⁺.

### Example 33

### N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine; isomer 1

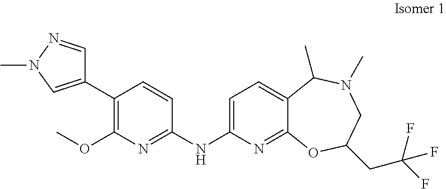

The four isomers of N-(6-methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine (Example 33e, 305 mg, 0.66 mmol) were separated using SFC [Column: Chiralpak OJ-H (21.2*250 mm), Mobile phase: (15% EtOH/IPA (1:1)+0.1% DEA; 85% CO₂), Flow: 50 mL/min] to afford N-(6-methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine, isomer 1, the first isomer to elute (51 mg, 17%). ¹H NMR (500 MHz, DMSO-d₆) δ ppm 1.34 (d, 3H) 2.33 (s, 3H) 2.52-2.80 (m, 3H) 3.72 (q, 1H) 3.85 (s, 3H) 3.99 (s, 3H) 4.24-4.36 (m, 1H) 7.22 (d, 1H) 7.52 (d, 1H) 7.60 (d, 1H) 7.82 (d, 1H) 7.85 (s, 1H) 8.02 (s, 1H) 9.53 (s, 1H). MS m/z 463.1 [M+H]⁺.

### Example 33a

### 1-Amino-4,4,4-trifluoro-butan-2-ol

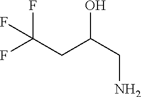

Methanolic ammonia (70 mL) was added to a solution of 2-(2,2,2-trifluoro-ethyl)-oxirane (5.0 g, 39.7 mmol) in MeOH (150 mL) at r.t. The reaction mixture was heated in a pressure vessel at 60° C. for 3 h. The reaction mixture was then cooled to r.t. and concentrated under reduced pressure to afford the title compound (4.2 g, 74%) which was used in the next step without further purification. ¹H NMR (400 MHz, CDCl₃) δ ppm 2.12-2.27 (m, 1H) 2.27-2.42 (m, 1H) 2.57-2.69 (m, 1H) 2.92 (dd, 1H) 3.83-3.97 (m, 1H).

### Example 33b

### 1-[1-(2,6-Dichloro-pyridin-3-yl)-ethylamino]-4,4,4-trifluoro-butan-2-ol

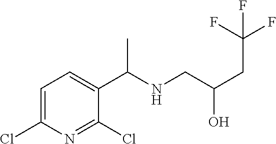

1-Amino-4,4,4-trifluoro-butan-2-ol (Example 33a, 1.98 g, 13.81 mmol) was added to a mixture of cesium carbonate (8.2 g, 25.1 mmol) and 3-(1-bromo-ethyl)-2,6-dichloro-pyridine (Example 1b, 3.2 g, 12.55 mmol) in anhydrous DMF (100 mL) at r.t. under a nitrogen atmosphere. The reaction mixture was stirred o.n. then filtered and concentrated under reduced pressure. The residue was purified by flash column chromatography using a gradient of 0 to 5% MeOH in DCM to afford the title compound (1.5 g, 38%). ¹H NMR (400 MHz, CDCl₃) δ ppm 1.36 (d, 3H) 2.11-2.26 (m, 1H) 2.27-2.41 (m, 1H) 2.50-2.62 (m, 1H) 2.73 (dd, 1H) 3.88-4.09 (m, 1H) 4.15-4.30 (m, 1H) 7.31 (d, 1H) 7.80 (dd, 1H). ESMS m/z 318.8 [M+H]⁺.

### Example 33c

### 2-Chloro-5-methyl-8-(2,2,2-trifluoro-ethyl)-5,6,7,8-tetrahydro-9-oxa-1,6-diaza-benzocycloheptene

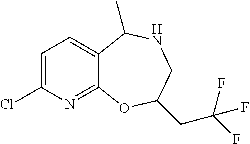

Sodium hydride (95% powder, 0.144 g, 5.68 mmol) was added to a solution of 1-[1-(2,6-dichloro-pyridin-3-yl)-ethylamino]-4,4,4-trifluoro-butan-2-ol (Example 33b, 1.5 g, 4.73 mmol) in anhydrous THF (100 mL) at −40° C. The reaction mixture was allowed to warm to r.t. and was stirred o.n. The reaction was quenched with a saturated solution of NH₄Cl (5.0 mL) and concentrated under reduced pressure. The residue was taken in ethyl acetate (50 mL) and the organic phase was washed with water and brine, dried over Na₂SO₄, and concentrated under reduced pressure. The residue was purified by flash column chromatography using a gradient of 0 to 5% MeOH in DCM to afford the title compound (0.9 g, 68%). ¹H NMR (400 MHz, CDCl₃) δ ppm 1.40-1.58 (m, 3H) 2.32-2.55 (m, 1H) 2.66-2.88 (m, 1H) 3.10-3.26 (m, 1H) 3.27-3.44 (m, 1H) 3.92-4.07 (m, 1H) 7.03-7.16 (m, 1H) 7.41-7.58 (m, 1H).

### Example 33d

### 2-Chloro-5,6-dimethyl-8-(2,2,2-trifluoro-ethyl)-5,6,7,8-tetrahydro-9-oxa-1,6-diaza-benzocycloheptene

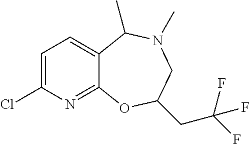

A mixture of 2-chloro-5-methyl-8-(2,2,2-trifluoro-ethyl)-5,6,7,8-tetrahydro-9-oxa-1,6-diaza-benzocycloheptene (Example 33c, 0.9 g, 3.21 mmol) and paraformaldehyde (4.0 g) in MeOH (100 mL) was stirred at r.t. for 1 h under a nitrogen atmosphere. Sodium triacetoxy borohydride (3.4 g) was added and the resulting slurry was stirred at r.t. for 24 h. The precipitated solid was removed by filtration and the filtrate was concentrated under reduced pressure. The residue was purified by flash column chromatography using a gradient of 0 to 5% MeOH in DCM to afford the title compound (0.81 g, 85%). ¹H NMR (400 MHz, CDCl₃) δ ppm 1.46 (t, 3H) 2.21 (s, 2H) 2.29-2.51 (m, 4H) 2.66-2.92 (m, 2H) 3.10-3.31 (m, 1H) 3.35-3.58 (m, 1H) 3.79 (d, 1H) 4.21 (d, 1H) 4.33-4.46 (m, 1H) 4.85-4.96 (m, 3H) 7.10 (dd, 1H) 7.47 (dd, 1H). ESMS m/z 295.0 [M+H]⁺.

### Example 33e

### N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine

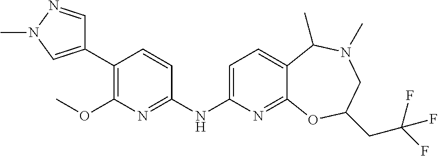

Palladium acetate (0.023 g, 5 mol %), Xantphos (0.118 g, 30 mol %) and cesium carbonate (0.33 g, 1.02 mmol) were added to a degassed mixture of 2-chloro-5,6-dimethyl-8-(2,2,2-trifluoro-ethyl)-5,6,7,8-tetrahydro-9-oxa-1,6-diaza-benzocycloheptene (Example 33d, 0.2 g, 0.68 mmol), 6-methoxy-5-(1-methyl-1H-pyrazol-4-yl)-pyridin-2-ylamine (Example 2a, 0.153 g, 0.68 mmol) in anhydrous 1,4-dioxane (20 mL). The reaction mixtures was purged with nitrogen for 15 min and then heated in a microwave reactor at 145° C. for 1 h. The reaction mixture was cooled to r.t., diluted with DCM (20 mL) and filtered through a small pad of Celite. The filtrate was concentrated under reduced pressure and the residue was purified by flash column chromatography using a gradient of 0 to 10% MeOH in DCM to afford the title compound (320 mg, quantitative). ¹H NMR (400 MHz, CDCl₃) δ ppm 1.48 (d, 3H) 1.58 (br. s., 1H) 2.23 (s, 1H) 2.25-2.42 (m, 1H) 2.44-2.50 (m, 1H) 2.62-2.89 (m, 1H) 3.00-3.35 (m, 1H) 3.45-3.82 (m, 1H) 3.94 (s, 3H) 4.06 (d, 3H) 4.13-4.55 (m, 1H) 6.82 (dd, 1H) 7.12 (s, 1H) 7.38-7.53 (m, 1H) 7.51-7.67 (m, 1H) 7.68-7.76 (m, 1H) 7.80 (d, 2H). ESMS m/z 463.1 [M+H]⁺.

### Example 34

### N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine; isomer 2

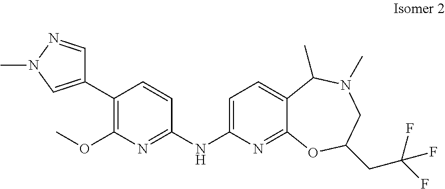

Separation as in Example 33 to give N-(6-methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine, isomer 2 the second isomer to elute (62 mg, 20%). ¹H NMR (500 MHz, DMSO-d₆) δ ppm 1.38 (d, 3H) 2.09 (s, 3H) 2.52-2.72 (m, 2H) 2.98 (dd, 1H) 3.13 (dd, 1H) 3.85 (s, 3H) 4.00 (s, 3H) 4.11 (q, 1H) 4.24-4.36 (m, 1H) 7.22 (d, 1H) 7.54 (d, 1H) 7.67 (d, 1H) 7.83 (d, 1H) 7.85 (s, 1H) 8.02 (s, 1H) 9.55 (s, 1H). MS m/z 463.1 [M+H]⁺.

### Example 35

### N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine; isomer 3

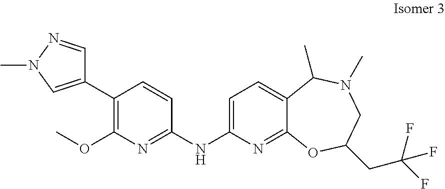

Separation as in Example 33 to give N-(6-methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine, isomer 3 the third isomer to elute (59 mg, 19%). ¹H NMR (500 MHz, DMSO-d₆) δ ppm 1.38 (d, 3H) 2.09 (s, 3 H) 2.52-2.70 (m, 2H) 2.98 (dd, 1H) 3.14 (dd, 1H) 3.85 (s, 3H) 4.00 (s, 3H) 4.12 (q, 1H) 4.24-4.35 (m, 1H) 7.22 (d, 1H) 7.54 (d, 1H) 7.67 (d, 1H) 7.83 (d, 1H) 7.85 (d, 1H) 8.02 (s, 1H) 9.55 (s, 1H). MS m/z 463.2 [M+H]⁺.

### Example 36

### N-(6-Methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine; isomer 4

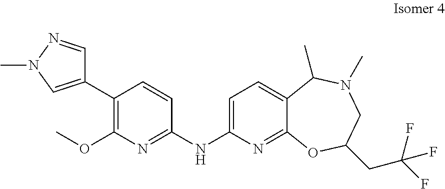

Separation as in Example 33 to give N-(6-methoxy-5-(1-methyl-1H-pyrazol-4-yl)pyridin-2-yl)-4,5-dimethyl-2-(2,2,2-trifluoroethyl)-2,3,4,5-tetrahydropyrido[3,2-f][1,4]oxazepin-8-amine, isomer 4 the fourth isomer to elute (46 mg, 15%). ¹H NMR (500 MHz, DMSO-d₆) δ ppm 1.38 (d, 3H) 2.09 (s, 3 H) 2.52-2.70 (m, 2H) 2.98 (dd, 1H) 3.14 (dd, 1H) 3.85 (s, 3H) 4.00 (s, 3H) 4.12 (q, 1H) 4.24-4.35 (m, 1H) 7.22 (d, 1H) 7.54 (d, 1H) 7.67 (d, 1H) 7.83 (d, 1H) 7.85 (d, 1H) 8.02 (s, 1H) 9.55 (s, 1H). MS m/z 463.2 [M+H]⁺.

### Example 37

### 2-(3,3-Difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine; isomer 1

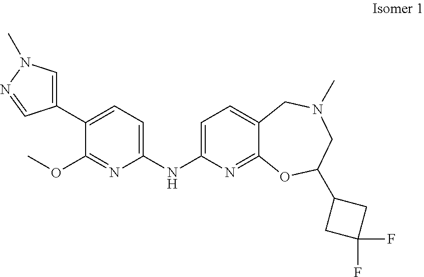

2-(3,3-Difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine (Example 37a, 0.086 g, 0.19 mmol) was subjected to chiral separation using SFC chromatography [Column: Chiralpak AD-H (21.2*250 mm) Mobile phase: 25% EtOH+0.1% DEA: 80% CO₂; Flow 50 mL/min]. The solvent was evaporated and the residue was partitioned between water and DCM. The organic layer was dried over Na₂SO₄, filtered and concentrated giving 2-(3,3-difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine, isomer 1, the first isomer to elute (0.025 g, 29%). ¹H NMR (500 MHz, DMSO-d₆) δ ppm 2.29 (s, 3H), 2.31-2.38 (m, 1H), 2.58-2.62 (m, 2H), 2.65-2.72 (m, 3H), 2.85 (d, 1H), 3.51-3.61 (m, 2H), 3.85 (s, 3H), 3.94-4.04 (m, 4H), 7.05 (d, 1H), 7.55 (d, 1H), 7.69 (d, 1H), 7.83-7.86 (m, 2H), 8.02 (s, 1H), 9.61 (s, 1H). MS m/z 457 [M+H]⁺.

### Example 37a

### 2-(3,3-Difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine

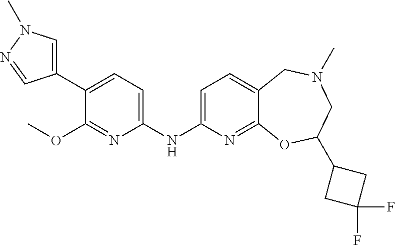

Palladium acetate (19 mg, 0.083 mmol) and Xantphos (96 mg, 0.16 mmol) were added to a degassed mixture of 2-chloro-8-(3,3-difluoro-cyclobutyl)-6-methyl-5,6,7,8-tetrahydro-9-oxa-1,6-diaza-benzocycloheptene (Example 37l, 160 mg, 0.55 mmol), 6-methoxy-5-(1-methyl-1H-pyrazol-4-yl)-pyridin-2-ylamine (Example 2a, 113 mg, 0.55 mmol) and cesium carbonate (235 mg, 0.72 mmol) in dioxane (15 mL). The reaction mixture was purged with nitrogen for 20 min and heated in a microwave reactor at 145° C. for 1 h. The reaction mixture was cooled to r.t., diluted with DCM, and filtered through a pad of Celite. The filtrate was concentrated in vacuo and the residue was purified by flash column chromatography using a gradient of 2 to 5% MeOH in DCM to afford the title compound (140 mg, 56%). ¹H NMR (400 MHz, CDCl₃) δ ppm 2.36 (m, 1H) 2.42 (s, 3H) 2.75 (m, 6H) 3.57 (d, 1H) 3.67 (d, 1H) 3.94 (s, 3H) 3.98 (t, 1H) 4.05 (s, 3H) 6.82 (d, 1H) 7.10 (s, 1H) 7.43 (d, 1H) 7.52 (d, 1H) 7.70 (d, 1H) 7.78 (s, 1H) 7.81 (s, 1H). ESMS m/z 457.17 [M+H]⁺.

### Example 37b

### 3-Oxo-cyclobutanecarbonitrile

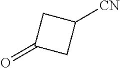

Sodium metaperiodate (284 g, 1.32 mol) was added in small portions over a period of 30-45 minutes to mixture of 3-methylene cyclobutanecarbonitrile (30 g, 370 mol) and ruthenium trichloride monohydrate (2.2 mol %, 1.5 g, 7.23 mmol) in a mixture of DCM-MeCN-water (645 mL:645 mL:945 mL) at 5° C. The reaction mixture was vigorously stirred at r.t. for 28 h. The organic phase was then separated and the aqueous phase was extracted with DCM (3×1500 mL). The combined organic extracts were filtered through a small pad of flash silica gel, and the pad was washed with 2000 mL of DCM. The filtrate was dried over MgSO₄, filtered and concentrated under reduced pressure to afford 3-oxo-cyclobutanecarbonitrile (28 g, 100%). ¹H NMR (400 MHz, CDCl₃) δ ppm 3.27 (quin, 1H) 3.57 (d, 4H).

### Example 37c

### 3,3-Difluoro-cyclobutanecarbonitrile

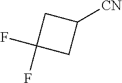

Neat DAST (94.9 g, 0.59 mol) was added drop wise to a solution of 3-oxo-cyclobutanecarbonitrile (Example 37b, 28 g, 294 mol) in DCM (1000 mL) at −10° C. The reaction was allowed to warm to r.t. and was stirred for 24 h. The reaction mixture was then poured slowly into an ice-cold saturated NaHCO₃ solution. The organic layer was separated and the aqueous phase was re-extracted with DCM (2×500 mL). The combined organic extracts were dried over MgSO₄, filtered and carefully concentrated under reduced pressure using low temperature water bath (<10° C.) to afford 3,3-difluoro-cyclobutanecarbonitrile (40 g, 82%). ¹H NMR (400 MHz, CDCl₃) δ ppm 2.92-3.05 (m, 5 H).

### Example 37d

### 3,3-Difluoro-cyclobutanecarboxylic acid

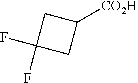

Sodium hydroxide pellets (16.39 g, 0.41 mol) were added to a solution of 3,3-difluoro-cyclobutanecarbonitrile (Example 37c, 40 g, 0.34 mol) in MeOH (500 mL) at r.t. followed by addition of water (150 mL). The resulting mixture was stirred at r.t. for 72 h and then heated at 60° C. for 3 h. The reaction mixture was cooled to r.t. and concentrated under reduced pressure. The residue was taken into water (50 mL) and washed with ethyl acetate (2×50 mL) to remove small amount of the amide by-product. The aqueous phase was first acidified (pH=1) using 2 M HCl and then extracted with DCM (3×50 mL) and diethyl ether (2×50 mL). The combined organic extracts were dried over MgSO₄ and concentrated under reduced pressure. The residue was dissolved in a mixture of diethyl ether and pentane (2:1, 500 mL) and passed through a pad of silica gel. The filtrate was concentrated under reduced pressure to obtain 3,3-difluoro-cyclobutanecarboxylic acid (37 g, quantitative). ¹H NMR (400 MHz, CDCl₃) δ ppm 2.78-2.94 (m, 4H) 2.95-3.11 (m, 1H).

### Example 37e

### 3,3-Difluoro-cyclobutanecarboxylic acid ethyl ester

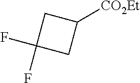

Ethyl iodide (51.6 g, 26.6 mL, 330 mmol) was added to a mixture of 3-difluoro-cyclobutanecarboxylic acid (Example 37d, 30 g, 220 mmol) and cesium carbonate (71.8 g, 220 mmol) in anhydrous DMF (150 mL) at r.t. under a nitrogen atmosphere. The reaction was stirred at r.t. o.n. and the precipitated solid was removed by filtration. The filtrate was diluted with water (200 mL) and extracted with ethyl acetate (3×100 mL). The combined organic extracts were washed with brine, dried over Na₂SO₄ and concentrated under partial vacuum at 0° C. to afford 3,3-difluoro-1 cyclobutanecarboxylic acid ethyl ester (36.4 g, quantitative). ¹H NMR (400 MHz, CDCl₃) δ ppm 1.27-1.33 (m, 3H) 2.73-2.89 (m, 4H) 2.89-3.01 (m, 1H) 4.08-4.15 (m, 2H).

### Example 37f

### (3,3-Difluoro-cyclobutyl)-methanol

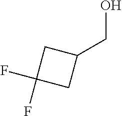

Lithium aluminum hydride solution (2.0 M in THF, 110.4 mL, 221.7 mmol) was added drop wise to a solution of 3,3-difluoro-cyclobutanecarboxylic acid ethyl ester (Example 37e, 36.4 g, 221.75 mmol) in anhydrous THF (1000 mL) at −30° C. under a nitrogen atmosphere. The reaction mixture was allowed to warm to r.t. and stirred o.n. The reaction was carefully quenched with saturated ammonium chloride solution at 0° C. The precipitate was removed by filtration and the filtrate was concentrated under partial pressure at 0° C. to afford (3,3-difluoro-cyclobutyl)-methanol (11 g, 41%). ¹H NMR (400 MHz, CDCl₃) δ ppm 1.64 (d, 2H) 1.86 (ddd, 1H) 2.26-2.44 (m, 2H) 2.55-2.73 (m, 1H) 3.69 (d, 1H) 3.72-3.80 (m, 1H).

### Example 37g

### 3,3-Difluoro-cyclobutanecarbaldehyde

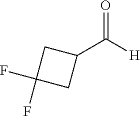

A solution of (3,3-difluoro-cyclobutyl)-methanol (Example 37f, 3.0 g, 25 mmol) in DCM (10 mL) was added to a suspension of pyridinium chlorochromate (8.6 g, 40 mmol) in DCM (50 mL) at r.t. The reaction mixture was stirred at r.t. for 1 h and then diluted with diethyl ether (100 mL). The reaction mixture was filtered through a plug of silica gel (250-400 mesh) and Celite. The filtrate was concentrated at 0° C. using partial pressure to obtain 3,3-difluoro-cyclobutanecarbaldehyde. This procedure was repeated using the same amounts and the two batches were combined to give a total yield of 3.2 g, (54%). ¹H NMR (400 MHz, CDCl₃) δ ppm 2.25-2.43 (m, 1H) 2.43-2.54 (m, 1H) 2.56-2.71 (m, 1H) 2.71-2.87 (m, 1H) 2.97-3.11 (m, 1H) 9.78 (s, 1H).

### Example 37h

### 1-(3,3-Difluoro-cyclobutyl)-2-nitro-ethanol

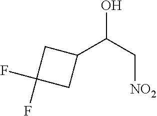

Diisopropylethyl amine (4.31 g, 33.33 mmol) was added to a mixture of 3,3-difluoro-cyclobutanecarbaldehyde (Example 37g, 3.0 g, 16.67 mmol) and nitromethane (5.0 mL, excess) in anhydrous DCM (60 mL) at 0° C. The reaction mixture was allowed to slowly reach r.t. over 1 h and then stirred for 72 h. The volatiles were removed under reduced pressure and the residue was purified by flash column chromatography using a gradient of 0 to 10% ethyl acetate in hexane to afford the title compound (2.85 g, 63%). ¹H NMR (400 MHz, CDCl₃) δ ppm 1.59 (br. s., 1H) 2.56-2.74 (m, 4H) 3.70 (d, 1H) 3.73-3.79 (m, 1H) 4.29-4.36 (m, 2H). ESMS m/z 183.0 [M+H]⁺.

### Example 37i

### 2-Amino-1-(3,3-difluoro-cyclobutyl)-ethanol

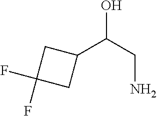

Pd/C (10 wt % wet, 0.3 g) was added to a solution of 1-(3,3-difluoro-cyclobutyl)-2-nitro-ethanol (Example 37h, 2.85 g, 15.73 mmol) in MeOH (50 mL). The mixture was shaken under a hydrogen atmosphere (45 psi) at r.t. for 24 h. The mixture was filtered through a small plug of Celite and the filtrate was concentrated under reduced pressure to afford 2-amino-1-(3,3-difluoro-cyclobutyl)-ethanol (2.4 g, 100%) which was used in the next step without further purification. ¹H NMR (400 MHz, CDCl₃) δ ppm 2.14 (br. s., 1H) 2.29-2.45 (m, 1H) 2.46 (m, 1H) 2.46-2.73 (m, 3H) 2.87 (dd, 1H) 2.95 (br. s., 2H) 3.53-3.63 (m, 1H) 3.69 (m, 1H). ESMS m/z 152.0 [M+H]⁺.

### Example 37j

### 2-[(2,6-Dichloro-pyridin-3-ylmethyl)-amino]-1-(3,3-difluoro-cyclobutyl)-ethanol

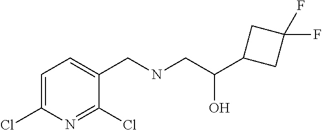

3-Bromomethyl-2,6-dichloro-pyridine (CAS 58596-59-1, 845 mg, 3.52 mmol) was added to a mixture of 2-amino-1-(3,3-difluoro-cyclobutyl)-ethanol (Example 37l, 638 mg, 4.22 mmol) and cesium carbonate (2.3 g, 7.04 mmol) in DMF (20 mL). The reaction mixture was stirred at r.t. o.n., then diluted with DCM (200 mL), filtered through a pad of Celite and concentrated in vacuo. The residue was purified by flash column chromatography using a gradient of 25 to 50% ethyl acetate in hexane to afford the title compound (376 mg, 34%). ¹H NMR (400 MHz, CDCl₃) δ 2.51 (m, 4H) 3.12 (dd, 1H) 3.65 (t, 1H) 4.54 (m, 3H) 7.32 (d, 1H) 7.74 (d, 1H).

### Example 37k

### 2-Chloro-8-(3,3-difluoro-cyclobutyl)-5,6,7,8-tetrahydro-9-oxa-1,6-diaza-benzocycloheptene

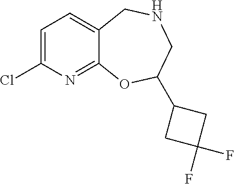

Sodium hydride (138 mg, 95%, 5.76 mmol) was added to a solution of 2-[(2,6-dichloro-pyridin-3-ylmethyl)-amino]-1-(3,3-difluoro-cyclobutyl)-ethanol (Example 37j, 893 mg, 2.88 mmol) in THF (40 mL) at 0° C. The reaction mixture was allowed to warm to r.t. and stirred for 2 h. An additional portion of sodium hydride (70 mg, 2.88 mmol) was added and the reaction mixture was heated at 40° C. o.n. The reaction mixture was cooled to r.t., quenched with saturated ammonium chloride and extracted with ethyl acetate (2×100 mL). The combined organic extracts were dried over magnesium sulfate, filtered and concentrated in vacuo. The residue was purified by flash column chromatography using a gradient of 2 to 5% MeOH in DCM to afford the title compound (170 mg, 22%). ¹H NMR (400 MHz, CDCl₃) δ 2.38 (m, 2H) 2.66 (m, 4H) 2.96 (m, 1H) 3.20 (dd, 1H) 3.90 (m, 3H) 7.03 (d, 1H) 7.43 (d, 1H).

### Example 37l

### 2-Chloro-8-(3,3-difluoro-cyclobutyl)-6-methyl-5,6,7,8-tetrahydro-9-oxa-1,6-diaza-benzocycloheptene

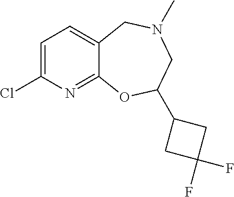

A mixture of 2-chloro-8-(3,3-difluoro-cyclobutyl)-5,6,7,8-tetrahydro-9-oxa-1,6-diaza-benzocycloheptene (Example 37k, 170 mg, 0.62 mmol) and paraformaldehyde (850 mg, 28 mmol) in MeOH (30 mL) was stirred for 6 h at r.t. Sodium triacetoxyborohydride (800 mg, 3.77 mmol) was added and the reaction mixture stirred at r.t. o.n. The mixture was filtered through a pad of Celite, filtered and concentrated in vacuo. The residue was partitioned between saturated sodium bicarbonate solution and DCM. The phases were separated and the aqueous layer was re-extracted with DCM. The combined organic extracts were dried over magnesium sulfate, filtered and concentrated in vacuo to afford the title compound (163 mg, 91%). ¹H NMR (400 MHz, CDCl₃) δ 2.31 (m, 3H) 2.42 (s, 3H) 2.73 (m, 7H) 2.88 (m, 1H) 3.62 (d, 1H) 3.69 (d, 1H) 4.00 (t, 1H) 7.04 (d, 1H) 7.45 (d, 1H).

### Example 38

### 2-(3,3-Difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine; isomer 2

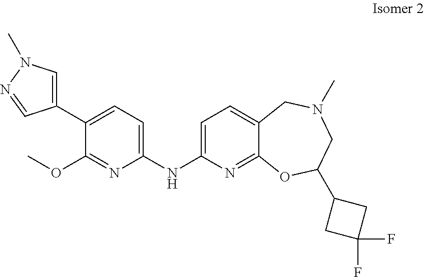

Separation as in Example 37 gave 2-(3,3-difluorocyclobutyl)-N-(6-methoxy-5-(1-methyl-1H-pyrazol-4-yl)pyridin-2-yl)-4-methyl-2,3,4,5-tetrahydropyrido[3,2-f][1,4]oxazepin-8-amine, isomer 2, the second isomer to elute (0.030 g, 35%). ¹H NMR (400 MHz, CDCl₃) δ ppm 2.38 (s, 3H) 2.50 (s, 3H) 3.09 (dd, 1H) 3.41 (d, 1H) 3.69 (d, 1H) 3.93 (s, 3H) 3.96 (d, 1H) 4.06 (s, 3H) 5.15-5.23 (m, 1H) 6.53 (d, 1H) 7.02 (d, 1H) 7.37 (s, 1H) 7.52 (d, 1H) 7.57 (s, 1H) 7.64 (d, 1H) 7.75-7.83 (m, 3H) 8.40 (d, 1H). MS m/z 457 [M+H]⁺.

### Example 39

### N-(6-Methoxy-5-(1-methyl-1H-pyrazol-4-yl)pyridin-2-yl)-4-methyl-2-(5-methylthiazol-2-yl)-2,3,4,5-tetrahydropyrido[3,2-f][1,4]oxazepin-8-amine

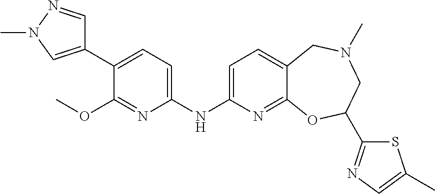

8-Chloro-4-methyl-2-(5-methylthiazol-2-yl)-2,3,4,5-tetrahydropyrido[3,2-f][1,4]oxazepine (Example 39e, 139 mg, 0.47 mmol), 6-methoxy-5-(1-methyl-1H-pyrazol-4-yl)pyridin-2-ylamine (Example 2a, 106 mg, 0.52 mmol), 2-(dicyclohexylphosphino)biphenyl (16.47 mg, 0.05 mmol), Pd(OAc)₂ (10.55 mg, 0.05 mmol) and cesium carbonate (383 mg, 1.17 mmol) were weighed into a microwave vial, the vial was capped and DME (4 mL) was added. The vial was flushed with argon and heated to 100° C. in a microwave reactor for 1 h. The reaction mixture was diluted with DCM, filtered and the solvents were evaporated. The residue was purified by column chromatography on silica using gradient elution with MeOH in DCM (0-6%) to give the title compound (121 mg, 75%). ¹H NMR (500 MHz, CDCl₃) δ ppm 2.49-2.52 (m, 3H), 2.53 (s, 3H), 3.20 (m, 1H), 3.57-3.72 (m, 2H), 3.91-4.00 (m, 4H), 4.07 (s, 3H), 5.35 (d, 1H), 6.72 (d, 1H), 7.23 (s, 1H), 7.43 (m, 1H), 7.53 (d, 1H), 7.71 (app dd, 2H), 7.80 (s, 1H), 7.82 (s, 1H). MS m/z 464 [M+H]⁺.

### Example 39a

### 2-Chloro-1-(5-methylthiazol-2-yl)ethanone

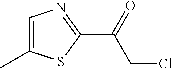

Isopropylmagnesium bromide (CAS 920-39-8, 1 M in THF, 12.1 mL, 12.1 mmol) was added dropwise to a solution of 2-bromo-5-methylthiazole (CAS 41731-23-1, 2.056 g, 11.55 mmol) in THF (20 mL) at 0° C. and the resulting solution was stirred for 15 min at 0° C. A solution of 2-chloro-1-morpholinoethanone (CAS 1440-61-5, 2.078 g, 12.70 mmol) in THF (5 mL) was added dropwise and the mixture was stirred at 0° C. for 45 min and then at room temperature for 1.5 h. The reaction was quenched by the addition of sat. aq. NH₄Cl and the mixture was diluted with diethyl ether. The phases were separated and the aqueous phase was extracted with diethyl ether. The combined organic layers were washed with citric acid, water and sat. aq. NaHCO₃, dried over MgSO₄ and concentrated to give 2-chloro-1-(5-methylthiazol-2-yl)ethanone (1.48 g, 73%). MS m/z 176, 178 [M+H]⁺.

### Example 39b

### 2-Chloro-1-(5-methylthiazol-2-yl)ethanol

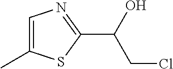

Sodium borohydride (0.349 g, 9.23 mmol) was added to a solution of 2-chloro-1-(5-methylthiazol-2-yl)ethanone (Example 39a, 1.47 g, 8.39 mmol) in MeOH (10 mL) and THF (5 mL) while keeping the temperature below −20° C. The temperature was kept at or below −20° C. for 2 h and then slowly allowed to rise to 15° C. and stirred o.n. The solvents were evaporated and the residue was partitioned between ethyl acetate and sat. aq. NaHCO₃. The organic phase was dried, filtered and the solvent evaporated to give the title compound (1.25 g, 84%). MS m/z 178, 180 [M+H]⁺.

### Example 39

### 2-(Methylamino)-1-(5-methylthiazol-2-yl)ethanol

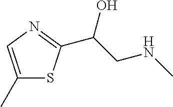

2-Chloro-1-(5-methylthiazol-2-yl)ethanol (Example 39b, 1.24 g, 7.0 mmol) was dissolved in a 33% solution of methanamine (17.3 ml, 140 mmol) in EtOH. The solution was heated to 80° C. for 3 h. The solvent and excess methylamine was evaporated at reduced pressure to give 1.659 g of crude product, which was used without further purification.

### Example 39d

### 2-(((2,6-Dichloropyridin-3-yl)methyl)(methyl)amino)-1-(5-methylthiazol-2

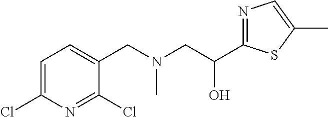

2-(Methylamino)-1-(5-methylthiazol-2-yl)ethanol HCl (Example 39c, 1.30 g, 6.23 mmol) was added to a solution of 3-(bromomethyl)-2,6-dichloropyridine (CAS 58596-59-1, 1.20 g, 4.98 mmol) in MeCN (20 mL) followed by the addition of TEA (1.39 mL, 9.96 mmol). The resulting mixture was stirred for 5 h at room temperature. The solvent was evaporated and the residue was partitioned between DCM and water, the aqueous phase was extracted with DCM and the combined extracts were dried over MgSO₄ and concentrated. The residue was purified by column chromatography on silica using gradient elution with increasing concentration of EtOAc, from 0 to 50% in heptane to give the title compound (1.13 g, 68%). ¹H NMR (500 MHz, CDCl₃) δ ppm 2.38 (s, 3H), 2.43-2.48 (m, 3H), 2.88 (m, 1H), 3.05 (m, 1H), 3.73 (d, 1H), 3.84 (d, 1H), 5.06 (dd, 1H), 7.30 (d, 1 H), 7.37 (m, 1H), 7.79 (d, 1H). MS m/z 332, 334, 336 [M+H]⁺.

### Example 39e

### 8-Chloro-4-methyl-2-(5-methylthiazol-2-yl)-2,3,4,5-tetrahydropyrido[3,2-f][1,4]oxazepine

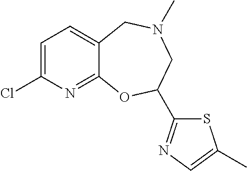

A solution of potassium 2-methylbutan-2-olate (1.7 M, 1.98 mL, 3.37 mmol) in toluene was added to a mixture of 2-(((2,6-dichloropyridin-3-yl)methyl)(methyl)amino)-1-(5-methylthiazol-2-yl)ethanol (Example 39d, 1.120 g, 3.4 mmol) in toluene (10 mL). The resulting mixture was stirred at r.t. for 16 h. The solvent was evaporated and the residue was purified by column chromatography on silica using gradient elution with increasing concentration of ethyl acetate, from 0 to 50% in heptane to give the title compound (0.504 g, 50%). ¹H NMR (500 MHz, CDCl₃) δ ppm 2.47-2.50 (m, 3H), 2.52 (s, 3H), 3.21 (dd, 1H), 3.62 (d, 1H), 3.73 (d, 1H), 3.95 (d, 1H), 5.34 (d, 1H), 7.13 (d, 1H), 7.41 (m, 1H), 7.55 (d, 1H). MS m/z 296, 298 [M+H]⁺.

### Example 40

### N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine

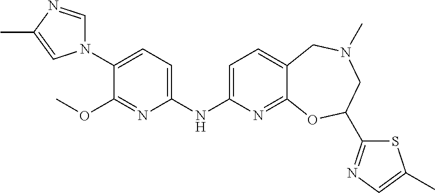

Preparation as in Example 39 using 8-chloro-4-methyl-2-(5-methylthiazol-2-yl)-2,3,4,5-tetrahydropyrido[3,2-f][1,4]oxazepine (Example 39e, 140 mg, 0.47 mmol) and 6-methoxy-5-(4-methyl-1H-imidazol-1-yl)pyridin-2-ylamine (Example 6a, 106 mg, 0.52 mmol) as starting materials gave the title compound (162 mg, 74%). ¹H NMR (500 MHz, CDCl₃) δ ppm 2.29-2.33 (m, 3H), 2.49-2.51 (m, 3H), 2.52 (s, 3H), 3.17 (dd, 1H), 3.60 (m, 1H), 3.64-3.70 (m, 1H), 3.93 (d, 1H), 4.00 (s, 3H), 5.31-5.36 (m, 1H), 6.85-6.89 (m, 2H), 7.31 (s, 1H), 7.43 (d, 1H), 7.46 (d, 1H), 7.54 (d, 1H), 7.57 (d, 1H), 7.66 (d, 1H). MS m/z 464 [M+H]⁺.

### Example 41

### N-(6-Methoxy-5-(1-methyl-1H-pyrazol-4-yl)pyridin-2-yl)-4-methyl-2-(5-methylpyridin-2-yl)-2,3,4,5-tetrahydropyrido[3,2-f][1,4]oxazepin-8-amine

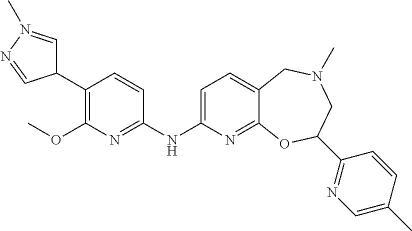

8-Chloro-4-methyl-2-(5-methylpyridin-2-yl)-2,3,4,5-tetrahydropyrido[3,2-f][1,4]oxazepine (Example 41d, 0.112 g, 0.55 mmol), 6-methoxy-5-(1-methyl-1H-pyrazol-4-yl)pyridin-2-ylamine (Example 2a, 0.112 g, 0.55 mmol), sodium tert-butoxide (0.079 g, 0.82 mmol), rac-2,2′-bis(diphenylphosphino)-1,1′-binaphthyl (0.034 g, 0.05 mmol) and tris(dibenzylideneacetone)dipalladium(0) (0.025 g, 0.03 mmol) were added to a microwave vial and toluene (3 mL) was added. The reaction mixture was flushed with nitrogen and the mixture was heated to 100° C. and stirred overnight. The solids were filtered off and washed with DCM. The organic solution was extracted by sat. NaHCO₃ solution. The organic layer was dried over anhydrous sodium sulfate, filtered and concentrated. The crude product was purified by column chromatography using DCM:[DCM:MeOH:NH₃=90:10:1]=100:0 to 20:80 as gradient to give the title compound (170 mg, 68%). ¹H NMR (500 MHz, CDCl₃) δ ppm 2.36 (s, 3H) 2.55 (br. s., 3H) 3.12-3.24 (m, 1H) 3.40-3.54 (m, 1H) 3.70-3.80 (m, 1H) 3.95 (s, 3H) 3.99-4.04 (m, 1H) 4.08 (s, 3H) 5.18-5.28 (m, 1H) 6.61 (d, 1H) 7.18 (s, 1H) 7.54 (d, 1H) 7.59 (d, 1H) 7.63-7.67 (m, 1 H) 7.68 (d, 1H) 7.77 (d, 1H) 7.80 (d, 2H) 8.40 (d, 1H). MS m/z 458 [M+H]⁺.

### Example 41a

### 2-Amino-1-(5-methylpyridin-2-yl)ethanol

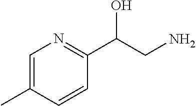

5-Methylpicolinaldehyde (CAS 4985-92-6, 3.5 g, 28.89 mmol) in dry DCM (70 mL) was set under N₂-atmosphere and cooled down to 0° C. To this mixture was added dropwise a DCM-solution (5 mL) of trimethylsilyl cyanide (4.62 mL, 34.67 mmol) and zinc iodide (9.22 mg, 0.03 mmol). After the addition was complete the mixture was allowed to warm up to r.t. under 1.5 h and then concentrated. The crude was diluted with ether (70 mL), cooled down to 0° C. and lithium aluminum hydride (1.426 g, 37.56 mmol) was added in two portions. The mixture was allowed to warm up to r.t. overnight then cooled back to 0° C. again and was treated with water (1.44 mL), 15% NaOH solution (1.44 mL), and water (4.32 mL). The resulting precipitates were filtered through Celite and washed with MeOH and ether. The filtrate was concentrated and the crude was purified by column chromatography using DCM:[MeOH (1% NH₃)]=100:0 to 40:60 gradient to give the title compound (1.57 mg, 36%). ¹H NMR (400 MHz, CDCl₃) δ ppm 2.32 (s, 3H) 2.93 (dd, 1H) 3.18 (dd, 1H) 4.78 (dd, 1H) 7.23-7.28 (m, 1H) 7.46-7.54 (m, 1H) 8.31-8.37 (m, 1H). MS m/z 153 [M+H]⁺.

### Example 41b

### 2-((2,6-Dichloropyridin-3-yl)methylamino)-1-(5-methylpyridin-2-yl)ethanol

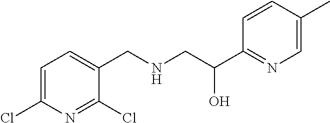

Potassium carbonate (3.56 g, 25.79 mmol) was added to a solution of 3-(bromomethyl)-2,6-dichloropyridine (CAS 58596-59-1, 2.485 g, 10.32 mmol) and 2-amino-1-(5-methylpyridin-2-yl)ethanol (Example 41a, 1.57 g, 10.32 mmol) in MeCN (30 mL)/DCM (15 mL) at room temperature and then stirred for 16 h. The solids were removed and the solvent was removed in vacuo. The crude product was purified by silica flash chromatography using DCM:[DCM:MeOH:NH₃=90:10:1]=100:0 to 40:60 as gradient to give the title compound (1.02 g, 32%). ¹H NMR (400 MHz, CDCl₃) δ ppm 2.31-2.39 (m, 3H) 2.92 (dd, 1H) 3.12 (dd, 1H) 3.98 (s, 2H) 4.92 (dd, 1H) 7.24-7.29 (m, 2H) 7.53 (ddd, 1H) 7.85 (d, 1H) 8.34-8.38 (m, 1H). MS m/z 312, 314 [M+H]⁺.

### Example 41c

### 8-Chloro-2-(5-methylpyridin-2-yl)-2,3,4,5-tetrahydropyrido[3,2-f][1,4]oxazepine

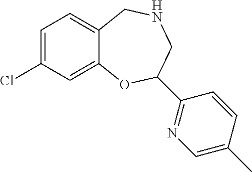

Sodium tert-butoxide (0.471 g, 4.90 mmol) was added in two portions to a stirred solution of 2-((2,6-dichloropyridin-3-yl)methylamino)-1-(5-methylpyridin-2-yl)ethanol (Example 41b, 1.02 g, 3.27 mmol) in THF (17 mL) at 0° C. The mixture was set under N₂-atmosphere, stirred for 5 min at 0° C. and was then allowed to warm-up to r.t. and stirred overnight. The temperature was raised to 45° C. and stirred for 4 hours. Sodium tert-butoxide (60 mg) was added and the mixture was stirred at 45° C. for 1 h and at r.t. overnight. Water was added to the reaction mixture and the phases were separated. The aqueous layer was extracted with ethyl acetate. The organic layer was dried over anhydrous sodium sulfate, filtered and concentrated. The residue was purified on a silica column using DCM:(DCM:MeOH:NH₃=90:10:1)=100:0 to 0:100 gradient to give the title compound (210 mg, 23%). ¹H NMR (400 MHz, CDCl₃) δ ppm 2.35 (s, 3H) 3.32 (dd, 1H) 3.74 (dd, 1H) 4.01-4.14 (m, 2H) 5.09 (dd, 1H) 7.07 (d, 1H) 7.51 (d, 1H) 7.55-7.59 (m, 1H) 7.61-7.67 (m, 1H) 8.33-8.38 (m, 1H). MS m/z 277, 279 [M+H]⁺.

### Example 41d

### 8-Chloro-4-methyl-2-(5-methylpyridin-2-yl)-2,3,4,5-tetrahydropyrido[3,2-f][1,4]oxazepine

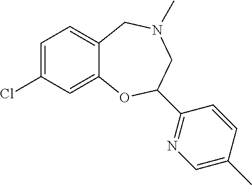

Formaldehyde (37%, 0.567 mL, 7.62 mmol) and acetic acid (0.022 mL, 0.38 mmol) were added to a MeOH— solution (3 mL) of 8-chloro-2-(5-methylpyridin-2-yl)-2,3,4,5-tetrahydropyrido[3,2-f][1,4]oxazepine (Example 41c, 0.21 g, 0.76 mmol) at r.t. under N₂-atmosphere. The mixture was stirred for 30 min and then sodium cyanoborohydride (0.072 g, 1.14 mmol) was added and the reaction stirred overnight at r.t. MeOH (containing 1% NH₃) was added to adjust the pH to 7 and then the solvent was removed in vacuo. Sat. NaHCO₃ solution and ethyl acetate were added to the crude oil and the phases were separated. The aqueous layer was dried over anhydrous sodium sulfate, filtered and concentrated. The crude product was purified by silica flash chromatography using DCM:[DCM:MeOH:NH₃=90:10:1]=100:0 to 20:80 as gradient to give the title compound (159 mg, 72%). ¹H NMR (400 MHz, CDCl₃) δ ppm 2.35 (s, 3H) 2.52 (s, 3H) 3.14 (br. s., 1H) 3.49 (d, 1H) 3.77 (d, 1H) 3.96 (d, 1H) 5.20 (d, 1H) 7.11 (d, 1H) 7.52-7.60 (m, 2H) 7.67 (d, 1H) 8.38 (dd, 1H). MS m/z 290, 292 [M+H]⁺.

### Example 42

### N-(6-Methoxy-5-(1-methyl-1H-pyrazol-4-yl)pyridin-2-yl)-4-methyl-2-(4-methylpyridin-2-yl)-2,3,4,5-tetrahydropyrido[3,2-f][1,4]oxazepin-8-amine

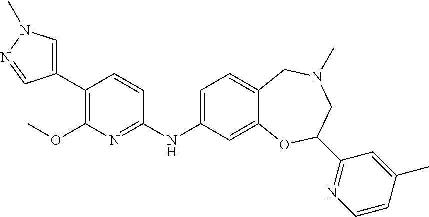

6-Methoxy-5-(1-methyl-1H-pyrazol-4-yl)pyridin-2-ylamine (Example 2a, 66.3 mg, 0.32 mmol), 8-chloro-4-methyl-2-(4-methylpyridin-2-yl)-2,3,4,5-tetrahydropyrido[3,2-f][1,4]oxazepine (Example 42d, 94 mg, 0.32 mmol), sodium tert-butoxide (46.8 mg, 0.49 mmol), rac-2,2′-bis(diphenylphosphino)-1,1′-binaphthyl (20.2 mg, 0.03 mmol) and tris(dibenzylideneacetone)dipalladium(0) (14.8 mg, 0.02 mmol) were added to a microwave vial and then toluene (2 mL) was added. The reaction mixture was flushed with nitrogen and the mixture was heated to 100° C. and stirred overnight. The solids were filtered off and washed with DCM. The organic solution was extracted by sat. NaHCO₃ solution. The organic layer was dried over anhydrous sodium sulfate, filtered and concentrated. The crude product was purified by column chromatography using DCM:[DCM:MeOH:NH₃=90:10:1]=100:0 to 20:80 as gradient to give the title compound (64 mg, 43%). ¹H NMR (400 MHz, CDCl₃) δ ppm 2.38 (s, 3H) 2.50 (s, 3H) 3.09 (dd, 1H) 3.41 (d, 1H) 3.69 (d, 1H) 3.93 (s, 3H) 3.96 (d, 1H) 4.06 (s, 3H) 5.15-5.23 (m, 1H) 6.53 (d, 1H) 7.02 (d, 1H) 7.37 (s, 1H) 7.52 (d, 1H) 7.57 (s, 1H) 7.64 (d, 1H) 7.75-7.83 (m, 3H) 8.40 (d, 1H). MS m/z 458 [M+H]⁺.

### Example 42a

### 2-Amino-1-(4-methylpyridin-2-yl)ethanol

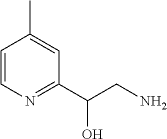

4-Methylpicolinaldehyde (CAS 53547-60-7, 3.5 g, 28.9 mmol) in dry DCM (70 mL) was set under N₂-atmosphere and cooled down to 0° C. To this mixture was added dropwise a DCM (5 mL) solution of trimethylsilyl cyanide (4.62 mL, 34.7 mmol) and zinc iodide (9.2 mg, 0.03 mmol). After the addition was complete the mixture was allowed to warm up to r.t. under 1.5 hours and then concentrated. The crude was diluted with ether (70 mL), cooled down to 0° C. and lithium aluminum hydride (1.43 g, 37.6 mmol) was added in two portions. The mixture was allowed to warm up to r.t. overnight and then cooled back to 0° C. and was treated with water (1.44 mL), 15% NaOH solution (1.44 mL), and water (4.3 mL). The resulting precipitates were filtered through Celite and washed with MeOH. The filtrate was concentrated and the crude was purified by column chromatography using DCM:[MeOH (1% NH₃)]=100:0 to 50:50 gradient to give the title compound (1.47 g, 33%). ¹H NMR (400 MHz, CDCl₃) δ (ppm) 2.32-2.38 (m, 3H) 2.96 (dd, 1H) 3.23 (dd, 1H) 4.80 (dd, 1 H) 7.01 (d, 1H) 7.20 (d, 1H) 8.36 (d, 1H). MS m/z 153 [M+H]⁺.

### Example 42b

### 2-((2,6-Dichloropyridin-3-yl)methylamino)-1-(4-methylpyridin-2-yl)ethanol

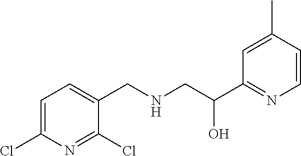

Potassium carbonate (3.34 g, 24.15 mmol) was added to a DCM (15 mL)/MeCN (30 mL) solution of 2-amino-1-(4-methylpyridin-2-yl)ethanol (Example 42a, 1.47 g, 9.66 mmol) and 3-(bromomethyl)-2,6-dichloropyridine (CAS 58596-59-1, 2.33 g, 9.66 mmol) at room temperature and then stirred for 16 h. The solids were removed and the solvent was removed in vacuo. The crude product was purified by silica flash chromatography using DCM:[DCM:MeOH:NH₃=90:10:1]=100:0 to 40:60 as gradient to give the title compound (0.74 g, 25%). ¹H NMR (400 MHz, CDCl₃) δ ppm 2.38 (s, 3

H) 2.84 (dd, 1H) 3.06 (dd, 1H) 3.91 (s, 2H) 4.85 (dd, 1H) 7.06 (d, 1H) 7.16 (d, 1H) 7.24-7.28 (m, 1H) 7.76 (d, 1H) 8.40 (d, 1H). MS m/z 312, 314 [M+H]⁺.

### Example 42c

### 8-Chloro-2-(4-methylpyridin-2-yl)-2,3,4,5-tetrahydropyrido[3,2-f][1,4]oxazepine

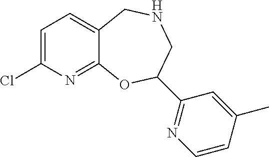

Sodium tert-butoxide (0.342 g, 3.56 mmol) was added in two portions to a stirred solution of 2-((2,6-dichloropyridin-3-yl)methylamino)-1-(4-methylpyridin-2-yl)ethanol (Example 42b, 0.74 g, 2.37 mmol) in THF (13 mL) at 0° C. The mixture was set under N₂-atmosphere, stirred for 5 min at 0° C. and was then allowed to warm-up to r.t. and stirred for 2 days. Thereafter water was added to the reaction mixture and the phases were separated. The aqueous layer was extracted with ethyl acetate. The organic layer was dried over anhydrous sodium sulfate, filtered and concentrated. The residue was purified on silica column using DCM:(DCM:MeOH:NH₃=90:10:1)=100:0 to 0:100 gradient to give the title compound (168 mg, 26%). ¹H NMR (400 MHz, CDCl₃) δ ppm 2.40 (s, 3 H) 3.24 (dd, 1H) 3.74 (dd, 1H) 3.98-4.08 (m, 2H) 5.04 (dd, 1H) 7.05 (d, 1H) 7.08 (d, 1H) 7.51 (d, 1H) 7.60 (d, 1H) 8.39 (d, 1H). MS m/z 277 [M+H]⁺.

### Example 42d

### 8-Chloro-4-methyl-2-(4-methylpyridin-2-yl)-2,3,4,5-tetrahydropyrido[3,2-f][1,4]oxazepine

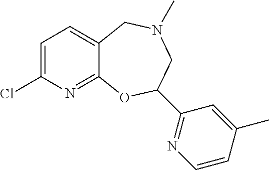

Formaldehyde (37%, 0.454 mL, 6.09 mmol) and acetic acid (0.017 mL, 0.30 mmol) were added to the MeOH (2 mL) solution of 8-chloro-2-(4-methylpyridin-2-yl)-2,3,4,5-tetrahydropyrido[3,2-f][1,4]oxazepine (Example 42c, 0.168 g, 0.61 mmol) at rt under N₂-atmosphere. The mixture was stirred for 30 min and then sodium cyanoborohydride (0.057 g, 0.91 mmol) was added and allowed to stir for 1 h at r.t. MeOH (containing 1% NH₃) was added to adjust the pH to 7 and the solvent was removed in vacuo. Sat. NaHCO₃ solution and ethyl acetate were added to the crude oil and the phases were separated. The aqueous layer was dried over anhydrous sodium sulfate, filtered and concentrated. The crude product was purified by silica flash chromatography using DCM:[DCM:MeOH:NH₃=90:10:1]=100:0 to 20:80 as gradient to give the title compound (99 mg, 56%). ¹H NMR (400 MHz, CDCl₃) δ ppm 2.41 (s, 3H) 2.54 (s, 3H) 3.10-3.21 (m, 1H) 3.47-3.58 (m, 1H) 3.81 (s, 1H) 3.98 (d, 1H) 5.21 (d, 1H) 7.05 (d, 1H) 7.12 (d, 1H) 7.56 (d, 1H) 7.62 (d, 1H) 8.41 (d, 1H). MS m/z 290, 292 [M+H]⁺.

### Example 43

### 5-(2-Methoxy-6-(4-methyl-2-(2,2,2-trifluoroethyl)-2,3,4,5-tetrahydropyrido[3,2-f][1,4]oxazepin-8-ylamino)pyridin-3-yl)-1-methylpyridin-2(1H)-one; isomer 1

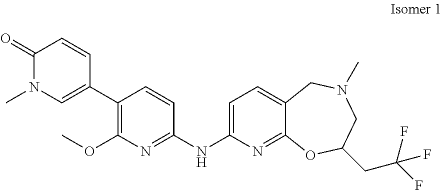

A mixture of 5-(6-amino-2-methoxypyridin-3-yl)-1-methylpyridin-2(1H)-one (Example 43a, 0.2 g, 0.86 mmol), 8-chloro-4-methyl-2-(2,2,2-trifluoroethyl)-2,3,4,5-tetrahydropyrido[3,2-f][1,4]oxazepine (Example 3b, 0.243 g, 0.86 mmol), palladium(II) acetate (0.019 g, 0.09 mmol), 9,9-dimethyl-4,5-bis(diphenylphosphino)xanthene (0.050 g, 0.09 mmol) and sodium tert-pentoxide (0.114 g, 1.04 mmol) in dioxane (1.5 mL) was heated by microwave irradiation to 120° C. for 30 min. The mixture was allowed to cool. DCM (5 mL) was added and the mixture was filtered through a short pad of Celite. The filtrate was collected and the solvent was removed by rotary evaporation. The crude product was added to a silica gel column and was eluted with 0-5% MeOH in DCM. The collected fractions were combined and the solvent was removed by rotary evaporation. The residue was purified by chiral SFC (Chiralcel OD-H column; 4.6*250 mm; 5 μm using methanol/CO₂ (20:80) as eluent at a flow rate of 50 mL/min) to yield 5-(2-methoxy-6-(4-methyl-2-(2,2,2-trifluoroethyl)-2,3,4,5-tetrahydropyrido[3,2-f][1,4]oxazepin-8-ylamino)pyridin-3-yl)-1-methylpyridin-2(1H)-one, isomer 1, the first isomer to elute (0.055 g, 27%). ¹H NMR (500 MHz, DMSO-d₆) δ ppm 2.31 (br. s., 3H) 2.67 (m, 2H) 2.83 (br. s., 1H) 2.95 (d, 1H) 3.47 (s, 3H) 3.61 (br. s., 2H) 3.92 (s, 3H) 4.30 (t, 1H) 6.41 (d, 1H) 7.25 (d, 1H) 7.58 (m, 3H) 7.66 (dd, 1H) 7.87 (d, 1H) 9.66 (s, 1H). MS (ES+) m/z 476.1 [M+H]⁺.

### Example 43a

### 5-(6-Amino-2-methoxypyridin-3-yl)-1-methylpyridin-2(1H)-one

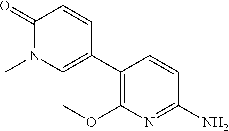

1-Methyl-5-(4,4,5,5-tetramethyl-1,3,2-dioxaborolan-2-yl)pyridin-2(1H)-one (CAS 1002309-52-5, 1.088 g, 4.63 mmol), 5-bromo-6-methoxypyridin-2-amine (CAS 1211533-83-30, 94 g, 4.63 mmol), 1,1′-bis(diphenylphosphino)ferrocene-palladium(II)chloride dichloromethane complex (0.113 g, 0.14 mmol) and potassium carbonate (aqueous 2M) (6 mL, 12.00 mmol) in dioxane (10 mL) were heated under argon to 80° C. for 1 h. The mixture was allowed to cool and was filtered through a short pad of Celite. The pad was washed with EtOAc (100 mL). The filtrate was collected and the solvent was removed by rotary evaporation. The crude product was added to a silica gel column and was eluted with 0-3% MeOH in DCM. The collected fractions were combined and the solvent was removed by rotary evaporation. The residue was redissolved in DCM and the mixture was washed with saturated aqueous Na₂CO₃, dried over K₂CO₃, filtered and the solvent was removed by rotary evaporation to yield the title compound (0.402 g, 37%). ¹H NMR (500 MHz, DMSO-d₆) δ ppm 3.44 (s, 3H) 3.78 (s, 3H) 5.98 (s, 2H) 6.07 (d, 1H) 6.37 (d, 1H) 7.33 (d, 1H) 7.56 (dd, 1H) 7.72 (d, 1H). MS (ES+) m/z 232.1 [M+H]⁺.

### Example 44

### 5-(2-Methoxy-6-(4-methyl-2-(2,2,2-trifluoroethyl)-2,3,4,5-tetrahydropyrido[3,2-f][1,4]oxazepin-8-ylamino)pyridin-3-yl)-1-methylpyridin-2(1H)-one; isomer 2

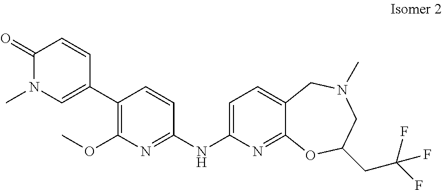

Separation as in Example 43 gave 5-(2-methoxy-6-(4-methyl-2-(2,2,2-trifluoroethyl)-2,3,4,5-tetrahydropyrido[3,2-f][1,4]oxazepin-8-ylamino)pyridin-3-yl)-1-methylpyridin-2(1H)-one, isomer 2, the second isomer to elute (0.051 g, 25%). ¹H NMR (500 MHz, DMSO-d₆) δ ppm 2.32 (m, 3H) 2.67 (m, 2H) 2.83 (m, 1H) 2.96 (m, 1H) 3.47 (s, 3H) 3.61 (d, 2H) 3.92 (s, 3H) 4.30 (br. s., 1H) 6.41 (d, 1H) 7.25 (d, 1H) 7.58 (m, 3H) 7.66 (dd, 1H) 7.87 (d, 1H) 9.66 (br. s., 1H). MS (ES+) m/z 476.1 [M+H]⁺.

### Example 45

### (R)-5-(2-Methoxy-6-(4-methyl-2-phenyl-2,3,4,5-tetrahydropyrido[3,2-f][1,4]oxazepin-8-ylamino)pyridin-3-yl)-1-methylpyridin-2(1H)-one

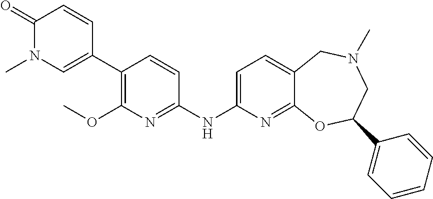

(R)-8-Chloro-4-methyl-2-phenyl-2,3,4,5-tetrahydropyrido[3,2-f][1,4]oxazepine (Example 14c, 300 mg, 1.09 mmol), 5-(6-amino-2-methoxypyridin-3-yl)-1-methylpyridin-2(1H)-one (Example 43a, 253 mg, 1.09 mmol), palladium(II) acetate (24.5 mg, 0.11 mmol), 9,9-dimethyl-4,5-bis(diphenylphosphino)xanthene (63.2 mg, 0.11 mmol) and sodium tert-pentoxide (144 mg, 1.31 mmol) were mixed in dioxane (3 mL) and run in a microwave reactor for 40 min at 120° C. The mixture was filtered through a pad of Celite and concentrated. The residue was purified using flash column chromatography using 0-6% MeOH (1% NH₃) in DCM to yield title compound (180 mg, 35%). ¹H NMR (400 MHz, CDCl₃) δ ppm 2.53 (br. s., 3H) 3.13 (br. s., 2H) 3.61 (s, 3H) 3.65-3.80 (m, 1H) 3.90 (br. s., 1H) 3.97-4.07 (m, 3H) 5.14 (br. s., 1H) 6.57-6.67 (m, 2H) 7.13-7.26 (m, 1H) 7.30-7.37 (m, 1H) 7.37-7.45 (m, 3H) 7.45-7.50 (m, 2H) 7.50-7.60 (m, 3H) 7.74 (d, 1H), MS (ES+) m/z 470 [M+H]⁺.

### Example 46

### N-[6-Methoxy-5-(2-methylpyrimidin-4-yl)-2-pyridyl]-4-methyl-2-(2,2,2-trifluoroethyl)-3,5-dihydro-2H-pyrido[3,2-f][1,4]oxazepin-8-amine

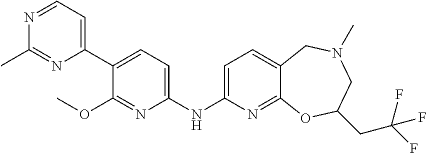

Pd(OAc)₂ (34 mg, 0.15 mmol) and Xantphos (174 mg, 0.30 mmol) were added to a degassed mixture of 8-chloro-4-methyl-2-(2,2,2-trifluoroethyl)-2,3,4,5-tetrahydropyrido[3,2-f][1,4]oxazepine (Example 3b, 142 mg, 0.50 mmol), 6-methoxy-5-(2-methyl-pyrimidin-4-yl)-pyridin-2-ylamine (46a, 110 mg, 0.50 mmol) and cesium carbonate (212 mg, 0.65 mmol) in dioxane (12 mL) and the reaction mixture was heated in a microwave reactor for 1.5 h at 145° C. The reaction mixture was cooled to room temperature, diluted with ethyl acetate (50 mL), filtered and concentrated in vacuo. The residue was purified by flash column chromatography using 5% MeOH in DCM to afford 52 mg (23%) of the title compound. ¹H NMR (400 MHz, CDCl₃) δ ppm 2.33-2.43 (m, 1H) 2.45 (s, 3 H) 2.70 (d, 1H) 2.75 (s, 3H) 2.91-3.03 (m, 2H) 3.61 (d, 1H) 3.76 (d, 1H) 4.09 (s, 3H) 4.43 (d 1H) 6.81 (d, 1H) 7.32 (s, 1H) 7.50 (d, 1H) 7.71 (s, 1H) 7.87 (d, 1H) 8.50-8.60 (m, 2H). ¹⁹F NMR (376 MHz, CDCl₃) δ ppm −63.76. ESMS m/z 461.1 [M+H]⁺.

### Example 46a

### 6-Methoxy-5-(2-methyl-pyrimidin-4-yl)-pyridin-2-ylamine

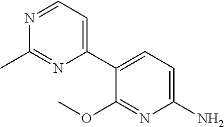

PdCl₂(PPh₃)₂ (413 mg, 0.58 mmol) was added to a degassed mixture of 4-bromo-2-methyl-pyrimidine (1.02 g, 5.89 mmol), 6-methoxy-5-(4,4,5,5-tetramethyl-[1,3,2]dioxaborolan-2-yl)-pyridin-2-ylamine 2 (Example 7c, 2.2 g, 8.79 mmol) and K₂CO₃ (2.43 g, 17.6 mmol) in a mixture of DME, EtOH and H₂O (6:2:1, 200 mL). The reaction mixture was heated in a sealed tube for 1 hour at 100° C. The mixture was cooled to room temperature, diluted with ethyl acetate and filtered. The volatiles were removed in vacuo and the residue was purified by flash column chromatography using a gradient of 5 to 10% EtOAc in DCM to afford 610 mg (48%) of the title compound. ¹H NMR (400 MHz, CD₃OD) δ ppm 2.74 (s, 3H) 4.06 (s, 3H) 6.29 (d, 1H) 8.27 (d, 1H) 8.45 (d, 1H) 8.55 (d, 1H). ESMS m/z 217.0 [M+H]⁺.

### Example 47

### N-[6-Methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine

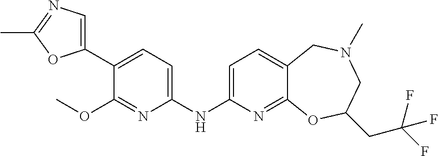

6-Methoxy-5-(2-methyl-oxazol-5-yl)-pyridin-2-ylamine (Example 47c, 323 mg, 1.57 mmol) was added to a solution of 8-chloro-4-methyl-2-(2,2,2-trifluoroethyl)-2,3,4,5-tetrahydropyrido[3,2-f][1,4]oxazepine (Example 3b, 442 mg, 1.57 mmol) in anhydrous 1,4-dioxane (20 mL) and the mixture was degassed for 20 minutes using nitrogen. Palladium acetate (53 mg, 0.24 mmol), Xantphos (274 mg, 0.47 mmol) and cesium carbonate (667 mg, 2.05 mmol) were then added and the reaction mixture was purged with nitrogen for an additional 10 minutes. The reaction mixture was heated in a microwave reactor at 145° C. for 1 hour, cooled to room temperature and concentrated under reduced pressure. The residue was purified by flash chromatography using a gradient of 0 to 2% methanol in dichloromethane to afford 290 mg of the title compound (41%). ¹H NMR (400 MHz, CD₃OD) δ ppm 2.43 (s, 3H) 2.47-2.61 (m, 4H) 2.62-2.78 (m, 1H) 2.85-2.94 (m, 1H) 2.99-3.07 (m, 1H) 3.62-3.75 (m, 2H) 4.08 (s, 3H) 4.32-4.41 (m, 1H) 7.21 (s, 1H) 7.26 (d, 1 H) 7.49-7.55 (m, 1H) 7.55-7.60 (m, 1H) 7.88 (d, 1H). ESMS m/z 448 [M−1]⁻.

### Example 47a

### 2-Methyl-oxazole

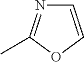

2-Methyl-oxazole-4-carboxylic acid (CAS 23012-10-4, 10.00 g, 78.68 mmol) was dissolved in freshly distilled quinoline (30 mL). Copper (II) oxide (30 mg, catalytic) was added and the reaction vessel was equipped with a distillation apparatus. The reaction mixture was heated at −200° C. until a clear liquid started to distill (head of column at 80-120° C., atmospheric pressure). The crude product (5.63 g) was purified by distillation (oil bath at 115-120° C., head of column at 80° C., atmospheric pressure) to afford 3.50 g (54%) of 2-methyl-oxazole. ¹H NMR (400 MHz, CDCl₃) δ ppm 2.47 (s, 3H) 7.00 (s, 1H) 7.54 (s, 1H).

### Example 47b

### Trimethyl-(2-methyloxazol-5-yl)stannane

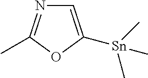

n-BuLi (2.5M in hexanes, 11.6 mL, 29.1 mmol) was added dropwise over 5 minutes to a solution of 2-methyl-oxazole (Example 47a, 1.86 g, 22.4 mmol) in anhydrous diethyl ether (40 mL) at −78° C. The reaction mixture was stirred for 1 hour at −78° C., then allowed to warm to 0° C. and stirred for 1 hour. The reaction mixture was then cooled to −78° C. and a solution of chlorotrimethylstannane (4.01 g, 20.15 mmol) in anhydrous diethyl ether (20 mL) was added dropwise over 5 minutes. The reaction mixture was stirred for 30 minutes and then allowed to warm to room temperature and stirred overnight. Water (20 mL) was added and the mixture was extracted with ethyl acetate (3×20 mL). The combined organic extracts were dried over sodium sulfate, filtered and concentrated under reduced pressure to provide trimethyl-(2-methyloxazol-5-yl)stannane which was used in the next step without further purification.

### Example 47c

### 6-Methoxy-5-(2-methyl-oxazol-5-yl)-pyridin-2-ylamine

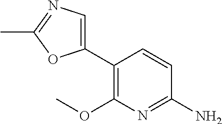

Tetrakis(triphenylphosphine)palladium(0) (0.43 g, 0.37 mmol) was added to a degassed solution of trimethyl-(2-methyloxazol-5-yl)stannane (Example 47b, 22.4 mmol) and 5-bromo-6-methoxy-pyridin-2-ylamine (Example 7b, 1.5 g, 7.4 mmol) in o-xylene (120 mL). The reaction mixture was heated in a sealed tube at 140° C. overnight. The reaction mixture was then cooled to room temperature and concentrated under reduced pressure. The residue was purified by flash chromatography eluting with a gradient of 10 to 50% ethyl acetate in hexanes to afford 6-methoxy-5-(2-methyl-oxazol-5-yl)-pyridin-2-ylamine (0.55 g, 36%). ¹H NMR (400 MHz, CDCl₃) δ ppm 2.48 (s, 3H) 3.99 (s, 3H) 4.41 (br. s., 2H) 6.15 (d, 1H) 7.18 (s, 1H) 7.74 (d, 1H). ESMS m/z 206 [M+H]⁺.

### Example 48

### 6-[(2-Ethyl-4-methyl-3,5-dihydro-2H-pyrido[3,2-f][1,4]oxazepin-8-yl)amino]-3-(4-methylimidazol-1-yl)pyridine-2-carbonitrile

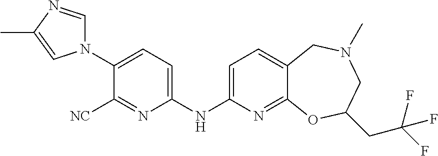

6-Amino-3-(4-methyl-imidazol-1-yl)-pyridine-2-carbonitrile (Example 48c, 213 mg, 1.07 mmol) was added to a solution of 8-chloro-4-methyl-2-(2,2,2-trifluoroethyl)-2,3,4,5-tetrahydropyrido[3,2-f][1,4]oxazepine (Example 3b 300 mg, 1.07 mmol) in anhydrous 1,4-dioxane (15 mL) and the mixture was degassed for 20 minutes using nitrogen. Palladium acetate (36 mg, 0.16 mmol), Xantphos (186 mg, 0.32 mmol) and cesium carbonate (453 mg, 1.39 mmol) were added and the reaction mixture was degassed for an additional 10 minutes. The reaction mixture was heated in a microwave reactor at 145° C. for 1 hour, cooled to room temperature and concentrated under reduced pressure. The residue was purified by first flash chromatography eluting with a gradient of 0 to 5% methanol in dichloromethane and then by preparative HPLC to give the title compound (99.7 mg, 21%). ¹H NMR (400 MHz, CD₃OD) δ ppm 2.27 (s, 3H) 2.44 (s, 3H) 2.46-2.60 (m, 1H) 2.61-2.77 (m, 1H) 2.85-2.96 (m, 1H) 2.98-3.06 (m, 1H) 3.63-3.76 (m, 2H) 4.31-4.41 (m, 1H) 7.19 (s, 1H) 7.35 (d, 1H) 7.63 (d, 1H) 7.81 (d, 1H) 7.92 (s, 1H) 8.20 (d, 1H). ESMS m/z 442 [M−1]⁻.

### Example 48a

### 6-Amino-pyridine-2-carbonitrile

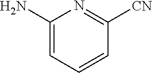

To a mixture of 2-amino-6-bromo-pyridine (22.9 g, 132 mmol), zinc dust (2.06 g, 31.7 mmol) and zinc cyanide (10.1 g, 86.0 mmol) in anhydrous N,N-dimethylacetamide (230 mL), dppf (2.97 g, 5.29 mmol) and tris(dibenzylideneacetone)dipalladium (2.40 g, 2.65 mmol) were added. The reaction mixture was degassed using nitrogen for 20 minutes and then heated in a sealed tube at 95° C. for 3 hours. Water (100 mL) was added and the mixture was extracted with ethyl acetate (3×100 mL). The combined organic extracts were washed with brine (100 mL), dried over sodium sulfate, filtered and concentrated under reduced pressure. The residue was purified by flash chromatography using a gradient of 10 to 50% ethyl acetate in hexanes to afford 10.50 g (67%) of the title compound. ¹H NMR (400 MHz, DMSO-d₆) δ ppm 6.56 (br. s., 2H) 6.69 (d, 1H) 7.02 (d, 1H) 7.46-7.55 (m, 1 H).

### Example 48b

### 6-Amino-3-bromo-pyridine-2-carbonitrile

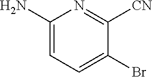

6-Amino-pyridine-2-carbonitrile (Example 48a, 10.5 g, 88.1 mmol) was dissolved in a mixture of dichloromethane and methanol (1:1, 200 mL). Tetrabutylammonium tribromide (51.0 g, 105.8 mmol) was added in one portion and the reaction mixture was stirred at room temperature for 2 hours. The volatiles were removed under reduced pressure and the resulting residue was triturated with dichloromethane (200 mL) and the precipitated product was collected by filtration. The filtrate was concentrated in vacuo and the residue was triturated again with dichloromethane, and the product was collected by filtration to afford 10 g (combined batches) of the product. The filtrate was concentrated in vacuo and the residue was purified by flash chromatography using a gradient of 10 to 50% ethyl acetate in hexanes to provide additional 3.04 g of 6-amino-3-bromo-pyridine-2-carbonitrile, giving a total yield of 13.0 g, (67%). ¹H NMR (400 MHz, DMSO-d₆) δ ppm 6.66 (d, 1 H) 6.80 (br. s., 2H) 7.73 (d, 1H). ESMS m/z 198, 200 [M+H]⁺.

### Example 48c

### 6-Amino-3-(4-methylimidazol-1-yl)pyridine-2-carbonitrile

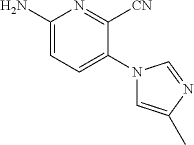

4-Methylimidazole (7.46 g, 90.9 mmol), copper iodide (2.31 g, 12.1 mmol) and cesium carbonate (29.6 g, 90.9 mmol) were added to a solution of 6-amino-3-bromo-pyridine-2-carbonitrile (Example 48b, 6.0 g, 30 mmol) in DMF (65 mL). The resulting suspension was degassed using vacuum and purged with nitrogen. The reaction mixture was stirred at room temperature for 30 minutes and then heated in a sealed tube at 120° C. overnight. The reaction mixture was cooled to room temperature, brine (100 mL) and ethyl acetate (100 mL) were added, and the obtained mixture was filtered through a pad of Celite. The filtrate was transferred to a reparatory funnel and the phases were separated. The organic layer was washed with brine (3×100 mL), dried over sodium sulfate, filtered and concentrated under reduced pressure. The residue was taken in a mixture of dichloromethane (50 mL) and methanol (10 mL), and the precipitate was collected by filtration. The filtrate concentrated in vacuo and the residue was purified by flash chromatography using a gradient of 2 to 10% methanol in dichloromethane giving a combined yield of 6-amino-3-bromo-pyridine-2-carbonitrile of 1.86 g (31%). ¹H NMR (400 MHz, DMSO-d₆) δ ppm 2.14 (s, 3H) 6.80 (d, 1H) 6.89 (s, 2H) 7.15 (br. s., 1H) 7.60 (d, 1H) 7.80 (br. s., 1H). ESMS m/z 200 [M+H]⁺.

### Example 49

### (2R)—N-[6-Methoxy-5-(2-methyloxazol-5-yl)-2-pyridyl]-4-methyl-2-phenyl-3,5-dihydro-2H-pyrido[3,2-f][1,4]oxazepin-8-amine

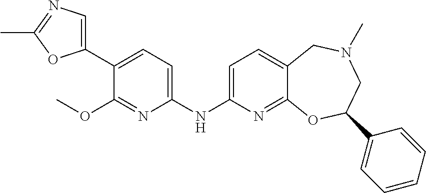

Palladium acetate (19 mg, 0.08 mmol), Xantphos (97 mg, 0.17 mmol) and cesium carbonate (236 mg, 0.72 mmol) were added to a degassed mixture of (2R)-8-chloro-4-methyl-2-phenyl-3,5-dihydro-2H-pyrido[3,2-f][1,4]oxazepine (Example 14c, 153 mg, 0.56 mmol) and 6-methoxy-5-(2-methyl-oxazol-5-yl)-pyridin-2-ylamine (Example 47c, 114 mg, 0.56 mmol) in anhydrous 1,4-dioxane (9 mL). The reaction mixture was purged with nitrogen for an additional 10 minutes and then heated in a microwave reactor at 145° C. for 1 hour. The reaction mixture was cooled to room temperature and concentrated under reduced pressure. The residue was purified by flash chromatography eluting with a gradient of 0 to 2% methanol in dichloromethane to afford a product which was further purified by crystallization from diethyl ether. The collected solid was washed with pentane and dried in vacuo to provide 78.6 mg (41%) of the title compound. ¹H NMR (400 MHz, CD₃OD) δ ppm 2.48 (s, 6H) 3.09 (d, 2H) 3.73-3.87 (m, 2H) 4.09 (s, 3H) 5.07-5.13 (m, 1H) 7.02 (d, 1H) 7.20 (s, 1H) 7.31-7.37 (m, 1H) 7.38-7.44 (m, 2H) 7.50 (d, 2H) 7.62-7.67 (m, 1H) 7.67-7.73 (m, 1H) 7.87 (d, 1 H). ESMS m/z 444 [M−1]⁻.

### Example 50

### (2S)-2-(5-Fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine; isomer 1

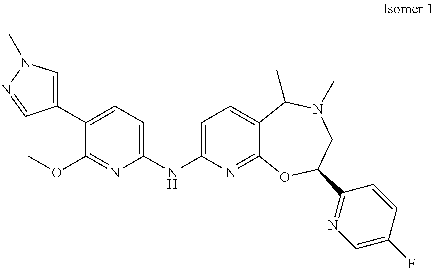

Chiral separation of (2S)-2-(5-fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine (Example 50f, 138 mg, 0.290 mmol) using SFC chromatography [Column: Chiralcel OD-H (4.6*250 mm; 5 μm) Mobile phase: 20% MeOH: 80% CO₂; Flow 50 mL/min] yielded 47 mg (34%) of isomer 1, the first isomer to elute.

### Example 50a

### 2-Chloro-1-(5-fluoro-2-pyridyl)ethanone

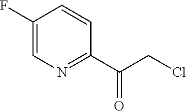

2-Bromo-5-fluoropyridine (3.88 g, 22 mmol) dissolved in toluene (10 mL) was added slowly to a solution of isopropylmagnesium chloride (13.2 mL, 26.4 mmol, 2M in THF) in toluene (30 mL) at room temperature. The reaction mixture was stirred for 3.5 hours at room temperature, cooled to 0° C. and a solution of 2-chloro-N-methoxy-N-methyl-acetamide (3.64 g, 26.4 mmol) in toluene (10 mL) was added slowly. The reaction mixture was stirred for 2.5 hours at 0° C., quenched with saturated ammonium chloride, diluted with ethyl acetate and saturated sodium bicarbonate and extracted with ethyl acetate (2×100 mL). The combined extracts were dried over anhydrous magnesium sulfate, filtered and concentrated in vacuo to afford 3.75 g (98%) of the title compound which was used in the next step without further purification. ¹H NMR (400 MHz, CDCl₃) δ ppm 5.07 (s, 2H), 7.56 (td, 1H), 8.17 (dd, 1H), 8.50 (d, 1H).

### Example 50b

### (1R)-2-Chloro-1-(5-fluoro-2-pyridyl)ethanol

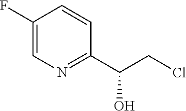

A solution of Noyoris catalyst (Angew. Chem. Int. Eng. 1997, (36) 285-288; 90 mg, 0.15 mmol) in DMF (10 mL) was cooled to 0° C. 2-Chloro-1-(5-fluoro-pyridin-2-yl)-ethanone (Example 50a, 500 mg, 2.89 mmol) was added followed by a mixture of formic acid and TEA (5:2, 1 mL). The reaction mixture was stirred for 5 minutes at 0° C. and then at room temperature for 45 minutes. Methanol (5 mL) was added and the resulting mixture was stirred for 5 minutes. The mixture was then concentrated in vacuo to approximately 50% of the volume, diluted with saturated sodium bicarbonate and ethyl acetate, and extracted with ethyl acetate (2×100 mL). The combined extracts were washed with water and brine, dried over anhydrous magnesium sulfate, filtered and concentrated in vacuo. The residue was purified by flash column chromatography using a gradient of 20 to 30% ethyl acetate in hexane to afford 358 mg (71%) of the title compound. ¹H NMR (400 MHz, CDCl₃) δ ppm 3.84 (m, 3H), 4.97 (m, 1H), 7.46 (m, 2H), 8.44 (s, 1H).

### Example 50c

### (1S)-1-(5-Fluoro-2-pyridyl)-2-(methylamino)ethanol

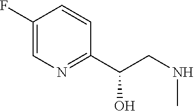

Methylamine (2 mL, 33% by weight in ethanol) was added to a solution of (1R)-2-chloro-1-(5-fluoro-pyridin-2-yl)-ethanol (Example 50b, 358 mg, 2.05 mmol) in ethanol (20 mL) and the reaction mixture was heated in a sealed tube at 60° C. for 48 hours. The mixture was cooled to room temperature, diluted with ethyl acetate, filtered and concentrated in vacuo to afford 390 mg which was used in the next step without further purification. ¹H NMR (400 MHz, CDCl₃) δ ppm 2.77 (s, 3H), 3.16 (dd, 1H), 3.40 (dd, 1H), 3.72 (m, 1H), 5.30 (m, 1H), 7.45 (m, 1H), 7.61 (m, 1H), 8.33 (d, 1H).

### Example 50d

### (1S)-2-[1-(2,6-Dichloro-3-pyridyl)ethyl-methyl-amino]-1-(5-fluoro-2-pyridyl)ethanol

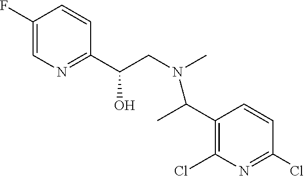

A mixture of (1S)-1-(5-fluoro-pyridin-2-yl)-2-methylamino-ethanol (Example 50c, 1.2 g, 7.06 mmol), 3-(1-bromo-ethyl)-2,6-dichloro-pyridine (Example 17d, 1.8 g, 7.06 mmol) and cesium carbonate (4.6 g, 14.1 mmol) in DMF (30 mL) was stirred overnight at room temperature. The mixture was diluted to 200 mL volume with ethyl acetate, filtered through a pad of Celite, and concentrated in vacuo. The residue was purified by flash column chromatography using a gradient of 30 to 50% ethyl acetate in hexane to afford 1.1 g (46%) of the title compound. ¹H NMR (400 MHz, CDCl₃) δ ppm 1.34 (m, 3H), 2.34 (m, 3H), 2.57 (m, 1H), 2.71 (m, 1H), 3.90 (m, 1H), 4.13 (m, 1H), 4.79 (m, 1H), 7.25 (m, 1H), 7.42 (m, 2H), 7.66 (m, 1H), 8.36 (m, 1H).

### Example 50e

### (2S)-8-Chloro-2-(5-fluoro-2-pyridyl)-4,5-dimethyl-3,5-dihydro-2H-pyrido[3,2-f][1,4]oxazepine

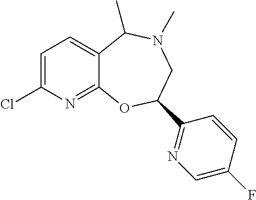

Sodium hydride (161 mg, 1.68 mmol, 95%) was added to a solution of (1S)-2-{[1-(2,6-dichloro-pyridin-3-yl)-ethyl]-methyl-amino}-1-(5-fluoro-pyridin-2-yl)-ethanol (Example 50d, 523 mg, 1.52 mmol) in THF (25 mL) at room temperature. The reaction mixture was heated at 45° C. overnight, cooled to room temperature and then saturated ammonium chloride and water were added. The mixture was extracted with ethyl acetate (2×100 mL) and the combined extracts were dried over anhydrous magnesium sulfate, filtered and concentrated in vacuo to afford 440 mg of the title 1 compound (94%) which was used in the next step without further purification. ¹H NMR (400 MHz, CDCl₃) δ ppm 1.53 (d, 3H), 3.25 (d, 1H), 3.46 (m, 1H), 3.58 (dd, 1H), 3.87 (q, 1H), 5.20 (m, 1H), 7.14 (m, 1H), 7.46 (m, 1H), 7.54 (m, 1H), 7.80 (m, 1H), 8.39 (m, 1H).

### Example 50f

### (2S)-2-(5-Fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine

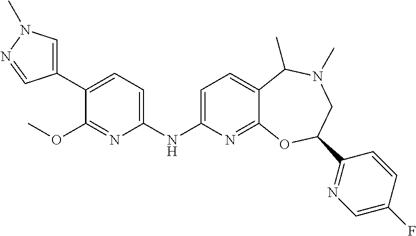

A mixture of (2S)-8-chloro-2-(5-fluoro-2-pyridyl)-4,5-dimethyl-3,5-dihydro-2H-pyrido[3,2-f][1,4]oxazepine (Example 50e, 300 mg, 0.98 mmol), 6-methoxy-5-(1-methyl-1H-pyrazol-4-yl)-pyridin-2-ylamine (Example 2a, 200 mg, 0.98 mmol) and cesium carbonate (414 mg, 1.27 mmol) in dioxane (15 mL) was degassed using nitrogen for 20 minutes. Palladium acetate (33 mg, 0.15 mmol) and Xantphos (169 mg, 0.30 mmol) were then added and the reaction mixture was heated in a microwave reactor at 145° C. for 1 hour. The mixture was cooled to room temperature, diluted with ethyl acetate, filtered through a plug of Celite and the filtrate was concentrated in vacuo. The residue was purified by flash column chromatography using a gradient of 2 to 5% methanol in DCM to afford 300 mg of the material which was further purified by preparative HPLC to yield 173 mg (37%) of the title compound. ¹H (400 MHz, CDCl₃) δ ppm 1.56 (m, 3H) 2.28 (s, 3H) 3.35 (m, 1H) 4.00 (m, 1H) 3.93 (s, 3H) 4.07 (s, 3H) 5.22 (m, 1H) 6.57 (t, 1H) 7.26 (s, 1H) 7.48 (m, 2H) 7.62 (m, 1H) 7.81 (m, 5H) 8.48 (s, 1H). ESMS m/z 476.2 [M+H]⁺.

### Example 51

### (2S)-2-(5-Fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine; isomer 2

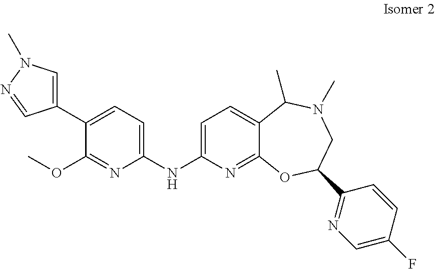

Chiral separation of (2S)-2-(5-fluoro-2-pyridyl)-N-[6-methoxy-5-(1-methylpyrazol-4-yl)-2-pyridyl]-4,5-dimethyl-3,5-dihydro-2H-pyrido[3,2-f][1,4]oxazepin-8-amine (Example 50f) as in Example 50 yielded 59 mg (43%) of isomer 2, the second isomer to elute.

### Example 52

### (−)-(2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine; isomer 1

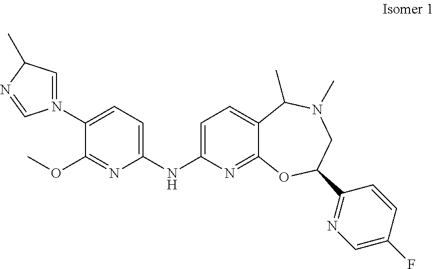

Chiral separation of (2S)-2-(5-fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine (Example 52a, 143 mg, 0.30 mmol) using SFC chromatography [Column: Chiralcel OD-H, 4.6*250 mm; 5 μm; Mobile phase: 20% MeOH; 80% CO₂; Flow: 50 mL/min] afforded 37 mg, (26%) of isomer 1, the first isomer to elute which had a negative optical rotation. ¹H NMR (500 MHz, DMSO-d₆) δ ppm 2.13 (s, 3H), 2.43 (s, 3H), 3.04 (d, 1H), 3.53 (dd, 1H), 3.84 (d, 1H), 3.95 (s, 3H), 5.17 (d, 1H), 6.95 (d, 1H), 7.06 (s, 1H), 7.64 (t, 2H), 7.69 (d, 1H), 7.76-7.80 (m, 2H), 7.80-7.86 (m, 1H), 8.57 (d, 1H), 9.93 (s, 1H). MS m/z 476.2 [M+H]⁺.

### Example 52a

### (2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine

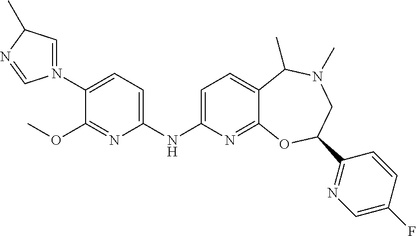

A mixture of (2S)-8-chloro-2-(5-fluoro-2-pyridyl)-4,5-dimethyl-3,5-dihydro-2H-pyrido[3,2-f][1,4]oxazepine (Example 50e, 300 mg, 0.98 mmol), 6-methoxy-5-(4-methylimidazol-1-yl)pyridin-2-ylamine (Example 6a, 200 mg, 0.98 mmol) and cesium carbonate (414 mg, 1.27 mmol) in dioxane (15 mL) was degassed for 20 minutes using nitrogen. Palladium acetate (33 mg, 0.15 mmol) and Xantphos (169 mg, 0.30 mmol) were added and the reaction mixture was heated in the microwave reactor at 145° C. for 1 hour. The mixture was cooled to room temperature, diluted with ethyl acetate, and filtered through a plug of Celite. The filtrate was concentrated in vacuo and the residue was purified by flash column chromatography (gradient elution, 2% to 8% methanol in dichloromethane) to yield 300 mg of a material which was further purified by preparative HPLC to afford 170 mg (36%) of the title compound. ¹H (400 MHz, CDCl₃) δ ppm 1.26 (m, 3H) 1.56 (d, 3H) 2.00 (m, 1H) 2.28 (s, 3H) 3.36 (m, 1H) 4.00 (s, 3H) 4.20 (m, 1H) 5.22 (m, 1H) 6.68 (m, 1H) 6.87 (s, 1H) 7.48 (m, 7H) 8.41 (br.s). ESMS m/z 476.2 [M+H]⁺.

### Example 53

### (−)-(2S)-2-(5-Fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine; isomer 2

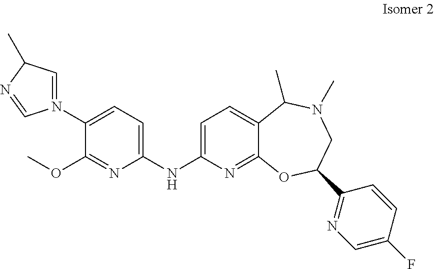

Chiral separation of (2S)-2-(5-fluoro-2-pyridyl)-N-[6-methoxy-5-(4-methylimidazol-1-yl)-2-pyridyl]-4,5-dimethyl-3,5-dihydro-2H-pyrido[3,2-f][1,4]oxazepin-8-amine as in Example 52 afforded 41 mg (29%) of isomer 2, the second isomer to elute, which had a negative optical rotation. ¹H NMR (500 MHz, DMSO-d₆) δ ppm 1.46 (d, 3H), 2.10-2.19 (m, 3H), 3.26 (dd, 1H), 3.34-3.41 (m, 1H), 3.96 (s, 3H), 4.29 (q, 1H), 5.12 (dd, 1H), 6.92 (d, 1H), 7.06 (s, 1H), 7.64 (d, 1H), 7.67 (d, 1H), 7.69 (d, 1H), 7.76 (dd, 1H), 7.83 (td, 1H), 7.90 (d, 1H), 8.56 (d, 1H), 9.96 (s, 1H). MS m/z 476.2 [M+H]⁺.

### Example 54

### (2S)—N-[3-Fluoro-6-methoxy-5-(1-methylpyrazol-4-yl)-2-pyridyl]-4-methyl-2-(4-methylthiazol-2-yl)-3,5-dihydro-2H-pyrido[3,2-f][1,4]oxazepin-8-amine

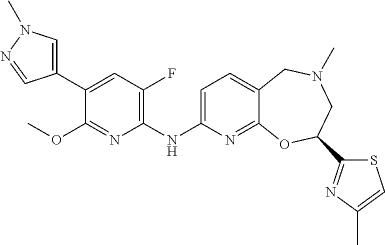

Palladium acetate (0.023 g, 15 mol %), Xantphos (0.117 g, 30 mol %) and cesium carbonate (0.23 g, 1.02 mmol) were added to a degassed mixture of (2S)-8-chloro-4-methyl-2-(4-methylthiazol-2-yl)-3,5-dihydro-2H-pyrido[3,2-f][1,4]oxazepine (Example 54e, 0.20 g, 0.68 mmol) and 3-fluoro-6-methoxy-5-(1-methyl-1H-pyrazol-4-yl)-pyridin-2-ylamine (Example 54f, 151 mg, 0.68 mmol) in anhydrous 1,4-dioxane (20 mL). The reaction mixture was purged with nitrogen for additional 20 minutes and then heated in a microwave reactor at 145° C. for 1 hour. The reaction mixture was diluted with dichloromethane (20 mL) and filtered through a small pad of Celite. The volatiles were removed under reduced pressure and the residue was purified by flash column chromatography using a gradient of 0% to 10% methanol in DCM and then further purified by preparative HPLC to obtain 96 mg (29%) of the title compound. ¹H NMR (400 MHz, CDCl₃) δ ppm 2.46 (s, 3H) 2.51 (s, 3H) 3.13 (dd, 2H) 3.47 (s, 2H) 3.56-3.77 (m, 2H) 3.84-4.00 (m, 7H) 5.36 (d, 1H) 6.54 (d, 1H) 6.76 (d, 1H) 6.91 (s, 1H) 7.16-7.33 (m, 1H) 7.43 (d, 1H) 8.07 (d, 1H). ESMS m/z 482.0 [M+H]⁺.

### Example 54a

### 2-Chloro-1-(4-methyl-thiazol-2-yl)-ethanone

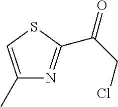

Butyl lithium (2.5 M, 24.2 mL, 0.06 mol) in diethyl ether was added to a solution of 4-methylthiazole (5.0 g, 0.05 mol) in anhydrous diethyl ether (100 mL) at −78° C. The reaction mixture was stirred at −78° C. for 30 minutes and N-(chloroacetyl)-morpholine (9.1 g, 0.055 mol) dissolved in anhydrous toluene (20 mL) was added. The reaction mixture was stirred at −78° C. for 1 hour, then quenched with saturated NaHCO₃ solution and extracted with ethyl acetate (2×100 mL). The combined extracts were dried over anhydrous MgSO₄, filtered and concentrated under reduced pressure. The residue was purified by flash column chromatography using a gradient of 0 to 30% ethyl acetate in hexane to afford 4.8 g (55%) of the title compound. ¹H NMR (400 MHz, CDCl₃) δ ppm 2.54 (s, 3H) 4.96 (s, 2H) 7.34 (s, 1H). ESMS m/z 175.9 [M+H]⁺.

### Example 54b

### (1R)-2-Chloro-1-(4-methylthiazol-2-yl)ethanol

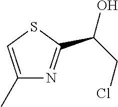

To a solution of the freshly prepared “Noyoris Catalyst” (Angew. Chem. Int. Eng. 1997, 36, 285-288; 546 mg, 20 mol %,) in anhydrous DMF, 2-chloro-1-(4-methyl-thiazol-2-yl)-ethanone (Example 54a, 4.8 g, 27.3 mmol) followed by a mixture of formic acid and triethylamine (5:2, 10 mL) were added at 0° C. The reaction mixture was then stirred at room temperature for 2 hours, methanol (2.5 mL) was added and stirring was continued for 5 minutes. The volatiles were removed under reduced pressure and the residue was taken in diethyl ether-dichloromethane mixture (4:1, 200 mL). The organic phase was washed with saturated NaHCO₃ solution (150 mL), brine, dried over anhydrous MgSO₄, filtered and concentrated under reduced pressure. The residue was purified by flash column chromatography using a gradient of 0% to 5% methanol in dichloromethane to afford the title compound (3.7 g, 76%). ¹H NMR (400 MHz, CDCl₃) δ ppm 2.44 (s, 3H) 3.24 (d, 1H) 3.85 (dd, 1 H) 4.03 (dd, 1H) 5.17 (ddd, 1H) 6.90 (s, 1H). ESMS m/z 178.0 [M+H].

### Example 54c

### (1S)-2-(Methylamino)-1-(4-methylthiazol-2-yl)ethanol

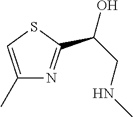

A solution of methylamine in ethanol (35%, 20 mL) was added to a solution of (1R)-2-chloro-1-(4-methylthiazol-2-yl)ethanol (Example 54b, 3.7 g, 20.8 mmol) in anhydrous ethanol (200 mL). The reaction mixture was heated at 65° C. for 48 hours, then cooled to room temperature and concentrated under reduced pressure. The residue was purified by flash column chromatography using a gradient of 0% to 5% methanol in dichloromethane to afford the title compound (3.0 g, 84%). ¹H NMR (400 MHz, CDCl₃) δ ppm 2.37 (s, 3H) 2.87 (s, 3H) 3.40 (dd, 1H) 3.61 (dd, 1H) 5.62-5.73 (m, 1H) 6.90 (s, 1H). ESMS m/z 173.0 [M+H]⁺.

### Example 54d

### (1S)-2-[(2,6-Dichloro-3-pyridyl)methyl-methyl-amino]-1-(4-methylthiazol-2-yl)ethanol

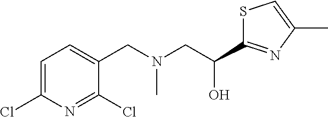

(1S)-2-(Methylamino)-1-(4-methylthiazol-2-yl)ethanol (Example 54c, 3.0 g, 17.4 mmol) was added to a mixture of cesium carbonate (11.35 g, 34.8 mmol) and 3-bromomethyl-2,6-dichloro-pyridine ((CAS 58596-59-1, 4.2 g, 17.4 mmol) in anhydrous DMF (100 mL) at room temperature under a nitrogen atmosphere. The reaction mixture was stirred overnight, filtered and concentrated under reduced pressure. The residue was purified by flash column chromatography using a gradient of 0% to 5% methanol in dichloromethane to afford the title compound (2.0 g, 35%). ¹H NMR (400 MHz, CDCl₃) δ ppm 2.33 (s, 3H) 2.43 (s, 3H) 2.74-2.85 (m, 1H) 2.97-3.06 (m, 1H) 3.58-3.72 (m, 1 H) 3.72-3.86 (m, 1H) 3.97 (br. s., 1H) 5.07 (dd, 1H) 6.84 (s, 1H) 7.24-7.34 (m, 1H) 7.71 (d, 1 H). ESMS m/z 333.9 [M+H]⁺.

### Example 54e

### (2S)-8-Chloro-4-methyl-2-(4-methylthiazol-2-yl)-3,5-dihydro-2H-pyrido[3,2-f][1,4]oxazepine

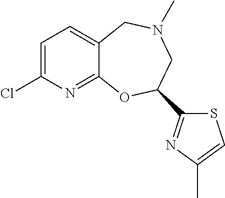

Sodium hydride (95% powder, 0.193 g, 7.22 mmol) was added to a solution of (1S)-2-[(2,6-dichloro-3-pyridyl)methyl-methyl-amino]-1-(4-methylthiazol-2-yl)ethanol (Example 54d, 2.0 g, 6.02 mmol) in anhydrous THF (100 mL) at −78° C. The reaction mixture was allowed to warm at room temperature and stirred overnight. The reaction was quenched with saturated NH₄Cl solution (5.0 mL) and concentrated under reduced pressure. The residue was taken in ethyl acetate (100 mL) and the organic phase was washed with water and brine, dried over Na₂SO₄, and concentrated under reduced pressure. The residue was purified by flash column chromatography using a gradient of 0 to 7% methanol in dichloromethane to afford 0.8 g (45%) of the title compound. ¹H NMR (400 MHz, CDCl₃) δ ppm 2.45 (s, 4H) 2.49 (s, 3H) 3.18 (dd, 1H) 3.60 (d, 1H) 3.70 (d, 1H) 3.95 (d, 1H) 5.35 (dd, 1H) 6.92 (s, 1H) 7.12 (d, 1H) 7.54 (d, 1H). ESMS m/z 296.0 [M+H]⁺.

### Example 54f

### 3-Fluoro-6-methoxy-5-(1-methyl-1H-pyrazol-4-yl)-pyridin-2-ylamine

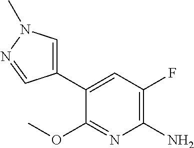

A mixture of 4-bromo-2-fluoro-5-methoxy-phenylamine (1.54 g, 7.0 mmol, prepared as described in: U.S. Pat. No. 4,818,276), 1-methyl-4-(4,4,5,5-tetramethyl-1,3,2-dioxaborolan-2yl)-1H-pyrazole (2.18 g, 10.5 mmol) and potassium carbonate (1.45 g, 10.5 mmol) in a mixture of dimethoxyethane, ethanol and water (7:2:1, 20 mL) was purged with nitrogen for 15 minutes. [1,1′-Bis(di-tert-butylphosphino)-ferrocene]palladium(II) dichloride (137 mg, 0.21 mmol) was added and the reaction mixture was heated in a sealed tube at 90° C. overnight. The mixture was cooled to room temperature, diluted with ethyl acetate (50 mL) and filtered. The organic phase was washed with water, brine, dried over sodium sulfate, and concentrated under reduced pressure. The residue was purified by flash column chromatography using a gradient of 0.5 to 2% methanol in DCM to afford 970 mg (63%) of the title compound. ¹H NMR (400 MHz, CDCl₃) δ ppm 3.71 (br. s., 2H) 3.82 (s, 3H) 3.92 (s, 3H) 6.38 (d, 1H) 7.13 (d, 1H) 7.71 (s, 1H) 7.74 (s, 1H). ESMS m/z 222.0 [M+1]⁺.

### Example 55

### 3-(4-Methylimidazol-1-yl)-6-[[(2S)-4-methyl-2-(4-methylthiazol-2-yl)-3,5-dihydro-2H-pyrido[3,2-f][1,4]oxazepin-8-yl]amino]pyridine-2-carbonitrile

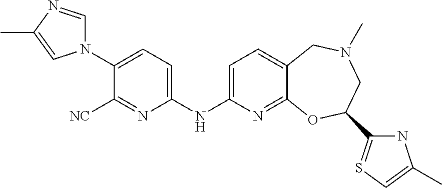

6-Amino-3-(4-methyl-imidazol-1-yl)-pyridine-2-carbonitrile (Example 48c, 146 mg, 0.74 mmol) was added to a solution of (2S)-8-chloro-4-methyl-2-(4-methylthiazol-2-yl)-3,5-dihydro-2H-pyrido[3,2-f][1,4]oxazepine (Example 54e, 200 mg, 0.68 mmol) in anhydrous 1,4-dioxane (12 mL) and the mixture was purged with nitrogen for 20 minutes. Palladium acetate (24 mg, 0.10 mmol), Xantphos (117 mg, 0.20 mmol) and cesium carbonate (286 mg, 0.88 mmol) were added and the reaction mixture was degassed for additional 10 minutes. The reaction mixture was heated in a microwave reactor at 145° C. for 1 hour, then cooled to room temperature and concentrated under reduced pressure. The residue was purified by flash chromatography using a gradient of 0 to 5% methanol in dichloromethane to afford 151 mg of the material which was further purified by preparative HPLC to afford the title compound (61.7 mg 20%). ¹H NMR (400 MHz, CD₃OD) δ ppm 2.27 (s, 3H) 2.43 (s, 3H) 2.51 (s, 3H) 3.12 (dd, 1H) 3.56 (d, 1H) 3.73-3.81 (m, 1H) 3.83-3.92 (m, 1H) 5.34 (d, 1H) 7.18 (d, 2H) 7.50 (d, 1H) 7.71 (d, 1H) 7.84 (d, 1H) 7.92 (s, 1H) 8.08 (d, 1H). ESMS m/z 457 [M−1]⁻.

### Example 56

### (2R)—N-[6-Methoxy-5-(1H-pyrazol-4-yl)-2-pyridyl]-4-methyl-2-phenyl-3,5-dihydro-2H-pyrido[3,2-f][1,4]oxazepin-8-amine

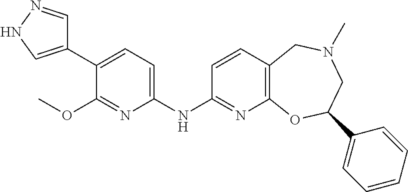

TFA (1% in DCM, 10 mL) was added to an ice-cold solution of tert-butyl 4-[2-methoxy-6-[[(2R)-4-methyl-2-phenyl-3,5-dihydro-2H-pyrido[3,2-f][1,4]oxazepin-8-yl]amino]-3-pyridyl]pyrazole-1-carboxylate (Example 56b, 0.11 g, 0.208 mmol) in anhydrous DCM (10 mL) under a nitrogen atmosphere. The reaction mixture was allowed to warm to room temperature, stirred overnight and then neutralized using saturated NaHCO₃ solution (5.0 mL). The organic phase was separated, dried over anhydrous MgSO₄ and concentrated in vacuo to afford 64 mg (72%) of the title compound. ¹H NMR (400 MHz, CDCl₃) δ ppm 2.49 (s, 3H) 3.10 (d, 2H) 3.49 (s, 1H) 3.69 (s, 1H) 3.88 (d, 1H) 4.07 (s, 3H) 5.11 (s, 1H) 6.58 (d, 1H) 7.22 (br. s., 1H) 7.33 (d, 1H) 7.39 (t, 2H) 7.43-7.49 (m, 2 H) 7.53 (d, 1H) 7.70 (d, 1H) 7.77 (d, 1H) 7.96 (s, 2H). ESMS m/z 429.1 [M+H]⁺.

### Example 56a

### tert-Butyl 4-(6-amino-2-methoxy-3-pyridyl)pyrazole-1-carboxylate

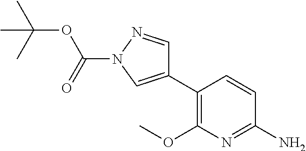

PdCl₂(PPh₃)₂ (0.18 g, 0.25 mmol) was added to a degassed mixture of 5-bromo-6-methoxy-pyridin-2-ylamine (Example 7b, 1.0 g, 4.02 mmol), tert-butyl 4-(4,4,5,5-tetramethyl-1,3,2-dioxaborolan-2-yl)pyrazole-1-carboxylate (CAS 552846-17-0, 2.17 g, 7.39 mmol) and K₂CO₃ (1.02 g, 7.39 mmol) in a mixture of DME and water (6:2, 140 mL). The reaction mixture was heated in a sealed tube at 90° C. overnight, cooled to room temperature, diluted with ethyl acetate and filtered. The filtrate was concentrated in vacuo and the residue was purified by flash column chromatography using a gradient of 0 to 50% EtOAc in hexane to afford 1.2 g (84%) of the title compound. ¹H NMR (400 MHz, CDCl₃) δ ppm 1.24 (s, 9H) 3.97 (s, 3H) 4.36 (br. s., 2H) 6.14 (d, 1H) 7.59 (d, 1H) 8.01 (s, 1H) 8.37 (s, 1H).

### Example 56b

### tert-Butyl 4-[2-methoxy-6-[[(2R)-4-methyl-2-phenyl-3,5-dihydro-2H-pyrido[3,2-f][1,4]oxazepin-8-yl]amino]-3-pyridyl]pyrazole-1-carboxylate

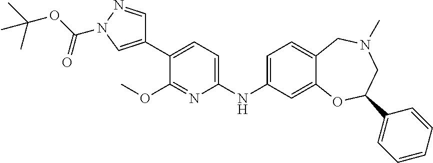

Palladium acetate (0.023 g, 5 mol %), Xantphos (0.118 g, 30 mol %) and cesium carbonate (0.33 g, 1.02 mmol) were added to a degassed mixture of (R)-8-Chloro-4-methyl-2-phenyl-2,3,4,5-tetrahydropyrido[3,2-f][1,4]oxazepine (Example 14c, 0.26 g, 0.946 mmol) and tert-butyl 4-(6-amino-2-methoxy-3-pyridyl)pyrazole-1-carboxylate (Example 56a, 0.280 g, 0.946 mmol) in anhydrous 1,4-dioxane (20 mL). The reaction mixture was purged with nitrogen for an additional 20 minutes and then heated in a microwave reactor at 145° C. for 1 hour. The reaction mixture was diluted with dichloromethane (20 mL) and filtered through a small pad of Celite and concentrated under reduced pressure. The residue was purified by flash column chromatography using a gradient of 0% to 10% methanol in DCM, followed by prep HPLC to afford 120 mg (25%) of the title compound. ¹H NMR (400 MHz, CDCl₃) δ ppm 1.68 (s, 9H) 2.50 (s, 3H) 3.11 (d, 1H) 3.12 (s, 1 H) 3.64-3.77 (m, 1H) 3.89 (d, 1H) 4.08 (s, 3H) 5.07-5.19 (m, 1H) 6.59 (d, 1H) 7.33 (d, 1H) 7.39 (t, 2H) 7.44-7.50 (m, 2H) 7.54 (d, 2H) 7.70 (d, 1H) 7.81 (d, 1H) 8.05 (s, 1H) 8.43 (s, 1H). ESMS m/z 529.2 [M+H]⁺.

### Example 57

### (+)-N-[6-Methoxy-5-(1-methylpyrazol-4-yl)-2-pyridyl]-4-methyl-2-(3,3,3-trifluoropropyl)-3,5-dihydro-2H-pyrido[3,2-f][1,4]oxazepin-8-amine; isomer 1

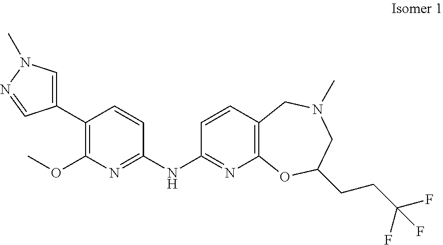

Separation of the enantiomers of N-[6-methoxy-5-(1-methylpyrazol-4-yl)-2-pyridyl]-4-methyl-2-(3,3,3-trifluoropropyl)-3,5-dihydro-2H-pyrido[3,2-f][1,4]oxazepin-8-amine (Example 27) by SFC chromatography [Column: Chiralcel OD-H; (4.6*250 mm; 5 μm) Mobile phase: 20% MeOH; 80% CO2; Flow: 50 mL/min] yielded 42 mg (27%) of isomer 1 (the first isomer to elute) which has a positive optical rotation. ¹H NMR (500 MHz, DMSO-d₆) δ ppm 1.71-1.83 (m, 2H) 2.30 (s, 3H) 2.55-2.67 (m, 2H) 2.69-2.77 (m, 1H) 2.90-2.96 (m, 1H) 3.56 (s, 2H) 3.86 (s, 3H) 4.00 (s, 3H) 4.01-4.08 (m, 1H) 7.01-7.04 (m, 1H) 7.54-7.58 (m, 1H) 7.69-7.73 (m, 1H) 7.84 (dd, 2H) 8.02 (s, 1H) 9.59 (s, 1H). MS (ES+) m/z 463.2 [M+H]⁺.

### Example 58

### (−)-N-[6-Methoxy-5-(1-methylpyrazol-4-yl)-2-pyridyl]-4-methyl-2-(3,3,3-trifluoropropyl)-3,5-dihydro-2H-pyrido[3,2-f][1,4]oxazepin-8-amine; isomer 2

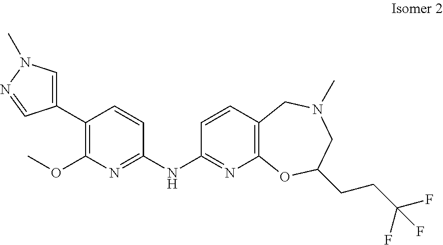

Separation of the enantiomers of N-[6-methoxy-5-(1-methylpyrazol-4-yl)-2-pyridyl]-4-methyl-2-(3,3,3-trifluoropropyl)-3,5-dihydro-2H-pyrido[3,2-f][1,4]oxazepin-8-amine (Example 27) as in Example 57 yielded 38 mg (25%) of isomer 2 (the second isomer to elute) which has a negative optical rotation. ¹H NMR (500 MHz, DMSO-d₆) δ ppm 1.71-1.83 (m, 2H) 2.30 (s, 3H) 2.55-2.67 (m, 2H) 2.69-2.77 (m, 1H) 2.90-2.96 (m, 1H) 3.56 (s, 2H) 3.86 (s, 3H) 4.00 (s, 3H) 4.01-4.08 (m, 1H) 7.01-7.04 (m, 1H) 7.54-7.58 (m, 1H) 7.69-7.73 (m, 1H) 7.84 (dd, 2H) 8.02 (s, 1 H) 9.59 (s, 1H). MS (ES+) m/z 463.2 [M+H]⁺.

### Example 59

### (−)-N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine; isomer 1

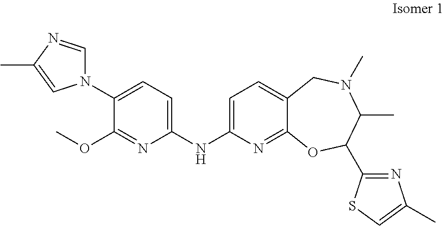

Separation of the isomers of N-(6-methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine (Example 59a 120 mg, 0.25 mmol) by SFC chromatography [Column: Chiralpak OD-H; (21.2*250 mm) Mobile phase: 30% IPA+0.1% DEA; 80% CO2, Flow: 50 ml/min] yielded isomer 1 (18 mg, 15%), the first isomer to elute which has a negative optical rotation. ¹H NMR (500 MHz, DMSO-d₆) δ ppm 1.71-1.83 (m, 2H) 2.30 (s, 3H) 2.55-2.67 (m, 2H) 2.69-2.77 (m, 1H) 2.90-2.96 (m, 1H) 3.56 (s, 2H) 3.86 (s, 3H) 4.00 (s, 3H) 4.01-4.08 (m, 1H) 7.01-7.04 (m, 1H) 7.54-7.58 (m, 1H) 7.69-7.73 (m, 1H) 7.84 (dd, 2H) 8.02 (s, 1H) 9.59 (s, 1H). MS (ES+) m/z 478 [M+H]⁺

### Example 59a

### N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine

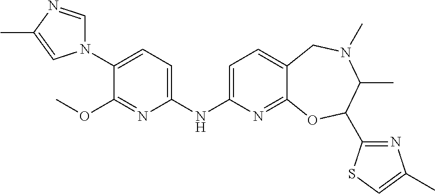

To 8-chloro-3,4-dimethyl-2-(4-methylthiazol-2-yl)-2,3,4,5-tetrahydropyrido[3,2-f][1,4]oxazepine (Example 29e, 0.130 g, 0.42 mmol) in DME (3 mL) were 6-methoxy-5-(4-methyl-1H-imidazol-1-yl)pyridin-2-ylamine (Example 6a, 86 mg, 0.42 mmol), cesium carbonate (0.205 g, 0.63 mmol), 2-(dicyclohexylphosphino)biphenyl (15 mg, 0.04 mmol) and palladium acetate (9.42 mg, 0.04 mmol) added. The reaction was heated to 110° C. for 60 min under argon atmosphere. The reaction mixture was filtered through celite, washed with DCM and the solvents were evaporated. The crude product was purified by silica flash chromatography, MeOH, 0-5%, in DCM yielding N-(6-methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine (120 mg, 60%). MS (ES+) m/z 478 [M+H]

### Example 60

### (+)-N-(6-Methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine; isomer 2

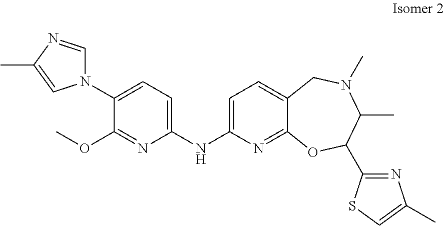

Separation of the isomers as in Example 59 yielded N-(6-methoxy-5-(4-methyl-1H-imidazol-1-yl)pyridin-2-yl)-3,4-dimethyl-2-(4-methylthiazol-2-yl)-2,3,4,5-tetrahydropyrido[3,2-f][1,4]oxazepin-8-amine isomer 2 (31 mg, 26%), the second isomer to elute, which has a positive optical rotation. ¹H NMR (500 MHz, DMSO-d₆) δ ppm 1.71-1.83 (m, 2H) 2.30 (s, 3H) 2.55-2.67 (m, 2H) 2.69-2.77 (m, 1H) 2.90-2.96 (m, 1H) 3.56 (s, 2H) 3.86 (s, 3H) 4.00 (s, 3H) 4.01-4.08 (m, 1H) 7.01-7.04 (m, 1H) 7.54-7.58 (m, 1H) 7.69-7.73 (m, 1H) 7.84 (dd, 2H) 8.02 (s, 1 H) 9.59 (s, 1H). MS (ES+) m/z 478 [M+H]⁺.

### Example 61

### (+)-[6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine; isomer 1

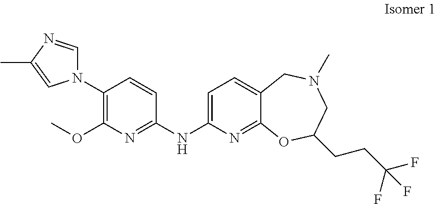

Chiral separation of the enantiomers of [6-methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine (Example 28, 167 mg, 0.36 mmol) by SFC chromatography [Column: Chiralcel OD-H, 4.6*250 mm; 5 μm; Mobile phase: 20% MeOH; 80% CO₂; Flow: 50 mL/min] yielded isomer 1 (59 mg, 35%), the first isomer to elute which has a positive optical rotation. ¹H NMR (500 MHz, DMSO-d₆) δ ppm 1.71-1.83 (m, 2H) 2.14 (d, 3H) 2.30 (s, 3H) 2.55-2.66 (m, 2H) 2.69-2.78 (m, 1H) 2.89-2.96 (m, 1H) 3.57 (s, 2H) 3.93 (s, 3H) 4.01-4.09 (m, 1H) 7.06-7.09 (m, 2H) 7.57-7.72 (m, 4 H) 9.82 (s, 1H). MS (ES+) m/z 463.2 [M+H]⁺.

### Example 62

### (−)-[6-Methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine; isomer 2

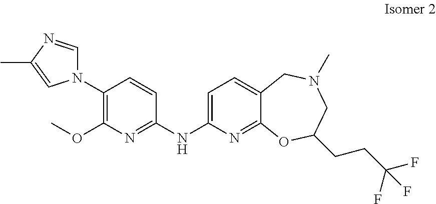

Separation of the enantiomers of Example 28 as in Example 61 yielded [6-methoxy-5-(4-methyl-imidazol-1-yl)-pyridin-2-yl]-[6-methyl-8-(3,3,3-trifluoro-propyl)-5,6,7,8-tetrahydro-9-oxa-1,6-diaza-benzocyclohepten-2-yl]-amine isomer 2 (57 mg, 34%), the second isomer to elute, which has a negative optical rotation. ¹H NMR (500 MHz, DMSO-d₆) δ ppm 1.70-1.83 (m, 2H) 2.14 (d, 3 H) 2.30 (s, 3H) 2.55-2.66 (m, 2H) 2.70-2.78 (m, 1H) 2.89-2.96 (m, 1H) 3.57 (s, 2H) 3.93 (s, 3 H) 4.02-4.09 (m, 1H) 7.06-7.09 (m, 2H) 7.57-7.66 (m, 2H) 7.66-7.71 (m, 2H) 9.80-9.83 (m, 1H). MS (ES+) m/z 463.2 [M+H]⁺.

### Example 63

### N-[6-Methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine; isomer 1

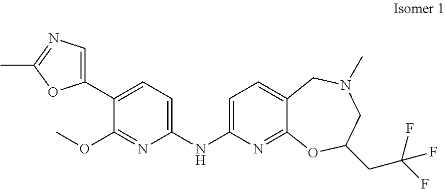

Chiral separation of the enantiomers of N-[6-methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine (Example 47, 224 mg, 0.50 mmol) by SFC chromatography [Column: Chiralcel OD-H, 4.6*250 mm; 5 μm; Mobile phase: 20% MeOH; 80% CO₂; Flow: 50 mL/min] yielded isomer 1 (66 mg, 59%), the first isomer to elute. ¹H NMR (500 MHz, DMSO-d₆) δ ppm 2.30 (s, 3H) 2.45 (s, 3H) 2.59-2.77 (m, 2H) 2.83 (dd, 1H) 2.90-2.98 (m, 1H) 3.61 (s, 2H) 4.04 (s, 3H) 4.26-4.34 (m, 1H) 7.21 (s, 1H) 7.26 (d, 1 H) 7.56-7.65 (m, 2H) 7.82 (d, 1H) 9.85 (s, 1H). MS (ES+) m/z 450.6 [M+H]⁺.

### Example 64

### N-[6-Methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine; isomer 2

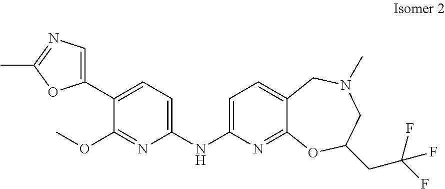

Separation of the enantiomers of Example 47 as in Example 63 yielded N-[6-methoxy-5-(2-methyloxazol-5-yl)-2-pyridyl]-4-methyl-2-(2,2,2-trifluoroethyl)-3,5-dihydro-2H-pyrido[3,2-f][1,4]oxazepin-8-amine isomer 2 (65 mg, 58%), the second isomer to elute. ¹H NMR (500 MHz, DMSO-d₆) δ ppm 2.30 (s, 3H) 2.45 (s, 3H) 2.59-2.77 (m, 2H) 2.83 (dd, 1H) 2.90-2.98 (m, 1H) 3.61 (s, 2H) 4.04 (s, 3H) 4.26-4.34 (m, 1H) 7.21 (s, 1H) 7.26 (d, 1H) 7.56-7.65 (m, 2H) 7.82 (d, 1H) 9.85 (s, 1H). MS (ES+) m/z 450.5 [M+H]⁺.

### Example 65

### (−)-N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine; isomer 1

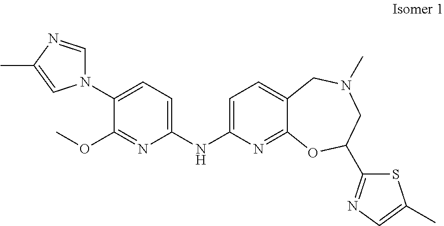

Chiral separation of the enantiomers of N-(6-methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine (Example 40, 127 mg, 0.27 mmol) by SFC chromatography [Column: Chiralcel AD-H, 4.6*250 mm; 5 μm; Mobile phase: (25% EtOH+0.1% DEA); 80% CO₂; Flow: 50 mL/min] yielded isomer 1 (46 mg, 36%), the first isomer to elute which has a negative optical rotation. ¹H NMR (500 MHz, DMSO-d₆) δ ppm 2.14 (d, 3H) 2.37 (s, 3H) 2.46 (d, 3H) 3.02 (dd, 1H) 3.43 (d, 1H) 3.65-3.79 (m, 2H) 3.95 (s, 3H) 5.32 (d, 1H) 7.05-7.10 (m, 2H) 7.49 (d, 1H) 7.65-7.68 (m, 2H) 7.71 (d, 1H) 7.73-7.77 (m, 1H) 9.95 (s, 1H). MS (ES+) m/z 464.2 [M+H]⁺.

### Example 66

### (+)-N-(6-Methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine, isomer 2

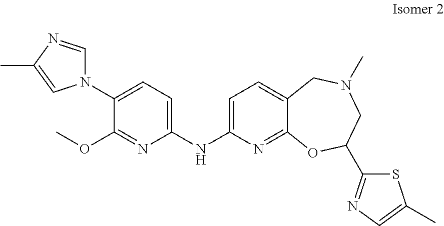

Separation of the enantiomers of Example 40, (127 mg, 0.27 mmol)) as in Example 65 yielded N-(6-s methoxy-5-(4-methyl-1H-imidazol-1-yl)pyridin-2-yl)-4-methyl-2-(5-methylthiazol-2-yl)-2,3,4,5-tetrahydropyrido[3,2-f][1,4]oxazepin-8-amine isomer 2 (43 mg, 34%), the second isomer to elute which has a positive optical rotation. ¹H NMR (500 MHz, DMSO-d₆) δ ppm 2.14 (d, 3H) 2.37 (s, 3 H) 2.46 (d, 3H) 3.02 (dd, 1H) 3.43 (d, 1H) 3.65-3.79 (m, 2H) 3.95 (s, 3H) 5.32 (d, 1H) 7.05-7.10 (m, 2H) 7.49 (d, 1H) 7.65-7.68 (m, 2H) 7.71 (d, 1H) 7.73-7.77 (m, 1H) 9.95 (s, 1H). MS (ES+) m/z 464.2 (M+H)⁺.

### Example 67

### (+)-[8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine; isomer 1

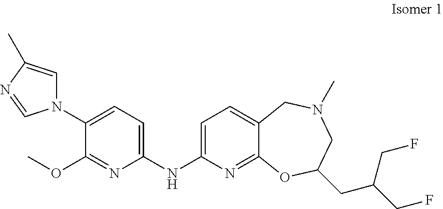

Chiral separation of the enantiomers of [8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine (Example 32, Preparation I; 150 mg, 0.33 mmol) by SFC chromatography [Column: Chiralcel OJ-H; (4.6*250 mm; 5 μm); Mobile phase: 20% MeOH; 80% CO₂; Flow: 50 mL/min] yielded isomer 1 (49 mg, 33%), the first isomer to elute which has a positive optical rotation. ¹H NMR (500 MHz, DMSO-d₆) δ ppm 1.52 (ddd, 1H), 1.65-1.75 (m, 1H), 2.14 (s, 3H), 2.30 (s, 3 H), 2.71 (dd, 1H), 2.90 (d, 1H), 3.58 (s, 2H), 3.93 (s, 3H), 4.07-4.16 (m, 1H), 4.48-4.55 (m, 1 H), 4.55-4.64 (m, 2H), 4.64-4.72 (m, 1H), 7.07 (s, 1H), 7.20 (d, 1H), 7.57 (d, 2H), 7.63 (d, 1 H), 7.70 (d, 1H), 9.78 (s, 1H). MS (ES+) m/z 459.2 [M+H]⁺.

### Example 68

### (−)-[8-(3-Fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine; isomer 2

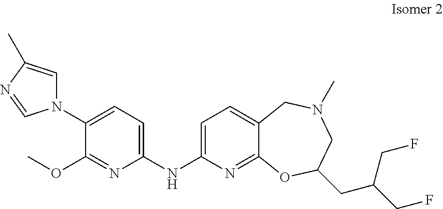

Separation of the enantiomers of Example 32 (Preparation I; 127 mg, 0.27 mmol)) as in Example 67 yielded [8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine, isomer 2 (49 mg, 33%), the second isomer to elute which has a negative optical rotation. ¹H NMR (500 MHz, DMSO-d₆) δ ppm 1.52 (ddd, 1H), 1.65-1.75 (m, 1H), 2.14 (s, 3H), 2.30 (s, 3H), 2.71 (dd, 1H), 2.90 (d, 1H), 3.58 (s, 2H), 3.93 (s, 3H), 4.07-4.16 (m, 1H), 4.48-4.55 (m, 1H), 4.55-4.64 (m, 2H), 4.64-4.72 (m, 1H), 7.07 (s, 1H), 7.20 (d, 1H), 7.57 (d, 2H), 7.63 (d, 1H), 7.70 (d, 1H), 9.78 (s, 1H). MS (ES+) m/z 459.2 [M+H]⁺.

### Biological Assays

The level of activity of the compounds on Aβ formation was tested using the following method:

Compounds were diluted in 100% DMSO and stored at 20° C. prior to use. Human Embryonic Kidney (HEK) cell line stably expressing APP with the Swedish mutation (APPswe) were cultured using Dulbecco's Modified Eagles medium (DMEM) supplied with 4500 g/l glucose, Na-pyruvate and GlutaMAX with 10% FBS, 100 U/ml penicillin-streptomycin (PEST) respectively, 1× non-essential amino acids (NEAA), 10 μM Hepes, 100 μg/ml Zeocine. Cells at about 80% confluence were washed with PBS, detached from culture flasks using 1× Trypsin/EDTA diluted in PBS, re-suspended in cell media and plated in 384-well poly-d-lysine coated cell culture plates at about 10000-15000 cells/well, in 25 μL cell media. Optionally, cryo-preserved cells (frozen and stored at −140° C. in 90% cell media and 10% DMSO) were thawed, washed and plated as above. Next the cells were incubated for 15-24 h at 37° C. and 5% CO₂, after which cell medium was changed. Fresh medium containing test compound diluted ×200 from prepared compound plate was added to the cells before further incubation for 4-6 hours at 37° C. and 5% CO₂. After incubation with test compound the amount of Aβ peptides, including Aβ42, Aβ40, Aβ39, Aβ38 and Aβ37, as well as total Aβ levels, secreted to cell medium was analyzed using the electrochemiluminescence assay technology from Meso Scale Discovery (MSD), in combination with specific antibodies raised against the different Aβ peptides. Aβ42, Aβ40 and Aβ38 antibodies were purchased from MSD and Aβ37 and Aβ39 were made in house. Total Aβ was measured using a combination of 6E10 and 4G8 anti Aβ antibodies from MSD. Potential cytotoxic effects of the compounds were usually assayed by measuring the ATP content (ViaLight) from cell lysate.

The level of activity of the compounds on release and translocation of Notch intracellular domain into the cell nucleus was tested using the following method:

Compounds were diluted in 100% DMSO and stored at 20° C. prior to use. Human Embryonic Kidney cell line overexpressing Notchl-E-human-myc construct (referred to as HEK/AENotch) were cultured in T75 cm² or T225 cm² cell culture flasks using Dulbecco's Modified Eagles medium (DMEM) supplemented with 4500 g/l glucose, Na-pyruvate and GlutaMAX with 10% FBS, 100 U/ml penicillin-streptomycin (PEST) respectively, 1× non-essential amino acids (NEAA), 10 μM Hepes and 100 μg/ml Hygromycin. Cells at about 80% confluence were washed with PBS, detached from the flask bottom using 1× Trypsin/EDTA diluted in PBS, re-suspended in cell media and plated in 384-well plates at about 10000 cells/well, in 25 μL cell media. Optionally, cryo-preserved cells (frozen and stored at −140° C. in 90% cell media and 10% DMSO) were thawed, washed and plated as above. Next the cells were incubated for 15-24 h at 37° C. and 5% CO₂, after which cell medium was replaced with fresh medium containing 3 μM Lactacystin and test compound diluted ×200 from prepared compound plate giving 0.5% final DMSO concentration, and the highest compound concentration typically 25 μM. Cells were then incubated 4-6 h with test compound before being fixed, washed and immunostained with NICD antibody (rabbit Notch 1 (C-20)). Image acquisition of cytosolic/membrane and nuclear fluorescence as well as subsequent analysis of the nucleus and cytosolic fluorescence ratio was carried out using ImageXpress. Potential cytotoxic effects of the compounds were usually assayed by analysis of nucleus area or cell number.

### Results

Typical IC₅₀ values of Aβ42 release for the compounds of the present invention are in the range of about 1 to about 1000 nM. Typical IC₅₀ values of Aβ total and Notch release are in the range of about 500 to about 50000 nM. Biological data on exemplified compounds are given below in Table 1.

TABLE 1
IC₅₀ values of Aβ42, Aβtotal and Notch release
for the compounds of the present invention.

| Example | IC₅₀ Aβ42 | IC₅₀ Aβtotal | IC₅₀ Notch |
|---|---|---|---|
| number | (nM) | (nM) | (nM) |
| 1 | 225 | | >50000 |
| 2 | 23 | | 1140 |
| 3 | 17 | | >25000 |
| 4 | 9 | 512 | >25000 |
| 5 | 21 | | >25000 |
| 6 | 18 | | >7900 |
| 7 | 135 | | >25000 |
| 8 | 5 | | 3250 |
| 9 | 19 | | >15800 |
| 10 | 226 | | |
| 11 | 14 | | 1440 |
| 12 | 13 | 1852 | >25000 |
| 13 | 48 | | >5000 |
| 14 | 24 | | >50000 |
| 15 | 137 | | |
| 16 | 305 | | |
| 17 | 14 | | >7900 |
| 18 | 37 | | >7900 |
| 19 | 124 | | |
| 20 | 91 | | >7900 |
| 21 | 179 | | |
| 22 | 10 | | 1680 |
| 23 | 55 | | >2500 |
| 24 | 81 | | >7900 |
| 25 | 73 | | >7900 |
| 26 | 56 | | >7900 |
| 27 | 12 | 8724 | 16300 |
| 28 | 7 | >1000 | >25000 |
| 29 | 68 | | 5000 |
| 30 | 7 | | >25000 |
| 31 | 22 | 9472 | >7900 |
| 32 | 7 | >5000 | >7900 |
| 33 | 8 | >5000 | >25000 |
| 34 | 16 | | >25000 |
| 35 | 12 | | >25000 |
| 36 | 12 | | >25000 |
| 37 | 4 | >1000 | >25000 |
| 38 | 15 | | >25000 |
| 39 | 28 | | >25000 |
| 40 | 9 | | 3640 |
| 41 | 60 | | >25000 |
| 42 | 46 | | >25000 |
| 43 | 493 | | |
| 44 | 374 | | |
| 45 | 38 | | |
| 46 | 33 | | >25000 |
| 47 | 4 | | >25000 |
| 48 | 61 | | >25000 |
| 49 | 2 | 5461 | 2170 |
| 50 | 60 | | 2310 |
| 51 | 19 | | >25000 |
| 52 | 10 | | 7200 |
| 53 | 11 | | >7900 |
| 54 | 18 | | 6490 |
| 55 | 44 | | >25000 |
| 56 | 98 | | >25000 |
| 57 | 5 | | >11500 |
| 58 | 18 | | >25000 |
| 59 | 7 | | >25000 |
| 60 | 17 | | >25000 |
| 61 | 3 | >1000 | >25000 |
| 62 | 5 | | >25000 |
| 63 | 3 | | |
| 64 | 4 | | |
| 65 | 9 | | >25000 |
| 66 | 38 | | >25000 |
| 67 | 10 | >25000 | 6230 |
| 68 | 9 | | >25000 |

1-19. (canceled)

20. A compound that is [8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine having the following formula: 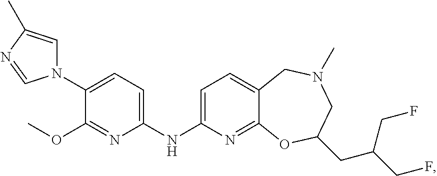
or a pharmaceutically acceptable salt thereof.

21. A compound according to claim 20 which is an isomer thereof having a positive optical rotation that is (+)-[8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine, or a pharmaceutically acceptable salt thereof.

22. A compound according to claim 20 which is an isomer thereof having a negative optical rotation that is (−)-[8-(3-fluoro-2-fluoromethyl-propyl)-6-methyl-5,6,7,8-tetrahydro-9-oxa-1,6-diaza-benzocyclohepten-2-yl]-[6-methoxy-5-(4-methyl-imidazol-1-yl)-pyridin-2-yl]-amine, or a pharmaceutically acceptable salt thereof.

23. A pharmaceutical composition comprising a compound or a pharmaceutically acceptable salt thereof according to claim 20 in association with a pharmaceutically acceptable excipient, carrier or diluent.

24. A pharmaceutical composition comprising a compound or a pharmaceutically acceptable salt thereof according to claim 21 in association with a pharmaceutically acceptable excipient, carrier or diluent.

25. A pharmaceutical composition according to claim 23, additionally comprising at least one cognitive enhancing agent, memory enhancing agent, acetylcholine esterase inhibitor, anti-inflammatory agent or atypical antipsychotic agent.

26. A pharmaceutical composition according to claim 24, additionally comprising at least one cognitive enhancing agent, memory enhancing agent, acetylcholine esterase inhibitor, anti-inflammatory agent or atypical antipsychotic agent.

27. A method of treating an Aβ-related pathology in a subject in need thereof wherein:
the Aβ-related pathology is selected from the group consisting of Down's syndrome, a β-amyloid angiopathy, cerebral amyloid angiopathy, hereditary cerebral hemorrhage, a disorder associated with cognitive impairment, mild cognitive impairment, Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy and cortical basal degeneration, and
the method comprises administering to the subject a therapeutically effective amount of a pharmaceutically composition according to claim 23.

28. A method of treating an Aβ-related pathology in a subject in need thereof wherein:
the Aβ-related pathology is selected from the group consisting of Down's syndrome, a β-amyloid angiopathy, cerebral amyloid angiopathy, hereditary cerebral hemorrhage, a disorder associated with cognitive impairment, mild cognitive impairment, Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy and cortical basal degeneration, and
the method comprises administering to the subject a therapeutically effective amount of a pharmaceutically composition according to claim 24.

29. A method of treating Alzheimer's disease comprising administering to a subject in need thereof a therapeutically effective amount of a pharmaceutical composition according to claim 23.

30. A method of treating Alzheimer's disease comprising administering to a subject in need thereof a therapeutically effective amount of a pharmaceutical composition according to claim 24.

31. A method of treating Alzheimer's disease comprising administering to a subject in need thereof a therapeutically effective amount of a pharmaceutical composition according to claim 23, and at least one cognitive enhancing agent, memory enhancing agent, acetylcholine esterase inhibitor, anti-inflammatory agent or atypical antipsychotic agent.

32. A method of treating Alzheimer's disease comprising administering to a subject in need thereof a therapeutically effective amount of a pharmaceutical composition according to claim 24, and at least one cognitive enhancing agent, memory enhancing agent, acetylcholine esterase inhibitor, anti-inflammatory agent or atypical antipsychotic agent.

33. A method of treating Alzheimer's disease comprising administering to subject in need thereof a therapeutically effective amount of a compound according to claim 20.

34. A method of treating Alzheimer's disease comprising administering to subject in need thereof a therapeutically effective amount of a compound according to claim 21.

35. A method of treating Alzheimer's disease comprising administering to a subject in need thereof a therapeutically effective amount of a compound according to claim 20, and at least one cognitive enhancing agent, memory enhancing agent, acetylcholine esterase inhibitor, anti-inflammatory agent or atypical antipsychotic agent.

36. A method of treating Alzheimer's disease comprising administering to a subject in need thereof a therapeutically effective amount of a compound according to claim 21, and at least one cognitive enhancing agent, memory enhancing agent, acetylcholine esterase inhibitor, anti-inflammatory agent or atypical antipsychotic agent.
